(19) United States
(12) Patent Application Publication
(10) Pub. No.: US 2026/0028588 A1
(43) Pub. Date: Jan. 29, 2026

(54) DIFFERENTIATION OF STEM CELLS IN SUSPENSION CULTURE

(71) Applicant: Umoja Biopharma, Inc., Seattle, WA (US)

(72) Inventors: Teisha J. ROWLAND, Seattle, WA (US); Cassidy FRIEDMAN, Seattle, WA (US); Ashley M. YINGST, Seattle, WA (US); Samantha O'HARA, Seattle, WA (US); Dillon JARRELL, Seattle, WA (US); David T. VEREIDE, Seattle, WA (US); Ryan KONING, Seattle, WA (US)

(73) Assignee: Umoja Biopharma, Inc., Seattle, WA (US)

(21) Appl. No.: 18/995,112

(22) PCT Filed: Jul. 27, 2023
(86) PCT No.: PCT/US2023/071097

§ 371 Date: Jan. 15, 2025

Related U.S. Application Data
(60) Provisional application No. 63/392,760, filed on Jul. 27, 2022.

(51) Int. Cl. C12N 5/0783; A61K 35/12; A61K 35/17; A61K 40/15; C12N 5/00; C12N 5/0789; C12N 5/10; C12N 9/22; C12N 15/113; C12N 15/63

(52) U.S. Cl. CPC C12N 5/0646; A61K 35/17; A61K 40/15; C12N 5/0062; C12N 5/0647; C12N 5/10; C12N 9/226; C12N 15/113; C12N 15/63; A61K 2035/124; C12N 2500/98; C12N 2500/99; C12N 2501/33; C12N 2506/02; C12N 2506/45; C12N 2510/00; C12N 2513/00

(57) ABSTRACT

Provided are xenogenic-free methods and compositions for generating hematopoietic progenitors and natural killer (NK) cells in 3D suspension culture.

### CROSS-REFERENCE TO RELATED APPLICATIONS

This application claims benefit of priority to U.S. Provisional Patent Application No. 63/392,760, filed Jul. 27, 2022, the disclosure of which is incorporated herein by reference in its entirety for all purposes.

### BACKGROUND

Natural Killer (NK) cells are a type of cytotoxic innate lymphoid cells generally identified as positive for the cell surface protein CD56 (CD56+) and other markers, and as having cytotoxic activity.

NK cells for use in immunotherapy can be obtained from primary sources such as peripheral blood or umbilical cord blood. Artificial sources for NK cells include pluripotent stem cells, including induced pluripotent stem cells (iPSCs), which are cells derived from somatic cells (generally fibroblasts or peripheral blood mononuclear cells (PBMCs), and human embryonic stem cells (hESCs), either induced to become capable of unlimited proliferation and of differentiation into other cell types when subjected to appropriate differentiation conditions. From iPSCs, NK cells may be derived by sequentially differentiating the iPSCs into hematopoietic progenitor cells (HPCs), also termed hematopoietic stem cells (HSCs).

Once obtained, primary NK or iPSC-NK cells can be expanded ex vivo before administration to patients. Methods for differentiating iPSCs into NK cells often involves the use of feeder cells or media with serum. To provide cells suitable for in vivo administration, there remains a need for compositions and methods related to generating NK cells without using xenogenic factors, such as animal-derived raw materials like fetal bovine serum (FBS) and/or feeder cells, and allowing for scale-up manufacturing.

### SUMMARY

The present disclosure is based, at least in part, on the discovery of a method for differentiating stem cells into hematopoietic progenitors and NK cells in suspension. As provided herein, hematopoietic progenitors and NK cells generated in a three-dimensional suspension culture system were of higher purity and exhibited higher frequency of desired cell markers relative to two-dimensional culture systems for differentiation. Without wishing to be bound by theory, the suspension culture methods described herein are suitable for large-scale manufacturing of hematopoietic progenitors and/or NK cells. Further, as demonstrated herein, the suspension culture methods are xenogenic free. Without wishing to be bound by theory, the xengoenic free method described herein results in cells suitable for in vivo administration.

In some embodiments, the disclosure provides a method for generating a population of CD34+/CD43+/CD45+ cells, comprising:
(i) culturing a population of progenitor cells in a two-dimensional (2D) culture system for a period of time sufficient to form progenitor cell aggregates;
(ii) passaging the progenitor cell aggregates from the 2D culture system to a three-dimensional (3D) suspension culture system;
(iii) contacting the progenitor cell aggregates in the 3D suspension culture system with a differentiation media for a period of time sufficient to generate the population of CD34+/CD43+/CD45+ cells.

In some embodiments, the disclosure provides a method for differentiating a population of stem cells into a population of hematopoietic progenitors, comprising:
(i) culturing the population of stem cells in a 2D culture system for a period of time sufficient to form stem cell aggregates;
(ii) passaging the stem cell aggregates from the 2D culture system to a 3D suspension culture system;
(iii) contacting the stem cell aggregates in the 3D suspension culture system with a differentiation media comprising a bone morphogenetic protein (BMP) pathway activator, a fibroblast growth factor (FGF), and a vascular endothelial growth factor (VEGF), for a period of time sufficient to differentiate the population of stem cells into the population of hematopoietic progenitors.

In some embodiments, the 3D suspension culture has a volume of between 50-50,000 ml.

In some embodiments, the 3D suspension culture is agitated. In some embodiments, the 3D suspension culture is agitated at a rate of between 10 revolutions per minute (RPM) to 100 RPM. In some embodiments, the 3D suspension culture is agitated at a rate of 70 RPM.

In some embodiments, the population of hematopoietic progenitors comprises CD34+/CD43+/CD45+ cells.

In some embodiments, the BMP pathway activator is BMP4. In some embodiments, the FGF is FGF2. In some embodiments, the VEGF is VEGF-165. In some embodiments, the differentiation media comprises Rho-associated coiled coil forming protein serine/threonine kinase (ROCK) inhibitor. In some embodiments, wherein the ROCK inhibitor is Y27632.

In some embodiments, the differentiation media comprises stem cell factor (SCF). In some embodiments, the differentiation media comprises thrombopoietin (TPO). In some embodiments, the differentiation media comprises a low-density lipoprotein (LDL).

In some embodiments, the differentiation media comprises the BMP pathway activator, the FGF, the VEGF, and the ROCK inhibitor. In some embodiments, the differentiation media comprises the BMP pathway activator, the FGF, the VEGF, SCF, TPO, and the LDL.

In some embodiments, (iii) comprises contacting the population of stem cell aggregates with the differentiation media for 1-5 days, wherein the differentiation media comprises the BMP pathway activator, the FGF, the VEGF, and optionally the ROCK inhibitor.

In some embodiments, (iii) comprises (a) contacting the stem cell aggregates for 1-5 days with the differentiation media comprising the BMP pathway activator, the FGF, the VEGF the ROCK inhibitor, to generate embryoid bodies or mesoderm cells, and (b) contacting the embryoid bodies or mesoderm cells for 1-15 days with a differentiation media comprising the BMP pathway activator, the FGF, the VEGF, SCF, TPO, and the LDL.

In some embodiments, the differentiation media comprises 1-50 ng/mL BMP, 1-50 ng/mL FGF, 5-100 ng/mL VEGF, 0.1-20 uM ROCK inhibitor, 1-200 ng/mL SCF, 1-100 ng/mL TPO, and 1-50 ug/mL LDL, or any combination thereof.

In some embodiments, the progenitor cells or stem cells are induced pluripotent stem cells (iPSCs). In some embodiments, the progenitor cells or stem cells are human embryonic stem cells (hESCs).

In some embodiments, the differentiation media is serum free. In some embodiments, the method is xenogenic-free.

In some embodiments, the disclosure provides a method of generating a population of NK cells, comprising:
(a) culturing a population of stem cells in a 2D culture system for a period of time sufficient to form stem cell aggregates;
(b) passaging the stem cell aggregates from the 2D culture system to a 3D suspension culture system;
(c) contacting the stem cell aggregates in the 3D suspension culture system with a first media comprising a BMP pathway activator, an FGF, a VEGF, and optionally an inhibitor of ROCK, for a period of time sufficient to generate embryoid bodies;
(d) contacting the embryoid bodies with a first differentiation media comprising a BMP pathway activator, a FGF, VEGF, SCF, TPO, and an LDL, for a period of time sufficient to generate a population of hematopoietic progenitors;
(e) contacting the population of hematopoietic progenitors with a second differentiation media, for a period of time sufficient to generate the population of NK cells.

In some embodiments, the second differentiation media comprising SCF, IL-7, IL-12, IL-15, FLT3L, a pyrimido-[4,5-b]-indole derivative, and an AhR inhibitor.

In some embodiments, the media comprises 1-100 ng/mL SCF, 1-50 ng/mL IL-7, 1-100 ng/mL IL-12, 1-100 ng/mL IL-15, 1-100 ng/mL FLT3L, 0.1-10 uM pyrimido-[4,5-b]-indole derivative, 0.1-10 uM AhR antagonist, and any combination thereof.

In some embodiments, the pyrimido-[4,5-b]-indole derivative is UM729 and the AhR inhibitor is SR1.

In some embodiments, the BMP pathway activator is BMP4, the FGF is FGF2, the VEGF is VEGF-165, and the inhibitor of ROCK is Y27632.

In some embodiments, each media of steps (b)-(e) is serum free. In some embodiments, the method is xenogenic-free.

In some embodiments, the first media, the first differentiation media, and the second differentiation media each comprise the same base media. In some embodiments, the first differentiation media, and the second differentiation media each comprise different base media. In some embodiments, the first differentiation media and the second differentiation media each comprise the same base media, and the first media comprises a base media different from the first and second differentiation media. In some embodiments, the first differentiation media and the second differentiation media each comprise a base media comprising Iscove's modified dulbecco's medium, bovine serum albumin, recombinant human insulin, human transferrin, and 2-mercaptoethanol.

In some embodiments, the period of time of step (b) is 2-8 days, the period of time of step (c) is 1-5 days, the period of time of step (d) is 3-15 days, and the period of time of step (e) is 11-25 days. In some embodiments, steps (a)-(e) occur within 40-50 days.

In some embodiments, the stem cells are induced pluripotent stem cells (iPSCs) or human embryonic stem cells (hESCs).

In some embodiments, the population of hematopoietic progenitors comprises about 50% to about 100% CD34+/CD43+/CD45+ cells. In some embodiments, the population of NK cells comprises about 60% to about 100% CD43+/CD45+/CD56+/LFA1+ cells. In some embodiments, the population of NK cells expands about 1,000 to about 10,000 fold.

In some embodiments, the population of stem cells is genetically engineered or edited. In some embodiments, the population of NK cells is genetically engineered or edited.

In some embodiments, the disclosure provides a population of cells comprising hematopoietic progenitors produced by a method described herein. In some embodiments, the hematopoietic progenitors are CD34+/CD43+/CD45+. In some embodiments, the cell population comprises 30-50% hematopoietic progenitors.

In some embodiments, the disclosure provides a population of cells comprising NK cells produced by a method described herein. In some embodiments, the NK cells are CD45+/CD56+/LFA1+. In some embodiments, the population of cells comprises 60-100% NK cells.

In some embodiments, the disclosure provides a pharmaceutical composition comprising the cell population of the disclosure.

In some embodiments, the disclosure provides a method of generating a population of hematopoietic progenitors, comprising:
(a) genetically engineering a population of stem cells to express a synthetic cytokine receptor for a non-physiological ligand,
   wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain;
(b) culturing the population of stem cells in a 2D culture system for a period of time sufficient to form stem cell aggregates;
(c) passaging the stem cell aggregates from the 2D culture system to a 3D suspension culture system; and
(d) contacting the stem cell aggregates in the 3D suspension culture system with a differentiation media for a period of time sufficient to generate hematopoietic progenitors.

In some embodiments, the disclosure provides a method of generating a population of NK cells, comprising:
(a) genetically engineering a population of stem cells to express a synthetic cytokine receptor for a non-physiological ligand,
   wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain;
(b) culturing the population of stem cells in a 2D culture system for a period of time sufficient to form stem cell aggregates;
(c) passaging the stem cell aggregates from the 2D culture system to a 3D suspension culture system;
(d) contacting the stem cell aggregates in the 3D suspension culture system with a first differentiation media for a period of time sufficient to generate hematopoietic progenitors; and
(e) contacting the population of hematopoietic progenitors with a second differentiation media, for a period of time sufficient to generate the population of NK cells.

In some embodiments, the intracellular domain of the synthetic beta chain polypeptide is selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

In some embodiments, the nucleotide sequence is inserted via homology directed repair (HDR).

In some embodiments, the vector comprises a nucleic acid comprising from 5′ to 3′ (a) a nucleotide sequence homologous with a region located upstream of the target site, (b) the nucleotide sequence encoding a synthetic cytokine receptor for a non-physiological ligand, and (c) a nucleotide sequence homologous with a region located downstream, wherein a double-strand break occurs at the target site in the endogenous gene, and the nucleic acid is exchanged with a homologous nucleotide sequence of the endogenous gene.

In some embodiments, the nucleotide sequence is inserted via non-homologous end joining (NHEJ).

In some embodiments, the cells are engineered with an RNA-guided endonuclease. In some embodiments, the RNA-guided endonuclease is selected from a Cas endonuclease, a Mad endonuclease, and a Cpf1 endonuclease. In some embodiments, the RNA-guided endonuclease is Cas9 or Mad7.

In some embodiments, the method comprises disrupting a target gene and inserting the nucleotide sequence into the disrupted target gene, wherein disrupting the target gene comprises contacting the population of stem cells with (i) a gRNA targeting a target site in a target gene, and (ii) an RNA-guided endonuclease. In some embodiments, the target gene is selected from B2M, TRAC and SIRPA.

In some embodiments, the population of stem cells is engineered to be resistant to rapamycin. In some embodiments, engineering the population of stem cells to be resistant to rapamycin comprises knocking out a FKBP12 gene.

In some embodiments, the differentiation media comprises a BMP pathway activator, an FGF, a VEGF, and optionally a ROCK inhibitor. In some embodiments, the BMP pathway activator is BMP4, the FGF is FGF2, the VEGF is VEGF-165, and the ROCK inhibitor is Y27632.

In some embodiments, the differentiation media comprises SCF, TPO and LDL.

In some embodiments, (d) comprises contacting the population of stem cell aggregates with the differentiation media for 1-5 days, wherein the differentiation media comprises a BMP pathway activator, an FGF, a VEGF, and optionally a ROCK inhibitor.

In some embodiments, (d) comprises (i) contacting the stem cell aggregates for 1-5 days with the differentiation media comprising a BMP pathway activator, an FGF, a VEGF, and a ROCK inhibitor, to generate embryoid bodies or mesoderm cells, and (ii) contacting the embryoid bodies or mesoderm cells for 1-15 days with a differentiation media comprising the BMP pathway activator, the FGF, the VEGF, SCF, TPO, and the LDL.

In some embodiments, the differentiation media comprises 1-50 ng/mL BMP, 1-50 ng/mL FGF, 5-100 ng/mL VEGF, 0.1-20 uM ROCK inhibitor, 1-200 ng/mL SCF, 1-100 ng/mL TPO, and 1-50 ug/mL LDL, or any combination thereof.

In some embodiments, the first differentiation media comprises a BMP pathway activator, an FGF, a VEGF, and optionally a ROCK inhibitor.

In some embodiments, (d) comprises contacting the population of stem cell aggregates with the first differentiation media for 1-5 days, wherein the first differentiation media comprises a BMP pathway activator, an FGF, a VEGF, and optionally a ROCK inhibitor.

In some embodiments, (d) comprises (i) contacting the stem cell aggregates for 1-5 days with a media comprising a BMP pathway activator, an FGF, a VEGF, and a ROCK inhibitor, to generate embryoid bodies or mesoderm cells, and (ii) contacting the embryoid bodies or mesoderm cells for 1-15 days with the first differentiation media comprising the BMP pathway activator, the FGF, the VEGF, SCF, TPO, and the LDL.

In some embodiments, the BMP pathway activator is BMP4, the FGF is FGF2, the VEGF is VEGF-165, and the ROCK inhibitor is Y27632.

In some embodiments, the second differentiation media comprises SCF, IL-7, IL-12, IL-15, FLT3L, a pyrimido-[4,5-b]-indole derivative, and an AhR inhibitor.

In some embodiments, the second differentiation media comprises 1-100 ng/mL SCF, 1-50 ng/mL IL-7, 1-100 ng/mL IL-12, 1-100 ng/mL IL-15, 1-100 ng/mL FLT3L, 0.1-10 uM pyrimido-[4,5-b]-indole derivative, 0.1-10 uM AhR antagonist, and any combination thereof.

In some embodiments, the pyrimido-[4,5-b]-indole derivative is UM729 and the AhR inhibitor is SR1.

In some embodiments, the first differentiation media and the second differentiation media are serum free.

In some embodiments, the method is xenogenic-free.

In some embodiments, the first differentiation media, and the second differentiation media each comprise the same base media. In some embodiments, the first differentiation media, and the second differentiation media each comprise different base media. In some embodiments, the first differentiation media and the second differentiation media each comprise a base media comprising Iscove's modified dulbecco's medium, bovine serum albumin, recombinant human insulin, human transferrin, and 2-mercaptoethanol.

In some embodiments, the period of time of step (b) is 2-8 days, the period of time of step (c) is 1-5 days, the period of time of step (d) is 3-15 days, and the period of time of step (e) is 11-25 days. In some embodiments, steps (a)-(e) occur within 40-50 days.

In some embodiments, the stem cells are induced pluripotent stem cells (iPSCs) or human embryonic stem cells (hESCs).

In some embodiments, the population of hematopoietic progenitors comprises about 50% to about 100% CD34+/CD43+/CD45+ cells.

In some embodiments, the population of NK cells comprises about 60% to about 100% CD43+/CD45+/CD56+/LFA1+ cells.

In some embodiments, the method comprises expanding the population of NK cells, wherein the population of NK cells expands about 1,000 to about 10,000 fold.

In some embodiments, the population of stem cells is genetically engineered or edited. In some embodiments, the population of NK cells is genetically engineered or edited.

In some embodiments, the stem cells are iPSCs.

In some embodiments, the disclosure provides a population of cells produced by the methods of the disclosure. In some embodiments, the disclosure provides a pharmaceutical composition comprising the cell population of the disclosure.

In some embodiments, comprising administering to the subject an effective amount of the population of cells or the pharmaceutical composition.

In some embodiments, the disclosure provides a kit comprising the population of cells and instructions for administering the cell population to a subject in need thereof.

In some embodiments, the subject has a cancer.

### BRIEF DESCRIPTION OF THE DRAWINGS

FIG. 1 provides a schematic showing an exemplary method (“method #1”) for differentiating stem cells into hematopoietic progenitors and NK cells in suspension culture.

FIG. 2 provides a schematic showing an exemplary method (“method #2”) for differentiating stem cells into hematopoietic progenitors in suspension culture.

FIGS. 3A-3E show characterization of hematopoietic progenitors (HPs) differentiated from stem cells based on the protocol provided in FIG. 1 and FIG. 2. HPs were characterized at day 15 unless indicated otherwise. FIG. 3A shows flow cytometry analysis performed by gating cells to quantify percentage of cells triple-positive for the HP markers CD34/CD43/CD45. FIG. 3B is a graph showing average HP purity, ranging from 60-80%, of all cells triple-positive for CD34/CD43/CD45. FIG. 3C is a graph showing the average for expansion of HPs relative to iPSCs seeded at day 0. FIG. 3D is a graph showing expansion of HPs generated using the indicated method at day 12 compared to a standard 2D differentiation protocol. FIG. 3E provides representative brightfield microscope images of the EBs prior to HP harvesting.

FIGS. 4A-4E show characterization of natural killer (NK) cells differentiated from stem cells using method 1. NK cells were characterized at day 40. FIG. 4A shows flow cytometry analysis performed by gating cells to quantify percentage of CD45+CD56+LFA1+ cells. FIG. 4B is a graph showing NK purity of all cells positive for CD34/CD45/CD56/LFA1. FIG. 4C is a graph showing expansion of NKs relative to iPSCs seeded at day 0. FIG. 4D is a graph showing expansion of NKs generated using method #1 compared to a standard 2D differentiation protocol. FIG. 4E provides representative brightfield microscope images of the NKs.

FIG. 5A and FIG. 5B show D40 differentiated iNKs incubated with breast adenocarcinoma MDA-MB231 cells at different T:E ratios. The NK cells and MDA-MB231 cells were incubated in the absence (unstimulated, FIG. 5A) or presence (stimulated, FIG. 5B) of cytokines IL-2 and IL-15. iNK cells reduced MDA growth in a dose-responsive manner.

FIG. 6 provides a schematic showing an exemplary experimental design for differentiating genetically engineered stem cells into hematopoietic progenitors and NK cells in suspension culture.

FIG. 7 is a graph showing the ratio of hematopoietic progenitor cells (HPs) to iPSCs of either parental wild-type cells or engineered FKBP12 knockout iPSCs that encode RACR (B2M-EF1a-RACR and FKBP12 KO cells) after 14 days in 3D suspension culture. Cells were transferred to 3D suspension culture using either gentle cell dissociation reagent (GCDR) or EDTA.

### DETAILED DESCRIPTION

In some aspects, the disclosure provides compositions and methods for generating hematopoietic progenitors, common lymphoid progenitors, pre-NK progenitors, NK progenitors, immature NK cells, and/or NK cells. In some embodiments, the methods described herein are in a three-dimensional culture system. In some embodiments, the compositions and methods described herein are xenogenic-free.

### Definitions

All publications, patent applications, patents and other references mentioned herein are incorporated by reference in their entirety.

Unless the context indicates otherwise, the various features described herein can be used in any combination with any feature or combination of features set forth herein, and each feature can be excluded or omitted from the combination.

As used herein, the singular forms “a”, “an”, and “the” are include the plural forms as well, unless the context indicates otherwise. The conjugation “and/or” denotes all possible combinations of one or more of listed items.

“Subject” as used herein refers to the recipient of an NK cell population generated by the methods of the disclosure. The term includes mammal, such as primate, mouse, rat, dog, cat, cow, horse, goat, camel, sheep or a pig, preferably a human.

“Treat,” “treating” or “treatment” as used herein refers to any type of action or administration that imparts a benefit to a subject that has a disease or disorder, including improvement in the condition of the patient (i.e., improvement, reduction, or amelioration of one or more symptoms, and partial or complete response to treatment).

The term “effective amount” refers to an amount effective to generate a desired biochemical, cellular, or physiological response. The term “therapeutically effective amount” refer to the amount, dosage, or dosage regime of a therapy effective to cause a desire treatment effect.

“Polynucleotide” as used herein refers to a biopolymer composed of two or more nucleotide monomers covalently bonded through ester linkages between the phosphoryl group of one nucleotide and the hydroxyl group of the sugar component of the next nucleotide in a chain. DNA and RNA are non-limiting examples of polynucleotides.

“Polypeptide” as used herein refers to a polymer consisting of amino acid residues chained together by peptide bonds, forming part of (or the whole of) a protein.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed.

Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The term “variant” means a polynucleotide or polypeptide having at least one substitution, insertion, or deletion in its sequence compared to a reference polynucleotide or polypeptide. A “functional variant” is a variant that retains one or functions of the reference polynucleotide or polypeptide.

The term “inactivating mutation” refers to a mutation in a genomic sequence that disrupts a function of a gene. The inactivating mutation can be in any sequence region (e.g., coding, or non-coding) that contributes to gene expression. Examples include, but are not limited to, cis-acting elements (enhancers) or sequences that are subject to transcription (e.g., mRNA transcript sequences). An inactivating mutation includes mutations that render a gene or its encoded protein non-functional or that reduce the function of the gene or its encoded protein.

As used herein the term “sequence identity”, or “identity” in relation to polynucleotides or polypeptide sequences, refers to the extent to which two optimally aligned polynucleotides or polypeptide sequences match at each position in the alignment across the full length of the reference sequence. The “percent identity” is the number of matched positions in the optimal alignment, divided by length of the reference sequence plus the sum of the lengths of any gaps in the reference sequence in the alignment. The optimal alignment is the alignment that results in the maximum percent identity. Alignment of sequences to determine percent identity can be accomplished by a number of well-known methods, including for example by using mathematical algorithms, such as, for example, those in the BLAST suite or Clustal Omega sequence analysis programs. Unless noted otherwise, the term “sequence identity” in the claims refers to sequence identity as calculated by BLAST version 2.12.0 using default parameters. And, unless noted otherwise, the alignment is an alignment of all or a portion of the polynucleotide or polypeptide sequences of interest across the full length of the reference sequence.

As used herein, the term “engineered” refers to a cell that has been stably transduced with a heterologous polynucleotide or subjected to gene editing to introduce, delete, or modify polynucleotides in the cell, or cells transiently transduced with a polynucleotide in a manner that causes a stable phenotypic change in the cell.

As used herein, the term “stem cell” is used to describe a cell with an undifferentiated phenotype, capable, for example, of differentiating into hematopoietic progenitors, and/or NK cells.

As used herein, the term “pluripotent” means the stem cell is capable of forming substantially all of the differentiated cell types of an organism, at least in culture. For example, embryonic stem cells are a type of pluripotent stem cells that are able to form cells from each of the three germs layers, the ectoderm, the mesoderm, and the endoderm.

As used herein, the terms “induced pluripotent stem cell” and “iPSC” are used to refer to cells, derived from somatic cells, that have been reprogrammed back to a pluripotent state and are capable of proliferation, selectable differentiation, and maturation. iPSCs are stem cells produced from differentiated adult, neonatal, or fetal cells that have been induced or changed, i.e., reprogrammed, into cells capable of differentiating into tissues of all three germ or dermal layers: mesoderm, endoderm, and ectoderm. The iPSCs produced do not refer to cells as they are found in nature.

As used herein, the terms “hematopoietic stem cell”, “hematopoietic progenitor”, or “hematopoietic progenitor cell” refer to stem cells capable of giving rise to both mature myeloid and lymphoid cell types including natural killer cells, T cells, and B cells. Hematopoietic stem cells are typically characterized as CD34+.

The term “progenitor” refers to a cell partially differentiated into a desired cell type. Progenitor cells retain a degree of pluripotency and may differentiate to multiple cell types.

As used herein, “differentiate” or “differentiated” are used to refer to the process and conditions by which undifferentiated, or immature (e.g., unspecialized), cells acquire characteristics becoming mature (specialized) cells thereby acquiring particular form and function. Stem cells (unspecialized) are often exposed to varying conditions (e.g., growth factors and morphogenic factors) to induce specified lineage commitment, or differentiation, of said stem cells.

As used herein, “expand” or “expansion” refer to an increase in the number and/or purity of a cell type within a cell population through mitotic division of cells having limited proliferative capacity, e.g., NK cells.

As used herein, “activity”, “activate”, or “activation” refer to stimulation of activating receptors on a cytotoxic innate lymphoid cell leading to cell division, cytokine secretion (e.g., IFNγ and/or TNFα), and/or release of cytolytic granules to regulate or assist in an immune response.

As used herein, “xenogenic free” refers to compositions and methods lacking animal-derived raw materials (e.g., fetal bovine serum). In some embodiments, the xengoenic free methods described herein do not include feeder cells.

As used herein, “2D culture” refers to growing cell cultures on a flat surface, such as the bottom of a petri dish or flask, wherein the cells are in contact with the flat surface.

As used herein, “3D suspension culture” refers to an artificially created environment in which cells are permitted to grow or interact with their surroundings in all three dimensions. 3D suspension culture allows cells in vitro to grow in all directions, similar to how they would in vivo. These three-dimensional cultures are usually grown in bioreactors, small capsules in which the cells can grow into spheroids, or 3D cell colonies.

The term “bioreactor” refers to any manufactured device or system that supports a biologically active environment. In some embodiments, a bioreactor is a vessel in which a process is carried out that allows organisms to grow.

### 3D Suspension Culture

In some embodiments, the disclosure provides a method for differentiating stem cells into hematopoietic progenitors in a 3D suspension culture. In some embodiments, the methods comprise passaging cells from 2D culture to 3D suspension culture. In some embodiments, the disclosure provides a method for differentiating hematopoietic progenitors into NK cells by culturing hematopoietic progenitors in a 3D suspension culture. In some embodiments, the disclosure provides media for expanding NK cells in suspension. In some embodiments, the differentiating and/or expansion media described herein comprise a serum-free base media with at least one exogenous factor to drive differentiation and/or expansion.

### 3D Suspension Culture Volume

In some embodiments, the cell populations of the disclosure are cultured in 3D suspension culture. In some embodiments, the volume of the 3D suspension culture is at least 0.01 ml, at least 0.1 ml, at least 1 ml, at least 10 ml, at least 100 ml, at least 1,000 ml, at least 10,000 ml, at least 100,000 ml, or at least 1,000,000 ml, including all intervening values between.

In some embodiments, the volume of the 3D suspension culture is about 0.01 mL to about 0.1 mL, about 0.1 mL to about 1 mL, about 1 mL to about 10 mL, about 10 mL to about 100 mL, about 100 mL to about 1,000 mL, about 1,000 mL to about 10,000 mL, about 10,000 mL to about 100,000 mL, or about 100,000 mL to about 1,000,000 mL, including all intervening values between. In some embodiments, the volume of the 3D suspension culture is about 1 mL to about 1,000,000 mL. In some embodiments, the volume of the 3D suspension culture is about 100 mL to about 1,000 mL. In some embodiments, the volume of the 3D suspension culture is about 200 mL to about 2,000 mL. In some embodiments, the volume of the 3D suspension culture is about 500 mL to about 2,000 mL. In some embodiments, the volume of the 3D suspension culture is about 1,000 mL to about 1,000,000 mL.

In some embodiments, 3D suspension culture volume is housed in a container. In some embodiments, the container is a flask, multi-layer flask, bottle, dish, or bioreactor.

In some embodiments, the bioreactor is a hollow fiber bioreactor, a packed bed bioreactor, a stirred tank bioreactor, a rocking motion bioreactor, a stirred tank bioreactor, and/or a wave bioreactor.

In some embodiments, the methods described herein are performed in a bioreactor. In some embodiments, the 3D culture suspension system is a bioreactor. In some embodiments, the 3D culture suspension system is a bioreactor of about 100 mL to about 1,000 mL. In some embodiments, the 3D culture suspension system is a bioreactor of about 200 mL to about 2,000 mL. In some embodiments, the 3D culture suspension system is a bioreactor of about 500 mL to about 2,000 mL.

### 3D Suspension Culture Agitation

In some embodiments, the 3D suspension culture is agitated. In some embodiments, the agitation is measured at a rate of revolutions per minute (RPM). In some embodiments, the 3D suspension culture is agitated at a rate of at least 1 RPM, at least 5 RPM, at least 10 RPM, at least 15 RPM, at least 20 RPM, at least 25 RPM, at least 30 RPM, at least 35 RPM, at least 40 RPM, at least 45 RPM, at least 50 RPM, at least 55 RPM, at least 60 RPM, at least 65 RPM, at least 70 RPM, at least 75 RPM, at least 80 RPM, at least 85 RPM, at least 90 RPM, at least 95 RPM, at least 100 RPM, at least 105 RPM, at least 110 RPM, at least 120 RPM, at least 130 RPM, at least 140 RPM, at least 150 RPM, at least 160 RPM, at least 170 RPM, at least 180 RPM, at least 190 RPM, at least 200 RPM, at least 250 RPM, at least 300 RPM, at least 350 RPM, at least 400 RPM, at least 450 RPM, at least 500 RPM, at least 550 RPM, at least 600 RPM, at least 650 RPM, at least 700 RPM, at least 750 RPM, at least 800 RPM, at least 850 RPM, at least 900 RPM, at least 950 RPM, or at least 1,000 RPM, including all intervening values between.

In some embodiments, the 3D suspension culture is agitated at a rate of about 1 RPM to about 5 RPM, about 5 RPM to about 10 RPM, about 10 RPM to about 15 RPM, about 15 RPM to about 20 RPM, about 20 RPM to about 25 RPM, about 25 RPM to about 30 RPM, about 30 RPM to about 35 RPM, about 35 RPM to about 40 RPM, about 40 RPM to about 45 RPM, about 45 RPM to about 50 RPM, about 50 RPM to about 55 RPM, about 55 RPM to about 60 RPM, about 60 RPM to about 65 RPM, about 65 RPM to about 70 RPM, about 70 RPM to about 75 RPM, about 75 RPM to about 80 RPM, about 80 RPM to about 85 RPM, about 85 RPM to about 90 RPM, about 90 RPM to about 95 RPM, about 95 RPM to about 100 RPM, about 100 RPM to about 105 RPM, about 105 RPM to about 115 RPM, about 110 RPM to about 120 RPM, about 120 RPM to about 130 RPM, about 130 RPM to about 140 RPM, about 140 RPM to about 150 RPM, about 150 RPM to about 160 RPM, about 160 RPM to about 170 RPM, about 170 RPM to about 180 RPM, about 180 RPM to about 190 RPM, about 190 RPM to about 200 RPM, about 200 RPM to about 250 RPM, about 250 RPM to about 300 RPM, about 300 RPM to about 350 RPM, about 350 RPM to about 400 RPM, about 400 RPM to about 450 RPM, about 450 RPM to about 500 RPM, about 500 RPM to about 550 RPM, about 550 RPM to about 600 RPM, about 600 RPM to about 650 RPM, about 650 RPM to about 700 RPM, about 700 RPM to about 750 RPM, about 750 RPM to about 800 RPM, about 800 RPM to about 850 RPM, about 850 RPM to about 900 RPM, about 900 RPM to about 950 RPM, or about 950 RPM to about 1,000 RPM, including all intervening values between.

In some embodiments, the 3D suspension culture is agitated at a rate of about 1 RPM to about 1,000 RPM. In some embodiments, the 3D suspension culture is agitated at a rate of about 10 RPM to about 500 RPM. In some embodiments, 3D suspension culture is agitated at a rate of about 50 RPM to about 100 RPM.

In some embodiments, the 3D suspension culture is agitated at a rate of about 70 RPM. In some embodiments, the 3D suspension culture is agitated at a rate of 70 RPM.

### 3D Suspension Culture Media Change

In some embodiments, the media of the 3D suspension culture is changed at least once during the methods described herein. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% of the media volume of the 3D suspension culture is changed, including all intervening values between.

In some embodiments, about 1% to 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 70% to 75%, about 75% to 80/c, about 80% to 85%, about 85% to 90%, about 90% to 95%, or about 95% to 100% of the media volume of the 3D suspension culture is changed, including all intervening values between.

In some embodiments, the media change occurs on day 1, on day 2, on day 3, on day 4, on day 5, on day 6, on day 7, on day 8, on day 9, on day 10, on day 11, on day 12, on day 13, on day 14, on day 15, on day 16, on day 17, on day 18, on day 19, on day 20, on day 21, on day 22, on day 23, on day 24, on day 25, on day 26, on day 27, on day 28, on day 29, on day 30, on day 31, on day 32, on day 33, on day 34, on day 35, on day 36, on day 37, on day 38, on day 39, on day 40, on day 41, on day 42, on day 43, on day 44, on day 45, on day 46, on day 47, on day 48, on day 49, or on day 50 of differentiation.

In some embodiments, the media is changed at least once a week and not on the other days. For example, in some embodiments, the media is changed on day 1 and not on days 2 to 7 of the week. In some embodiments, the media is changed on day 2 and not on days 1 and 3 to 7 of the week. In some embodiments, the media is changed on day 3 and not on days 1 to 2 and 4 to 7 of the week. In some embodiments, the media is changed on day 4 and not on days 1 to 3 and 5 to 7 of the week. In some embodiments, the media is changed on day 5 and not on days 1 to 4 and 6 to 7 of the week. In some embodiments, the media is changed on day 6 and not on days 1 to 5 and 7 of the week. In some embodiments, the media is changed on day 7 and not on days 1 to 6 of the week.

In some embodiments, the media is changed at least twice a week. In some embodiments, the media is changed on days 1 and 2 and not on days 3 to 7 of the week. In some embodiments, the media is changed on days 1 and 3 and not on days 2 and 4 to 7 of the week. In some embodiments, the media is changed on days 1 and 4 and not on days 2 to 3 and 5 to 7 of the week. In some embodiments, the media is changed on days 1 and 5 and not on days 2 to 4 and 6 to 7 of the week. In some embodiments, the media is changed on days 1 and 6 and not on days 2 to 5 and 6 to 7 of the week. In some embodiments, the media is changed on days 1 and 7 and not on days 2 to 6 of the week. In some embodiments, the media is changed on days 2 and 3 and not on days 2 and 3 to 7 of the week. In some embodiments, the media is changed on days 2 and 4 and not on days 1, 3 and 5 to 7 of the week. In some embodiments, the media is changed on days 2 and 5 and not on days 1, 3, 5, 6 and 7 of the week. In some embodiments, the media is changed on days 2 and 6 and not on days 1, 3, 4, 5, and 7 of the week. In some embodiments, the media is changed on days 2 and 7 and not on days 1 and 3 to 6 of the week. In some embodiments, the media is changed on days 3 and 4 and not on days 1, 2 and 5 to 7 of the week. In some embodiments, the media is changed on days 3 and 5 and not on days 1, 2, 4 and 6 to 7 of the week. In some embodiments, the media is changed on days 3 and 6 and not on days 1, 2, 4, 5 and 7 of the week. In some embodiments, the media is changed on days 3 and 7 and not on days 1, 2, 4, 5 and 7 of the week. In some embodiments, the media is changed on days 4 and 5 and not on days 1 to 3 and 6 to 7 of the week. In some embodiments, the media is changed on days 4 and 6 and not on days 1 to 3, 5 and 7 of the week. In some embodiments, the media is changed on days 4 and 7 and not on days 1 to 3, 5, and 6 of the week. In some embodiments, the media is changed on days 5 and 6 and not on days 1 to 4 and 7 of the week. In some embodiments, the media is changed on days 5 and 7 and not on days 1 to 4 and 6 of the week. In some embodiments, the media is changed on days 6 and 7 and not on days 1 to 5 of the week.

In some embodiments, the media is changed at least three times a week. In some embodiments, the media is changed on days 1 to 3 and is not on days 4 to 7. In some embodiments, the media is changed on days 1, 2, and 4 and is not on days 3 and 5 to 7 of the week. In some embodiments, the media is changed on days 1, 2, and 5 and is not on days 3, 4, 6, and 7 of the week. In some embodiments, the media is changed on days 1, 2, and 6 and is not on days 3, 4, 5 and 7 of the week. In some embodiments, the media is changed on days 1, 2, and 7 and is not on days 3 to 6 of the week. In some embodiments, the media is changed on days 1, 3 and 4 and is not on days 2, and 5 to 7. In some embodiments, the media is changed on days 1, 3, and 5 and is not on days 4, 5, 6 and 7 of the week. In some embodiments, the media is changed on days 1, 3, and 6 and is not on days 2 to 5 and 7 of the week. In some embodiments, the media is changed on days 1, 3, and 7 and is not on days 2 and 4 to 6 of the week. In some embodiments, the media is changed on days 1, 3, and 7 and is not on days 2 and 4 to 6 of the week. In some embodiments, the media is changed on days 1, 4 and 5 and is not on days 2, 3, 6 and 7. In some embodiments, the media is changed on days 1, 4, and 6 and is not on days 2 to 4 and 7 of the week. In some embodiments, the media is changed on days 1, 4, and 7 and is not on days 2, 3, 5 and 6 of the week. In some embodiments, the media is changed on days 1, 5, and 6 and is not on days 2 to 4 and 7 of the week. In some embodiments, the media is changed on days 1, 5, and 7 and is not on days 2 and 4 to 6 of the week. In some embodiments, the media is changed on days 2, 3 and 4 and is not on days 1 and 5 to 7. In some embodiments, the media is changed on days 2, 3, and 5 and is not on days 1, 4, 6 and 7 of the week. In some embodiments, the media is changed on days 2, 3, and 6 and is not on days 1, 4, 5 and 7 of the week. In some embodiments, the media is changed on days 2, 3, and 7 and is not on days 1, 4 to 6 of the week. In some embodiments, the media is changed on days 2, 4 and 5 and is not on days 1, 3, 6 and 7. In some embodiments, the media is changed on days 2, 4, and 6 and is not on days 1, 3, 5 and 7 of the week. In some embodiments, the media is changed on days 2, 4, and 7 and is not on days 1, 3, 5 and 6 of the week. In some embodiments, the media is changed on days 2, 5, and 6 and is not on days 1, 3, 4 and 7 of the week. In some embodiments, the media is changed on days 2, 5, and 7 and is not on days 1, 3, 4, and 6 of the week. In some embodiments, the media is changed on days 2, 6, and 7 and is not on days 1, 3, 4 and 5 of the week. In some embodiments, the media is changed on days 3, 4, and 5 and is not on days 1, 2, 6 and 7 of the week. In some embodiments, the media is changed on days 3, 4, and 6 and is not on days 1, 2, 5 and 7 of the week. In some embodiments, the media is changed on days 3, 4, and 7 and is not on days 1, 2, 5 and 6 of the week. In some embodiments, the media is changed on days 3, 5, and 6 and is not on days 1, 2, 4 and 7 of the week. In some embodiments, the media is changed on days 3, 5, and 7 and is not on days 1, 2, 4 and 6 of the week. In some embodiments, the media is changed on days 3, 6, and 7 and is not on days 1, 2, 4 and 5 of the week. In some embodiments, the media is changed on days 4, 5, and 6 and is not on days 1 to 3 and 7 of the week. In some embodiments, the media is changed on days 4, 5, and 7 and is not on days 1 to 3 and 6 of the week. In some embodiments, the media is changed on days 4, 6, and 7 and is not on days 1 to 3 and 5 of the week. In some embodiments, the media is changed on days 5, 6, and 7 and is not on days 1 to 4 of the week.

In some embodiments, the media is changed at least four times a week. In some embodiments, the media is not changed on days 1 to 3 and is changed on days 4 to 7. In some embodiments, the media is not changed on days 1, 2, and 4 and is changed on days 3 and 5 to 7 of the week. In some embodiments, the media is not changed on days 1, 2, and 5 and is changed on days 3, 4, 6, and 7 of the week. In some embodiments, the media is not changed on days 1, 2, and 6 and is changed on days 3, 4, 5 and 7 of the week. In some embodiments, the media is not changed on days 1, 2, and 7 and is changed on days 3 to 6 of the week. In some embodiments, the media is not changed on days 1, 3 and 4 and is changed on days 2, and 5 to 7. In some embodiments, the media is not changed on days 1, 3, and 5 and is changed on days 4, 5, 6 and 7 of the week. In some embodiments, the media is not changed on days 1, 3, and 6 and is changed on days 2 to 5 and 7 of the week. In some embodiments, the media is not changed on days 1, 3, and 7 and is changed on days 2 and 4 to 6 of the week. In some embodiments, the media is not changed on days 1, 3, and 7 and is changed on days 2 and 4 to 6 of the week. In some embodiments, the media is not changed on days 1, 4 and 5 and is changed on days 2, 3, 6 and 7. In some embodiments, the media is not changed on days 1, 4, and 6 and is changed on days 2 to 4 and 7 of the week. In some embodiments, the media is not changed on days 1, 4, and 7 and is changed on days 2, 3, 5 and 6 of the week. In some embodiments, the media is not changed on days 1, 5, and 6 and is changed on days 2 to 4 and 7 of the week. In some embodiments, the media is not changed on days 1, 5, and 7 and is changed on days 2 and 4 to 6 of the week. In some embodiments, the media is not changed on days 2, 3 and 4 and is changed on days 1 and 5 to 7. In some embodiments, the media is not changed on days 2, 3, and 5 and is changed on days 1, 4, 6 and 7 of the week. In some embodiments, the media is not changed on days 2, 3, and 6 and is changed on days 1, 4, 5 and 7 of the week. In some embodiments, the media is not changed on days 2, 3, and 7 and is changed on days 1, 4 to 6 of the week. In some embodiments, the media is not changed on days 2, 4 and 5 and is changed on days 1, 3, 6 and 7. In some embodiments, the media is not changed on days 2, 4, and 6 and is changed on days 1, 3, 5 and 7 of the week. In some embodiments, the media is not changed on days 2, 4, and 7 and is changed on days 1, 3, 5 and 6 of the week. In some embodiments, the media is not changed on days 2, 5, and 6 and is changed on days 1, 3, 4 and 7 of the week. In some embodiments, the media is not changed on days 2, 5, and 7 and is changed on days 1, 3, 4, and 6 of the week. In some embodiments, the media is not changed on days 2, 6, and 7 and is changed on days 1, 3, 4 and 5 of the week. In some embodiments, the media is not changed on days 3, 4, and 5 and is changed on days 1, 2, 6 and 7 of the week. In some embodiments, the media is not changed on days 3, 4, and 6 and is changed on days 1, 2, 5 and 7 of the week. In some embodiments, the media is not changed on days 3, 4, and 7 and is changed on days 1, 2, 5 and 6 of the week. In some embodiments, the media is not changed on days 3, 5, and 6 and is changed on days 1, 2, 4 and 7 of the week. In some embodiments, the media is not changed on days 3, 5, and 7 and is changed on days 1, 2, 4 and 6 of the week. In some embodiments, the media is not changed on days 3, 6, and 7 and is changed on days 1, 2, 4 and 5 of the week. In some embodiments, the media is not changed on days 4, 5, and 6 and is changed on days 1 to 3 and 7 of the week. In some embodiments, the media is not changed on days 4, 5, and 7 and is changed on days 1 to 3 and 6 of the week. In some embodiments, the media is not changed on days 4, 6, and 7 and is changed on days 1 to 3 and 5 of the week. In some embodiments, the media is not changed on days 5, 6, and 7 and is changed on days 1 to 4 of the week.

In some embodiments, the media is changed at least five times a week. In some embodiments, the media is not changed on days 1 and 2 and is changed on days 3 to 7 of the week. In some embodiments, the media is not changed on days 1 and 3 and is changed on days 2 and 4 to 7 of the week. In some embodiments, the media is not changed on days 1 and 4 and is changed on days 2 to 3 and 5 to 7 of the week. In some embodiments, the media is not changed on days 1 and 5 and is changed on days 2 to 4 and 6 to 7 of the week. In some embodiments, the media is not changed on days 1 and 6 and is changed on days 2 to 5 and 6 to 7 of the week. In some embodiments, the media is not changed on days 1 and 7 and is changed on days 2 to 6 of the week. In some embodiments, the media is not changed on days 2 and 3 and is changed on days 2 and 3 to 7 of the week. In some embodiments, the media is not changed on days 2 and 4 and is changed on days 1, 3 and 5 to 7 of the week. In some embodiments, the media is not changed on days 2 and 5 and is changed on days 1, 3, 5, 6 and 7 of the week. In some embodiments, the media is not changed on days 2 and 6 and is changed on days 1, 3, 4, 5, and 7 of the week. In some embodiments, the media is not changed on days 2 and 7 and is changed on days 1 and 3 to 6 of the week. In some embodiments, the media is not changed on days 3 and 4 and is changed on days 1, 2 and 5 to 7 of the week. In some embodiments, the media is not changed on days 3 and 5 and is changed on days 1, 2, 4 and 6 to 7 of the week. In some embodiments, the media is not changed on days 3 and 6 and is changed on days 1, 2, 4, 5 and 7 of the week. In some embodiments, the media is not changed on days 3 and 7 and is changed on days 1, 2, 4, 5 and 7 of the week. In some embodiments, the media is not changed on days 4 and 5 and is changed on days 1 to 3 and 6 to 7 of the week. In some embodiments, the media is not changed on days 4 and 6 and is changed on days 1 to 3, 5 and 7 of the week. In some embodiments, the media is not changed on days 4 and 7 and is changed on days 1 to 3, 5, and 6 of the week. In some embodiments, the media is not changed on days 5 and 6 and is changed on days 1 to 4 and 7 of the week. In some embodiments, the media is not changed on days 5 and 7 and is changed on days 1 to 4 and 6 of the week. In some embodiments, the media is not changed on days 6 and 7 and is changed on days 1 to 5 of the week.

In some embodiments, the media is changed at least six times a week and is changed on the other days. For example, the media is not changed on day 1 and is changed on days 2 to 7 of the week. In some embodiments, the media is not changed on day 1 and is changed on days 2 to 7 of the week. In some embodiments, the media is not changed on day 2 and is changed on days 1 and 3 to 7 of the week. In some embodiments, the media is not changed on day 3 and is changed on days 1 to 2 and 4 to 7 of the week. In some embodiments, the media is not changed on day 4 and is changed on days 1 to 3 and 5 to 7 of the week. In some embodiments, the media is not changed on day 5 and is changed on days 1 to 4 and 6 to 7 of the week. In some embodiments, the media is not changed on day 6 and is changed on days 1 to 5 and 7 of the week. In some embodiments, the media is not changed on day 7 and is changed on days 1 to 6 of the week.

In some embodiments, the media is changed each day of the week.

### 3D Suspension Culture Seeding Density

In some embodiments, the cell populations of the disclosure are seeded in the 3D suspension culture at a density of at least 1×10³ cells, at least 1×10⁴ cells, at least 1×10⁵ cells, at least 1×10⁶ cells, at least 1×10⁷ cells, at least 1×10⁸ cells, or at least 1×10⁹ cells.

In some embodiments, the cell populations of the disclosure are seeded in the 3D suspension culture at a density of about 1×10³ cells to 1×10⁴ cells, about 1×10⁴ cells to 1×10⁵ cells, about 1×10⁵ cells 1×10⁶ cells, about 1×10⁶ cells to 1×10⁷ cells, about 1×10⁷ cells to 1×10⁸ cells, or about 1×10⁸ cells 1×10⁹ cells.

### Differentiation and Expansion Media

In some embodiments, the disclosure provides media for differentiating stem cells into hematopoietic progenitors. In some embodiments, the disclosure provides media for differentiating hematopoietic progenitors into NK cells. In some embodiments, the disclosure provides media for expanding NK cells. In some embodiments, the differentiating and/or expansion media described herein comprise a serum-free base media with at least one exogenous factor to drive differentiation and/or expansion.

### Xenogenic-Free Media

In some embodiments, in the differentiation and/or expansion media described herein is a defined media. As used herein, “defined media” refers to a growth medium suitable for the in vitro culture of human or animal cells in which all of the chemical components are known. In some embodiments, the differentiation and/or expansion media comprises a base media. In some embodiments, the base media comprises Iscove's Modified Dulbecco's Medium, serum albumin, human insulin, human transferrin, and 2-mercaptoethanol. In some embodiments, the base media comprises human serum albumin. In some embodiments, the base media does not include animal-derived raw materials.

In some embodiments, the base media is selected from StemSpan SFEM II Medium (STEMCELL Technologies; serum-free), Stemline II (Sigma-Aldrich; fully defined, serum- and animal component-free, GMP manufactured), CTS NK Xpander Medium (Gibco; serum-free and animal component-free medium), STEMdiff Hematopoietic—EB Basal Medium (STEMCELL Technologies; serum-free), Stemdiff APEL 2 medium (STEM CELL Technologies; serum-free and animal component-free) or Hematopoietic Progenitor Expansion Medium XF (PromoCell: serum-free and xeno-free medium). In some embodiments, the base media is StemSpan SFEM II media. In some embodiments, the base media is Stemline II media. In some embodiments, the base media is Stemdiff APEL 2 media. In some embodiments, the hematopoietic progenitor differentiation media and the NK cell differentiation media have the same base media. In some embodiments, the hematopoietic progenitor differentiation media and the NK cell differentiation media have different base media.

### Erogenous Factors

In some embodiments, the differentiation and/or expansion media described herein comprises exogenous factors. In some embodiments, the methods of the disclosure comprise contacting different cell populations with various exogenous factors in xenogenic-free media to drive differentiation of cells to e.g., hematopoietic progenitors and/or NK cells. In some embodiments, the exogenous factors include but are not limited to cytokines e.g., interleukins, fibroblast growth factors (FGF), stem cell factor (SCF), Phosphatidylinositol 3-kinases (PI3K) inhibitors, FMS-like tyrosine kinase 3 ligand (FLT3L), Bone morphogenetic protein (BMP) pathway activators, pyrimido-indole derivatives, and aryl hydrocarbon receptor antagonists.

In some embodiments, an exogenous factor suitable for use in a differentiation and/or expansion media is a cytokine. Cytokines include interferons, interleukins and growth factors, which are small proteins that play an important role in cell signaling. In some embodiments, the cytokine is an interleukin. In some embodiments, the interleukin is selected from IL-2, IL-7, IL-12, IL-15, IL-18, and any combination thereof. In some embodiments, the cytokine is a growth factor. In some embodiments, the growth factor is selected from a fibroblast growth factor, a vascular endothelial growth factor, and any combination thereof.

In some embodiments, the exogenous factor is interleukin 2 (IL-2). IL-2 is a secreted cytokine produced by activated CD4+ and CD8+ T lymphocytes, that is important for the proliferation of T and B lymphocytes. IL-2 is a member of the interleukin 2 (IL2) cytokine subfamily which includes IL-4, IL-7, IL-9, IL-15, IL-21, erythropoietin, and thrombopoietin.

In some embodiments, the exogenous factor is interleukin 7 (IL-7). IL-7 is a member of the interleukin 2 (IL2) cytokine subfamily. Lymphoid differentiation and activation critically depend on IL-7 signaling.

In some embodiments, the exogenous factor is interleukin 12 (IL-12). IL-12 a cytokine that acts on T and natural killer cells, and has a broad array of biological activities. NK cells may acquire memory-like properties following a brief stimulation with IL-12.

In some embodiments, the exogenous factor is interleukin 15 (IL-15). IL-15 is a member of the interleukin 2 (IL2) cytokine subfamily. IL-15 regulates NK cell activation and proliferation.

In some embodiments, the exogenous factor is interleukin 18 (IL-18). IL-18 is a proinflammatory cytokine of the IL-1 family that is constitutively found as a precursor within the cytoplasm of a variety of immune cells. IL-18 has been shown to potently activate NK cells.

In some embodiments, the exogenous factor is Low-density lipoprotein (LDL). LDL induces an increase in proliferation and cytotoxic activity of NK cells.

In some embodiments, the exogenous factor is a fibroblast growth factor (FGF). The FGF family members are cell signaling proteins produced by macrophages. The FGF family comprises 23 members. In some embodiments, the exogenous factor is FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23. In some embodiments, the exogenous factor is FGF2.

In some embodiments, the exogenous factor is FMS-like tyrosine kinase 3 ligand (FLT3L). FLT3L is an essential growth factor for NK cells and has been shown to play an important role in the expansion of early hematopoietic progenitors and in the generation of mature peripheral NK cells.

In some embodiments, the exogenous factor is stem cell factor (SCF). SCF plays an important role in the survival of stem cells and the self-renewal and maintenance of stem cells.

In some embodiments, the exogenous factor is a vascular endothelial growth factor (VEGF). The VEGF family is a sub-family of growth factors, the platelet-derived growth factor family of cystine-knot growth factors. The VEGF family comprises five family members. In some embodiments, the exogenous factor is VEGF-A, placenta growth factor (PGF), VEGF-B, VEGF-C and VEGF-D. In some embodiments, the exogenous factor is VEGF-165. VEGF165 is a 38.2 kDa, disulfide-linked homodimeric protein consisting of two 165 amino acid polypeptide chains.

In some embodiments, the exogenous factor is an aryl hydrocarbon inhibitor. The aryl hydrocarbon receptor is a transcription factor that regulates gene expression. The aryl hydrocarbon receptor has roles in regulating immunity, stem cell maintenance, and cellular differentiation. Antagonism of the aryl hydrocarbon receptor has been shown to promote the renewal and expansion of stem cells. In some embodiments, the exogenous factor is an antagonists of the aryl hydrocarbon receptor selected from PD98059, StemRegenin 1 (SR1), GNF351, BAY 2416964, CH-223191, Perillaldehyde, PDM-11, and BAY-218. In some embodiments, the exogenous factor is SR1.

In some embodiments, the exogenous factor is an inhibitor of phosphatidylinositol 3-kinases (PI3Ks). PI3Ks comprise a family of lipid and serine/threonine kinases that catalyze the transfer of phosphate to the D-3′ position of inositol lipids to produce phosphoinositol-3-phosphate (PIP), phosphoinositol-3,4-diphosphate (PIP2) and phosphoinositol-3,4,5-triphosphate (PIP3) that, in turn, act as second messengers in signaling cascades by docking proteins containing pleckstrin-homology, FYVE, Phox and other phospholipid-binding domains into a variety of signaling complexes often at the plasma membrane.

Inhibitors of PI3Ks include, but are not limited to, Idelalisib, Copanlisib, Duvelisib, Alpelisib, Umbralisib, Buparlisib, Copanlisib, Dactolisib, Duvelisib, Idelalisib, Leniolisib, Parsaclisib, Paxalisib, Taselisib, Zandelisib, Inavolisib, Apitolisib, Bimiralisib, Eganelisib, Fimepinostat, Gedatolisib, Linperlisib, Nemiralisib, Pictilisib, Pilaralisib, Samotolisib, Seletalisib, Serabelisib, Sonolisib, Tenalisib, Voxtalisib, AMG 319, AZD8186, GSK2636771, SF1126, Acalisib, Omipalisib, AZD8835, CAL263, GSK1059615, MEN1611, PWT33597, TG100-115, ZSTK474, AEZS-136, B591, GNE-477, Hibiscone C, IC87114, LY294002, and PI-103. In some embodiments, the exogenous factor is Idelalisib, Copanlisib, Duvelisib, Alpelisib, Umbralisib, Buparlisib, Copanlisib, Dactolisib, Duvelisib, Idelalisib, Leniolisib, Parsaclisib, Paxalisib, Taselisib, Zandelisib, Inavolisib, Apitolisib, Bimiralisib, Eganelisib, Fimepinostat, Gedatolisib, Linperlisib, Nemiralisib, Pictilisib, Pilaralisib, Samotolisib, Seletalisib, Serabelisib, Sonolisib, Tenalisib, Voxtalisib, AMG 319, AZD8186, GSK2636771, SF1126, Acalisib, Omipalisib, AZD8835, CAL263, GSK1059615, MEN1611, PWT33597, TG100-115, ZSTK474, AEZS-136, B591, GNE-477, Hibiscone C, IC87114, LY294002, PI-103, or any combination thereof. In some embodiments, the exogenous factor is LY294002.

In some embodiments, the exogenous factor is an activator of the BMP pathway. Bone morphogenetic proteins (BMPs) are produced as large precursor molecules which are processed proteolytically to mature peptides after translation. BMPs act through specific transmembrane receptors located on cell surface of the target cells. The BMP receptors are serin-threonin kinases which resemble TGF-β receptors and are divided into two subgroups: type I and type II receptors. BMPs can bind strongly only to the heterotetrameric complex of these receptors. This complex formation is essential to the BMP signal transduction. Inside the target cell, BMP signals are transmitted to the nucleus via specific signal molecules called Smads, which are also responsible for suppression of BMP signals.

BMPs are multifunctional cytokines which are members of the transforming growth factor-beta superfamily. BMP receptors mediate BMP signaling through activating Smad. BMP ligands bind to the BMP receptors BMPRI and BMPRII. Phosphorylated BMPRII activates BMPRI. Phosphorylated BMPRI subsequently phosphorylates receptor-activated Smad proteins (R-Smads), which associate with common mediator-Smad (co-Smad) and enter the nucleus, where they regulate gene expression. BMP pathway activators include those agents disclosed in WO 2014011540, WO 2014062138, and WO 2005117994, which are incorporated herein by reference. BMP pathway activators include, but are not limited to, BMP-5, BMP-6, BMP-7, BMP-8, BMP-2, and BMP-4. In some embodiments, the BMP pathway activator is BMP-4. In some embodiments, the exogenous factor is BMP-4.

In some embodiments, the exogenous factor is an inhibitor of ROCK. Rho associated kinases (ROCK) are serine/threonine kinases that serve downstream effectors of Rho kinases (of which three isoforms exist—RhoA, RhoB and RhoC). ROCK inhibitors include, but are not limited to, polynucleotides, polypeptides, and small molecules. ROCK inhibitors contemplated herein may decrease ROCK expression and/or ROCK activity. Illustrative examples of ROCK inhibitors contemplated herein include, but are not limited to, anti-ROCK antibodies, dominant negative ROCK variants, siRNA, shRNA, miRNA and antisense nucleic acids that target ROCK.

Illustrative ROCK inhibitors contemplated herein include, but are not limited to: thiazovivin, Y27632, Fasudil, AR122-86, Y27632 H-1152, Y-30141, Wf-536, HA-1077, hydroxyl-HA-1077, GSK269962A, SB-772077-B, N-(4-Pyridyl)-N′-(2,4,6-trichlorophenyl)urea, 3-(4-Pyridyl)-1H-indole, and (R)-(+)-trans-N-(4-Pyridyl)-4-(1-aminoethyl)-cyclohexanecarboxamide, and ROCK inhibitors disclosed in U.S. Pat. No. 8,044,201, which is herein incorporated by reference in its entirety. In some embodiments, the ROCK inhibitor is thiazovivin, Y27632, or pyrintegrin. In some embodiments, the ROCK inhibitor is Y27632.

### Mesoderm/Embryoid Body Differentiation Media

In some embodiments, the disclosure provides a differentiation media for generating mesoderm and/or embryoid bodies from stem cells. In some embodiments, mesoderm cells are generated from iPSCs or hESCs. As stem cells begin to differentiate, three distinct germ layers are formed: the ectoderm, mesoderm, and endoderm. Immune cells, such as NK cells, differentiate from mesoderm cells. Embryoid bodies are three-dimensional aggregates that can differentiate into cells of all three germ layers. In some embodiments, the mesoderm cells are produced from embryoid bodies. In some embodiments, the mesoderm cells produced by the compositions and methods of the disclosure are further differentiated to hematopoietic progenitors. In some embodiments, the mesoderm cells produced by the compositions and methods of the disclosure are further differentiated into NK cells.

In some embodiments, a population of stem cells (e.g., iPSCs or hESCs) is cultured with at least one exogenous factor to form mesoderm and/or embryoid body cells. In some embodiments, the exogenous factor is a bone morphogenetic protein (BMP) activator. In some embodiments, the exogenous factor is an FGF. In some embodiments, the exogenous factor is a VEGF. In some embodiments, the exogenous factor is a ROCK inhibitor. In some embodiments, the exogenous factors are selected from a BMP pathway activator, a FGF, a VEGF, a ROCK inhibitor, and any combination thereof. In some embodiments, the exogenous factors comprise a BMP pathway activator and a FGF. In some embodiments, the exogenous factors comprise a BMP pathway activator and a VEGF. In some embodiments, the exogenous factors comprise a BMP pathway activator and a ROCK inhibitor. In some embodiments, the exogenous factors comprise a FGF and a VEGF. In some embodiments, the exogenous factors comprise a FGF and a ROCK inhibitor. In some embodiments, the exogenous factors comprise a VEGF and a ROCK inhibitor. In some embodiments, the exogenous factors comprise a BMP pathway activator, a FGF and a VEGF. In some embodiments, the exogenous factors comprise a BMP pathway activator, a FGF, and a ROCK inhibitor. In some embodiments, the exogenous factors comprise a FGF, a VEGF and a ROCK inhibitor. In some embodiments, the exogenous factors comprise a BMP pathway activator, a FGF, and a ROCK inhibitor. In some embodiments, the exogenous factors comprise a BMP pathway activator, a FGF, a VEGF, and a ROCK inhibitor.

In some embodiments, the exogenous factors comprise BMP4 and FGF2. In some embodiments, the exogenous factors comprise BMP4 and VEGF-165. In some embodiments, the exogenous factors comprise BMP4 and Y27632. In some embodiments, the exogenous factors comprise FGF2 and VEGF-165. In some embodiments, the exogenous factors comprise FGF2 and Y27632. In some embodiments, the exogenous factors comprise VEGF-165 and Y27632. In some embodiments, the exogenous factors comprise BMP4, FGF2 and VEGF-165. In some embodiments, the exogenous factors comprise BMP4, FGF2 and Y27632. In some embodiments, the exogenous factors comprise FGFs, VEGF-165 and Y27632. In some embodiments, the exogenous factors comprise BMP4, FGF2, and Y27632. In some embodiments, the exogenous factors comprise BMP4, FGF2, VEGF-165 and Y27632

In some embodiments, BMP4, FGF2, VEGF, and/or ROCK inhibitor are used in the mesoderm formation step. For example, the mesoderm formation step may include contacting the cell population with BMP4 and FGF2; with BMP4, FGF2 and a ROCK inhibitor; with BMP4 and VEGF; with BMP4, VEGF and a ROCK inhibitor; with FGF2 and VEGF; with FGF2, VEGF, with a ROCK inhibitor; BMP4, FGF2, and VEGF; BMP4, FGF2, VEGF, and a ROCK inhibitor; or individually any one of BMP4, FGF2, VEGF, and a ROCK inhibitor without the others.

In some embodiments, the bone morphogenetic protein (BMP) activator is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, the bone morphogenetic protein (BMP) activator is present in differentiation media at about 1-50 ng/ml.

In some embodiments, the BMP pathway activator is BMP4. In some embodiments, BMP4 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, BMP4 is present in differentiation media at about 1-50 ng/ml.

In some embodiments, FGF2 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 110 ng/ml, about 120 ng/ml, about 130 ng/ml, about 140 ng/ml, about 150 ng/ml, about 160 ng/ml, about 170, ng/ml, about 180 ng/ml, about 190 ng/ml, about 200 ng/ml, about 250 ng/ml, about 300 ng/ml, about 350 ng/ml, about 400 ng/ml, about 450 ng/ml, or about 500 ng/ml, or any range derivable therein. In some embodiments, FGF2 is present in differentiation media at about 1-100 ng/ml.

In some embodiments, VEGF is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, VEGF is present in differentiation media at about 5-100 ng/ml.

In some embodiments, the ROCK inhibitor is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 jM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, the ROCK inhibitor is present in differentiation media at about 0.1-20 μM.

In some embodiments, the ROCK inhibitor is Y27632. In some embodiments, Y27632 is present at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, about or 100 μM, or any range derivable therein. In some embodiments, Y27632 is present in differentiation media at about 0.1-100 μM.

In some embodiments, the mesoderm differentiation media comprises a BMP pathway activator, a FGF and a VEGF. In some embodiments, the mesoderm differentiation media comprises a BMP pathway activator, a FGF, a VEGF and a ROCK inhibitor. In some embodiments, the mesoderm differentiation media comprises a defined xenogenic-free base media, a BMP pathway activator, a FGF and a VEGF. In some embodiments, the mesoderm differentiation media comprises a defined xenogenic-free base media, a BMP pathway activator, a FGF, a VEGF, and a ROCK inhibitor. In some embodiments, the mesoderm differentiation media comprise BMP4, FGF2 and VEGF-165. In some embodiments, the mesoderm differentiation media comprises BMP4, FGF, VEGF-165 and a ROCK inhibitor. In some embodiments, the mesoderm differentiation media comprises BMP4, FGF, VEGF-165 and Y27632.

In some embodiments, the mesoderm differentiation media comprise 1-50 ng/mL BMP4, 1-150 ng/mL FGF2 and 1-100 ng/mL VEGF-165. In some embodiments, the mesoderm differentiation media comprises 1-50 ng/mL BMP4, 1-50 ng/mL FGF, 1-100 ng/mL VEGF-165 and 0.1-20 μM of a ROCK inhibitor. In some embodiments, the mesoderm differentiation media comprises 1-50 ng/mL BMP4, 1-50 ng/mL FGF, 1-100 ng/mL VEGF-165 and 0.1-20 μM Y27632.

### Hematopoietic Progenitor Differentiation Media

In some embodiments, the disclosure provides a differentiation media for generating HP cells from mesoderm cells and embryoid body cells. In some embodiments, the mesoderm cells and embryoid body cells produced by the compositions and methods of the disclosure are further differentiated to hematopoietic progenitors.

In some embodiments, a population of HP cells are cultured with at least one exogenous factor to form differentiated NK cells. In some embodiments, the exogenous factor is a BMP pathway activator. In some embodiments, the exogenous factor is exogenous factor is an FGF. In some embodiments, the exogenous factor a VEGF. In some embodiments, the exogenous factor is SCF. In some embodiments, the exogenous factor is TPO. In some embodiments, the exogenous factor is LDL. In some embodiments, the exogenous factor is a PI3K inhibitor. In some embodiments, the exogenous factor is a pyrimido-indole derivative. In some embodiments, the exogenous factor is an aryl hydrocarbon receptor antagonist. In some embodiments, the exogenous factor is a TGF-β receptor inhibitor. In some embodiments, the exogenous factors are selected from a BMP pathway activator, an FGF, a VEGF, SCF, TPO, LDL, a PI3K inhibitor, and any combination thereof. In some embodiments, the exogenous factors are selected from a BMP pathway activator, an FGF, a VEGF, SCF, TPO, LDL, and any combination thereof. In some embodiments, the exogenous factors are selected from a BMP pathway activator, an FGF, a VEGF, SCF, TPO, LDL, a PI3K inhibitor, a pyrimido-indole derivative, a aryl hydrocarbon receptor antagonist, a TGF-β receptor inhibitor, and any combination thereof.

In some embodiments, the exogenous factors comprise a BMP pathway activator and an FGF. In some embodiments, the exogenous factors comprise a BMP pathway activator and a VEGF. In some embodiments, the exogenous factors comprise a BMP pathway activator and SCF. In some embodiments, the exogenous factors comprise a BMP pathway activator and TPO. In some embodiments, the exogenous factors comprise a BMP pathway activator and LDL. In some embodiments, the exogenous factors comprise a BMP pathway activator and a PI3K inhibitor. In some embodiments, the exogenous factors comprise an FGF and a VEGF. In some embodiments, the exogenous factors comprise an FGF and SCF. In some embodiments, the exogenous factors comprise an FGF and TPO. In some embodiments, the exogenous factors comprise an FGF and LDL. In some embodiments, the exogenous factors comprise an FGF and a PI3K inhibitor. In some embodiments, the exogenous factors comprise a VEGF and SCF. In some embodiments, the exogenous factors comprise a VEGF and TPO. In some embodiments, the exogenous factors comprise a VEGF and LDL. In some embodiments, the exogenous factors comprise a VEGF and a PI3K inhibitor. In some embodiments, the exogenous factors comprise SCF and TPO. In some embodiments, the exogenous factors comprise SCF and LDL. In some embodiments, the exogenous factors comprise SCF and a PI3K inhibitor. In some embodiments, the exogenous factors comprise TPO and LDL. In some embodiments, the exogenous factors comprise TPO and a PI3K inhibitor. In some embodiments, the exogenous factors comprise LDL and a PI3K inhibitor.

In some embodiments, the exogenous factors comprise a BMP pathway activator, an FGF, and a VEGF. In some embodiments, the exogenous factors comprise a BMP pathway activator, an FGF, and SCF. In some embodiments, the exogenous factors comprise a BMP pathway activator, an FGF, and TPO. In some embodiments, the exogenous factors comprise a BMP pathway activator, an FGF, and LDL. In some embodiments, the exogenous factors comprise a BMP pathway activator, an FGF, and a PI3K inhibitor. In some embodiments, the exogenous factors comprise a BMP pathway activator, a VEGF, and SCF. In some embodiments, the exogenous factors comprise a BMP pathway activator, a VEGF, and TPO. In some embodiments, the exogenous factors comprise a BMP pathway activator, a VEGF, and LDL. In some embodiments, the exogenous factors comprise a BMP pathway activator, a VEGF, and a PI3K inhibitor. In some embodiments, the exogenous factors comprise a BMP pathway activator, SCF, and TPO. In some embodiments, the exogenous factors comprise a BMP pathway activator, SCF, and LDL. In some embodiments, the exogenous factors comprise a BMP pathway activator, SCF, and a PI3K inhibitor. In some embodiments, the exogenous factors comprise a BMP pathway activator, TPO, and LDL. In some embodiments, the exogenous factors comprise a BMP pathway activator, TPO, and a PI3K inhibitor. In some embodiments, the exogenous factors comprise a BMP pathway activator, LDL, and a PI3K inhibitor. In some embodiments, the exogenous factors comprise an FGF, a VEGF, and SCF. In some embodiments, the exogenous factors comprise an FGF, a VEGF, and TPO. In some embodiments, the exogenous factors comprise an FGF, a VEGF, and LDL. In some embodiments, the exogenous factors comprise an FGF, a VEGF, and LDL. In some embodiments, the exogenous factors comprise an FGF, SCF, and TPO. In some embodiments, the exogenous factors comprise an FGF, SCF, and LDL. In some embodiments, the exogenous factors comprise an FGF, SCF, and a PI3K inhibitor. In some embodiments, the exogenous factors comprise an FGF, TPO, and LDL. In some embodiments, the exogenous factors comprise an FGF, TPO, and a PI3K inhibitor. In some embodiments, the exogenous factors comprise an FGF, LDL, and a PI3K inhibitor. In some embodiments, the exogenous factors comprise a VEGF, SCF, and TPO. In some embodiments, the exogenous factors comprise a VEGF, SCF, and LDL. In some embodiments, the exogenous factors comprise a VEGF, SCF, and a PI3K inhibitor. In some embodiments, the exogenous factors comprise a VEGF, TPO, and LDL. In some embodiments, the exogenous factors comprise a VEGF, TPO, and a PI3K inhibitor.

In some embodiments, the exogenous factors comprise a BMP pathway activator, an FGF, a VEGF, and SCF. In some embodiments, the exogenous factors comprise a BMP pathway activator, an FGF, a VEGF, and TPO. In some embodiments, the exogenous factors comprise a BMP pathway activator, an FGF, a VEGF, and LDL. In some embodiments, the exogenous factors comprise a BMP pathway activator, an FGF, a VEGF, and a PI3K inhibitor. In some embodiments, the exogenous factors comprise a BMP pathway activator, an FGF, SCF, and TPO. In some embodiments, the exogenous factors comprise a BMP pathway activator, an FGF, SCF, and LDL. In some embodiments, the exogenous factors comprise a BMP pathway activator, an FGF, SCF, and a PI3K inhibitor. In some embodiments, the exogenous factors comprise a BMP pathway activator, an FGF, TPO, and LDL. In some embodiments, the exogenous factors comprise a BMP pathway activator, an FGF, TPO, and a PI3K inhibitor. In some embodiments, the exogenous factors comprise a BMP pathway activator, an FGF, LDL, and a PI3K inhibitor. In some embodiments, the exogenous factors comprise a BMP pathway activator, a VEGF, SCF, and TPO. In some embodiments, the exogenous factors comprise a BMP pathway activator, a VEGF, SCF, and LDL. In some embodiments, the exogenous factors comprise a BMP pathway activator, a VEGF, SCF, and a PI3K inhibitor. In some embodiments, the exogenous factors comprise a BMP pathway activator, a VEGF, TPO, and LDL. In some embodiments, the exogenous factors comprise a BMP pathway activator, a VEGF, TPO, and a PI3K inhibitor. In some embodiments, the exogenous factors comprise a BMP pathway activator, a VEGF, LDL, and a PI3K inhibitor. In some embodiments, the exogenous factors comprise a BMP pathway activator, SCF, TPO, and LDL. In some embodiments, the exogenous factors comprise a BMP pathway activator, SCF, TPO, and a PI3K inhibitor. In some embodiments, the exogenous factors comprise a BMP pathway activator, SCF, TPO, and a PI3K inhibitor. In some embodiments, the exogenous factors comprise a BMP pathway activator, TPO, LDL, and a PI3K inhibitor. In some embodiments, the exogenous factors comprise an FGF, a VEGF, SCF, and TPO. In some embodiments, the exogenous factors comprise an FGF, a VEGF, SCF, and LDL. In some embodiments, the exogenous factors comprise an FGF, a VEGF, SCF, and a PI3K inhibitor. In some embodiments, the exogenous factors comprise an FGF, a VEGF, TPO, and LDL. In some embodiments, the exogenous factors comprise an FGF, a VEGF, TPO, and a PI3K inhibitor. In some embodiments, the exogenous factors comprise an FGF, a VEGF, LDL, and a PI3K inhibitor. In some embodiments, the exogenous factors comprise an FGF, SCF, TPO, and LDL. In some embodiments, the exogenous factors comprise an FGF, SCF, TPO, and a PI3K inhibitor. In some embodiments, the exogenous factors comprise an FGF, SCF, LDL, and a PI3K inhibitor. In some embodiments, the exogenous factors comprise an FGF, TPO, LDL, and a PI3K inhibitor.

In some embodiments, the exogenous factors comprise a BMP pathway activator, an FGF, a VEGF, SCF, and TPO. In some embodiments, the exogenous factors comprise a BMP pathway activator, an FGF, a VEGF, SCF, and LDL. In some embodiments, the exogenous factors comprise a BMP pathway activator, an FGF, a VEGF, SCF, and a PI3K inhibitor. In some embodiments, the exogenous factors comprise a BMP pathway activator, an FGF, a VEGF, TPO, and LDL. In some embodiments, the exogenous factors comprise a BMP pathway activator, an FGF, a VEGF, TPO, and a PI3K inhibitor. In some embodiments, the exogenous factors comprise a BMP pathway activator, an FGF, a VEGF, LDL, and a PI3K inhibitor. In some embodiments, the exogenous factors comprise a BMP pathway activator, an FGF, SCF, TPO, and LDL. In some embodiments, the exogenous factors comprise a BMP pathway activator, an FGF, SCF, TPO, and a PI3K inhibitor. In some embodiments, the exogenous factors comprise a BMP pathway activator, an FGF, SCF, LDL, and a PI3K inhibitor. In some embodiments, the exogenous factors comprise a BMP pathway activator, an FGF, TPO, LDL, and a PI3K inhibitor. In some embodiments, the exogenous factors comprise a BMP pathway activator, a VEGF, SCF, TPO, and LDL. In some embodiments, the exogenous factors comprise a BMP pathway activator, a VEGF, SCF, TPO, and a PI3K inhibitor. In some embodiments, the exogenous factors comprise a BMP pathway activator, a VEGF, SCF, LDL, and a PI3K inhibitor. In some embodiments, the exogenous factors comprise a BMP pathway activator, a VEGF, TPO, LDL, and a PI3K inhibitor. In some embodiments, the exogenous factors comprise a BMP pathway activator, SCF, TPO, LDL, and a PI3K inhibitor. In some embodiments, the exogenous factors comprise an FGF, a VEGF, SCF, TPO, and LDL. In some embodiments, the exogenous factors comprise an FGF, a VEGF, SCF, TPO, and a PI3K inhibitor. In some embodiments, the exogenous factors comprise an FGF, a VEGF, SCF, LDL, and a PI3K inhibitor. In some embodiments, the exogenous factors comprise an FGF, a VEGF, TPO, LDL, and a PI3K inhibitor. In some embodiments, the exogenous factors comprise an FGF, a VEGF, SCF, TPO, and LDL. In some embodiments, the exogenous factors comprise an FGF, a VEGF, SCF, TPO, and a PI3K inhibitor. In some embodiments, the exogenous factors comprise an FGF, a VEGF, SCF, LDL, and a PI3K inhibitor. In some embodiments, the exogenous factors comprise an FGF, a VEGF, TPO, LDL, and a PI3K inhibitor. In some embodiments, the exogenous factors comprise an FGF, SCF, TPO, LDL, and a PI3K inhibitor. In some embodiments, the exogenous factors comprise a VEGF, SCF, TPO, LDL, and a PI3K inhibitor.

In some embodiments, the exogenous factors comprise a BMP pathway activator, an FGF, a VEGF, SCF, TPO, and LDL. In some embodiments, the exogenous factors comprise a BMP pathway activator, an FGF, a VEGF, SCF, TPO, and a PI3K inhibitor. In some embodiments, the exogenous factors comprise a BMP pathway activator, an FGF, a VEGF, SCF, LDL, and a PI3K inhibitor. In some embodiments, the exogenous factors comprise a BMP pathway activator, an FGF, a VEGF, TPO, LDL, and a PI3K inhibitor. In some embodiments, the exogenous factors comprise a BMP pathway activator, an FGF, SCF, TPO, LDL, and a PI3K inhibitor. In some embodiments, the exogenous factors comprise a BMP pathway activator, a VEGF, SCF, TPO, LDL, and a PI3K inhibitor. In some embodiments, the exogenous factors comprise an FGF, a VEGF, SCF, TPO, LDL, and a PI3K inhibitor.

In some embodiments, the exogenous factors comprise an BMP pathway activator, FGF, a VEGF, SCF, TPO, LDL, and a PI3K inhibitor.

In some embodiments, the exogenous factors comprise BMP4 and FGF2. In some embodiments, the exogenous factors comprise BMP4 and VEGF-165. In some embodiments, the exogenous factors comprise BMP4 and SCF. In some embodiments, the exogenous factors comprise BMP4 and TPO. In some embodiments, the exogenous factors comprise BMP4 and LDL. In some embodiments, the exogenous factors comprise BMP4 and LY294002. In some embodiments, the exogenous factors comprise FGF2 and VEGF-165. In some embodiments, the exogenous factors comprise FGF2 and SCF. In some embodiments, the exogenous factors comprise FGF2 and TPO. In some embodiments, the exogenous factors comprise FGF2 and LDL. In some embodiments, the exogenous factors comprise FGF2 and LY294002. In some embodiments, the exogenous factors comprise VEGF-165 and SCF. In some embodiments, the exogenous factors comprise VEGF-165 and TPO. In some embodiments, the exogenous factors comprise VEGF-165 and LDL. In some embodiments, the exogenous factors comprise VEGF-165 and LY294002. In some embodiments, the exogenous factors comprise SCF and TPO. In some embodiments, the exogenous factors comprise SCF and LDL. In some embodiments, the exogenous factors comprise SCF and LY294002. In some embodiments, the exogenous factors comprise TPO and LDL. In some embodiments, the exogenous factors comprise TPO and LY294002. In some embodiments, the exogenous factors comprise LDL and LY294002.

In some embodiments, the exogenous factors comprise BMP4, FGF2, and VEGF-165. In some embodiments, the exogenous factors comprise BMP4, FGF2, and SCF. In some embodiments, the exogenous factors comprise BMP4, FGF2, and TPO. In some embodiments, the exogenous factors comprise BMP4, FGF2, and LDL. In some embodiments, the exogenous factors comprise BMP4, FGF2, and LY294002. In some embodiments, the exogenous factors comprise BMP4, VEGF-165, and SCF. In some embodiments, the exogenous factors comprise BMP4, VEGF-165, and TPO. In some embodiments, the exogenous factors comprise BMP4, VEGF-165, and LDL. In some embodiments, the exogenous factors comprise BMP4, VEGF-165, and LY294002. In some embodiments, the exogenous factors comprise BMP4, SCF, and TPO. In some embodiments, the exogenous factors comprise BMP4, SCF, and LDL. In some embodiments, the exogenous factors comprise BMP4, SCF, and LY294002. In some embodiments, the exogenous factors comprise BMP4, TPO, and LDL. In some embodiments, the exogenous factors comprise BMP4, TPO, and LY294002. In some embodiments, the exogenous factors comprise BMP4, LDL, and LY294002. In some embodiments, the exogenous factors comprise FGF2, VEGF-165, and SCF. In some embodiments, the exogenous factors comprise FGF2, VEGF-165, and TPO. In some embodiments, the exogenous factors comprise FGF2, VEGF-165, and LDL. In some embodiments, the exogenous factors comprise FGF2, VEGF-165, and LDL. In some embodiments, the exogenous factors comprise FGF2, SCF, and TPO. In some embodiments, the exogenous factors comprise FGF2, SCF, and LDL. In some embodiments, the exogenous factors comprise FGF2, SCF, and LY294002. In some embodiments, the exogenous factors comprise FGF2, TPO, and LDL. In some embodiments, the exogenous factors comprise FGF2, TPO, and LY294002. In some embodiments, the exogenous factors comprise FGF2, LDL, and LY294002. In some embodiments, the exogenous factors comprise VEGF-165, SCF, and TPO. In some embodiments, the exogenous factors comprise VEGF-165, SCF, and LDL. In some embodiments, the exogenous factors comprise VEGF-165, SCF, and LY294002. In some embodiments, the exogenous factors comprise VEGF-165, TPO, and LDL. In some embodiments, the exogenous factors comprise VEGF-165, TPO, and LY294002.

In some embodiments, the exogenous factors comprise BMP4, FGF2, VEGF-165, and SCF. In some embodiments, the exogenous factors comprise BMP4, FGF2, VEGF-165, and TPO. In some embodiments, the exogenous factors comprise BMP4, FGF2, VEGF-165, and LDL. In some embodiments, the exogenous factors comprise BMP4, FGF2, VEGF-165, and LY294002. In some embodiments, the exogenous factors comprise BMP4, FGF2, SCF, and TPO. In some embodiments, the exogenous factors comprise BMP4, FGF2, SCF, and LDL. In some embodiments, the exogenous factors comprise BMP4, FGF2, SCF, and LY294002. In some embodiments, the exogenous factors comprise BMP4, FGF2, TPO, and LDL. In some embodiments, the exogenous factors comprise BMP4, FGF2, TPO, and LY294002. In some embodiments, the exogenous factors comprise BMP4, FGF2, LDL, and LY294002. In some embodiments, the exogenous factors comprise BMP4, VEGF-165, SCF, and TPO. In some embodiments, the exogenous factors comprise BMP4, VEGF-165, SCF, and LDL. In some embodiments, the exogenous factors comprise BMP4, VEGF-165, SCF, and LY294002. In some embodiments, the exogenous factors comprise BMP4, VEGF-165, TPO, and LDL. In some embodiments, the exogenous factors comprise BMP4, VEGF-165, TPO, and LY294002. In some embodiments, the exogenous factors comprise BMP4, VEGF-165, LDL, and LY294002. In some embodiments, the exogenous factors comprise BMP4, SCF, TPO, and LDL. In some embodiments, the exogenous factors comprise BMP4, SCF, TPO, and LY294002. In some embodiments, the exogenous factors comprise BMP4, SCF, TPO, and LY294002. In some embodiments, the exogenous factors comprise BMP4, TPO, LDL, and LY294002. In some embodiments, the exogenous factors comprise FGF2, VEGF-165, SCF, and TPO. In some embodiments, the exogenous factors comprise FGF2, VEGF-165, SCF, and LDL. In some embodiments, the exogenous factors comprise FGF2, VEGF-165, SCF, and LY294002. In some embodiments, the exogenous factors comprise FGF2, VEGF-165, TPO, and LDL. In some embodiments, the exogenous factors comprise FGF2, VEGF-165, TPO, and LY294002. In some embodiments, the exogenous factors comprise FGF2, VEGF-165, LDL, and LY294002. In some embodiments, the exogenous factors comprise FGF2, SCF, TPO, and LDL. In some embodiments, the exogenous factors comprise FGF2, SCF, TPO, and LY294002. In some embodiments, the exogenous factors comprise FGF2, SCF, LDL, and LY294002. In some embodiments, the exogenous factors comprise FGF2, TPO, LDL, and LY294002.

In some embodiments, the exogenous factors comprise BMP4, FGF2, VEGF-165, SCF, and TPO. In some embodiments, the exogenous factors comprise BMP4, FGF2, VEGF-165, SCF, and LDL. In some embodiments, the exogenous factors comprise BMP4, FGF2, VEGF-165, SCF, and LY294002. In some embodiments, the exogenous factors comprise BMP4, FGF2, VEGF-165, TPO, and LDL. In some embodiments, the exogenous factors comprise BMP4, FGF2, VEGF-165, TPO, and LY294002. In some embodiments, the exogenous factors comprise BMP4, FGF2, VEGF-165, LDL, and LY294002. In some embodiments, the exogenous factors comprise BMP4, FGF2, SCF, TPO, and LDL. In some embodiments, the exogenous factors comprise BMP4, FGF2, SCF, TPO, and LY294002. In some embodiments, the exogenous factors comprise BMP4, FGF2, SCF, LDL, and LY294002. In some embodiments, the exogenous factors comprise BMP4, FGF2, TPO, LDL, and LY294002. In some embodiments, the exogenous factors comprise BMP4, VEGF-165, SCF, TPO, and LDL. In some embodiments, the exogenous factors comprise BMP4, VEGF-165, SCF, TPO, and LY294002. In some embodiments, the exogenous factors comprise BMP4, VEGF-165, SCF, LDL, and LY294002. In some embodiments, the exogenous factors comprise BMP4, VEGF-165, TPO, LDL, and LY294002. In some embodiments, the exogenous factors comprise BMP4, SCF, TPO, LDL, and LY294002. In some embodiments, the exogenous factors comprise FGF2, VEGF-165, SCF, TPO, and LDL. In some embodiments, the exogenous factors comprise FGF2, VEGF-165, SCF, TPO, and LY294002. In some embodiments, the exogenous factors comprise FGF2, VEGF-165, SCF, LDL, and LY294002. In some embodiments, the exogenous factors comprise FGF2, VEGF-165, TPO, LDL, and LY294002. In some embodiments, the exogenous factors comprise FGF2, VEGF-165, SCF, TPO, and LDL. In some embodiments, the exogenous factors comprise FGF2, VEGF-165, SCF, TPO, and LY294002. In some embodiments, the exogenous factors comprise FGF2, VEGF-165, SCF, LDL, and LY294002. In some embodiments, the exogenous factors comprise FGF2, VEGF-165, TPO, LDL, and LY294002. In some embodiments, the exogenous factors comprise FGF2, SCF, TPO, LDL, and LY294002. In some embodiments, the exogenous factors comprise VEGF-165, SCF, TPO, LDL, and LY294002.

In some embodiments, the exogenous factors comprise BMP4, FGF2, VEGF-165, SCF, TPO, and LDL. In some embodiments, the exogenous factors comprise BMP4, FGF2, VEGF-165, SCF, TPO, and LY294002. In some embodiments, the exogenous factors comprise BMP4, FGF2, VEGF-165, SCF, LDL, and LY294002. In some embodiments, the exogenous factors comprise BMP4, FGF2, VEGF-165, TPO, LDL, and LY294002. In some embodiments, the exogenous factors comprise BMP4, FGF2, SCF, TPO, LDL, and LY294002. In some embodiments, the exogenous factors comprise BMP4, VEGF-165, SCF, TPO, LDL, and LY294002. In some embodiments, the exogenous factors comprise FGF2, VEGF-165, SCF, TPO, LDL, and LY294002.

In some embodiments, the exogenous factors comprise BMP4, FGF2, VEGF-165, SCF, TPO, LDL, and LY294002.

In some embodiments, the bone morphogenetic protein (BMP) activator is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, the bone morphogenetic protein (BMP) activator is present in differentiation media at about 1-50 ng/ml.

In some embodiments, the bone morphogenetic protein (BMP) activator is BMP4. In some embodiments, BMP4 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, BMP4 is present in differentiation media at about 1-50 ng/ml.

In some embodiments, FGF2 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, FGF2 is present in differentiation media at about 1-50 ng/ml.

In some embodiments, VEGF is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, VEGF is present in differentiation media at about 1-100 ng/ml.

In some embodiments, SCF is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, SCF is present in differentiation media at about 1-100 ng/ml.

In some embodiments, TPO is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, TPO is present in differentiation media at about 1-100 ng/ml.

In some embodiments, LDL is present in the differentiation media at a concentration of about 0.1-500 μg/ml, about 1-250 μg/ml, about 1-150 μg/ml, about 5-100 μg/ml, about or about 0.1 μg/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, about 10 μg/ml, about 11 μg/ml, about 12 μg/ml, about 13 μg/ml, about 14 μg/ml, about 15 sg/ml, about 16 μg/ml, about 17 μg/ml, about 18 μg/ml, about 19 μg/ml, about 20 μg/ml, about 21 μg/ml, about 22 μg/ml, about 23 μg/ml, about 24 μg/ml, about 25 μg/ml, about 26 μg/ml, about 27 μg/ml, about 28 μg/ml, about 29 μg/ml, about 30 μg/ml, about 35 μg/ml, about 40 μg/ml, about 45 μg/ml, about 50 μg/ml, about 55 μg/ml, about 60 μg/ml, about 65 μg/ml, about 70 μg/ml, about 75 μg/ml, about 80 μg/ml, about 85 μg/ml, about 90 μg/ml, about 95 μg/ml, about or 100 μg/ml, or any range derivable therein. In some embodiments, LDL is present in differentiation media at about 1-50 μg/ml.

In some embodiments, the PI3K inhibitor is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, the PI3K inhibitor is present in differentiation media at about 0.1-100 μM.

In some embodiments, the PI3K inhibitor is LY294002. In some embodiments, LY294002 is present at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, about or 100 μM, or any range derivable therein. In some embodiments, LY294002 is present in differentiation media at about 0.1-100 μM.

In some embodiments, the pyrimido-indole derivative is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, the pyrimido-indole derivative is present in differentiation media at about 0.1-10 μM.

In some embodiments, the pyrimido-indole derivative is UM729. In some embodiments, UM729 is present at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, about or 100 μM, or any range derivable therein. In some embodiments, UM729 is present in differentiation media at about 0.1-10 μM.

In some embodiments, the aryl hydrocarbon receptor antagonist is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, the aryl hydrocarbon receptor antagonist is present in differentiation media at about 0.1-10 μM.

In some embodiments, the aryl hydrocarbon receptor antagonist is StemRegenin 1 (SR1). In some embodiments, SR1 is present at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, about or 100 μM, or any range derivable therein. In some embodiments, SR1 is present in differentiation media at about 0.1-10 μM.

In some embodiments, a TGF-β receptor inhibitor is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, the TGF-β receptor inhibitor is present in differentiation media at about 0.1-20 μM.

In some embodiments, the TGF-β receptor inhibitor is GW788388. In some embodiments, GW788388 is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, GW788388 is present in differentiation media at about 0.1-20 μM.

In some embodiments, the TGF-β receptor inhibitor is SB431542. In some embodiments, GW788388 is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, SB431542 is present in differentiation media at about 0.1-20 μM.

In some embodiments, the HP differentiation media comprises a BMP pathway activator, a FGF and a VEGF. In some embodiments, the HP differentiation media comprises a BMP pathway activator, a FGF, a VEGF and a ROCK inhibitor. In some embodiments, the HP differentiation media comprise BMP4, FGF2 and VEGF-165. In some embodiments, the HP differentiation media comprises BMP4, FGF, VEGF-165 and a ROCK inhibitor. In some embodiments, the HP differentiation media comprises BMP4, FGF, VEGF-165 and Y27632.

In some embodiments, the HP differentiation media comprise 1-50 ng/mL BMP4, 5-50 ng/mL FGF2 and 1-100 ng/mL VEGF-165. In some embodiments, the HP differentiation media comprises 1-50 ng/mL BMP4, 1-50 ng/mL FGF, 1-100 ng/mL VEGF-165 and 1-20 μM of a ROCK inhibitor. In some embodiments, the HP differentiation media comprises 1-50 ng/mL BMP4, 1-50 ng/mL FGF, 1-100 ng/mL VEGF-165 and 1-20 μM Y27632.

In some embodiments, the HP differentiation media comprises a BMP pathway activator, a FGF, a VEGF, and a pyrimido-indole derivative. In some embodiments, the HP differentiation media comprises a BMP pathway activator, a FGF, a VEGF, and an aryl hydrocarbon receptor antagonist. In some embodiments, the HP differentiation media comprises a BMP pathway activator, a FGF, a VEGF, a pyrimido-indole derivative, and an aryl hydrocarbon receptor antagonist.

In some embodiments, the HP differentiation media comprises a BMP pathway activator, a FGF, a VEGF, SCF, TPO and an LDL. In some embodiments, the HP differentiation media comprises BMP4, FGF2, VEGF-165, SCF, TPO and LDL. In some embodiments, the HP differentiation media comprises 50 ng/mL BMP4, 5-50 ng/mL FGF2, 1-100 ng/mL VEGF-165, 1-100 ng/mL SCF, 1-100 ng/mL TPO, and 1-50 μg/mL LDL.

In some embodiments, the HP differentiation media comprises BMP4, FGF, VEGF-165, and a pyrimido-indole derivative. In some embodiments, the HP differentiation media comprises BMP4, FGF, VEGF-165, and an aryl hydrocarbon receptor antagonist. In some embodiments, the HP differentiation media comprises BMP4, FGF, VEGF-165, a pyrimido-indole derivative, and an aryl hydrocarbon receptor antagonist. In some embodiments, the HP differentiation media comprises BMP4, FGF, VEGF-165, and SR1. In some embodiments, the HP differentiation media comprises BMP4, FGF, VEGF-165, and UM729. In some embodiments, the HP differentiation media comprises BMP4, FGF, VEGF-165, UM729, and SR1.

In some embodiments, the HP differentiation media comprises 1-50 ng/mL BMP4, 1-50 ng/mL FGF, 1-100 ng/mL VEGF-165, and 0.1-10 μM UM729. In some embodiments, the HP differentiation media comprises 1-50 ng/mL BMP4, 1-50 ng/mL FGF, 1-100 ng/mL VEGF-165, and 0.1-10 μM SR1. In some embodiments, the HP differentiation media comprises 1-50 ng/mL BMP4, 1-50 ng/mL FGF, 1-100 ng/mL VEGF-165, 0.1-10 μM UM729, and 0.1-10 μM SR1.

In some embodiments, the HP differentiation media comprises a BMP pathway activator, a FGF, a VEGF, and a TGF-β receptor inhibitor. In some embodiments, the HP differentiation media comprises a BMP pathway activator, a FGF, a VEGF, a pyrimido-indole derivative, and a TGF-β receptor inhibitor. In some embodiments, the HP differentiation media comprises a BMP pathway activator, a FGF, a VEGF, an aryl hydrocarbon receptor antagonist, and a TGF-β receptor inhibitor. In some embodiments, the HP differentiation media comprises a BMP pathway activator, a FGF, a VEGF, a pyrimido-indole derivative, an aryl hydrocarbon receptor antagonist, and a TGF-β receptor inhibitor.

In some embodiments, the HP differentiation media comprises BMP4, FGF, VEGF-165, and a TGF-β receptor inhibitor. In some embodiments, the HP differentiation media comprises BMP4, FGF, VEGF-165, and GW788388. In some embodiments, the HP differentiation media comprises BMP4, FGF, VEGF-165, UM729, and GW788388. In some embodiments, the HP differentiation media comprises BMP4, FGF, VEGF-165, SR1, and GW788388. In some embodiments, the HP differentiation media comprises BMP4, FGF, VEGF-165, UM729, SR1, and GW788388.

In some embodiments, the HP differentiation media comprises 1-50 ng/mL BMP4, 1-50 ng/mL FGF, 1-100 ng/mL VEGF-165, and 0.1-20 μM of a TGF-β receptor inhibitor. In some embodiments, the HP differentiation media comprises 1-50 ng/mL BMP4, 1-50 ng/mL FGF, 1-100 ng/mL VEGF-165, and 0.1-20 μM GW788388. In some embodiments, the HP differentiation media comprises 1-50 ng/mL BMP4, 1-50 ng/mL FGF, 1-100 ng/mL VEGF-165, 0.1-10 μM UM729, and 0.1-20 μM GW788388. In some embodiments, the HP differentiation media comprises 1-50 ng/mL BMP4, 1-50 ng/mL FGF, 1-100 ng/mL VEGF-165, 0.1-10 μM SR1, and 0.1-20 μM GW788388. In some embodiments, the HP differentiation media comprises 1-50 ng/mL BMP4, 1-50 ng/mL FGF, 1-100 ng/mL VEGF-165, 0.1-10 μM UM729, and 0.1-10 μM SR1, and 0.1-20 μM GW788388.

In some embodiments, the HP differentiation media comprises BMP4, FGF, VEGF-165, and a TGF-β receptor inhibitor. In some embodiments, the HP differentiation media comprises BMP4, FGF, VEGF-165, and SB431542. In some embodiments, the HP differentiation media comprises BMP4, FGF, VEGF-165, UM729, and SB431542. In some embodiments, the HP differentiation media comprises BMP4, FGF, VEGF-165, SR1, and SB431542. In some embodiments, the HP differentiation media comprises BMP4, FGF, VEGF-165, UM729, SR1, and SB431542.

In some embodiments, the HP differentiation media comprises 1-50 ng/mL BMP4, 1-50 ng/mL FGF, 1-100 ng/mL VEGF-165, and 0.1-20 μM of a TGF-β receptor inhibitor. In some embodiments, the HP differentiation media comprises 1-50 ng/mL BMP4, 1-50 ng/mL FGF, 1-100 ng/mL VEGF-165, and 0.1-20 μM SB431542. In some embodiments, the HP differentiation media comprises 1-50 ng/mL BMP4, 1-50 ng/mL FGF, 1-100 ng/mL VEGF-165, 0.1-10 μM UM729, and 0.1-20 μM SB431542. In some embodiments, the HP differentiation media comprises 1-50 ng/mL BMP4, 1-50 ng/mL FGF, 1-100 ng/mL VEGF-165, 0.1-10 μM SR1, and 0.1-20 μM SB431542. In some embodiments, the HP differentiation media comprises 1-50 ng/mL BMP4, 1-50 ng/mL FGF, 1-100 ng/mL VEGF-165, 0.1-10 μM UM729, and 0.1-10 μM SR1, and 0.1-20 μM SB431542.

### NK Cell Differentiation Media

In some embodiments, the disclosure provides a differentiation media for generating NK cells from HP cells. In some embodiments, NK cells are generated from HP cells. In some embodiments, the HP cells produced by the compositions and methods of the disclosure are further differentiated to NK cells.

In some embodiments, a population of HP cells are cultured with at least one exogenous factor to form differentiated NK cells. In some embodiments, the exogenous factor is stem cell factor (SCF). In some embodiments, the exogenous factor is IL-7. In some embodiments, the exogenous factor is IL-15. In some embodiments, the exogenous factor is IL-12. In some embodiments, the exogenous factor is FLT3L. In some embodiments, the exogenous factor is a pyrimido-indole derivative. In some embodiments, the exogenous factor is an aryl hydrocarbon receptor antagonist. In some embodiments, the exogenous factors are selected from SCF, IL-7, IL-15, IL-12, a pyrimido-indole derivative, and an aryl hydrocarbon receptor antagonist.

In some embodiments, the exogenous factors comprise SCF and IL-7. In some embodiments, the exogenous factors comprise SCF and IL-15. In some embodiments, the exogenous factors comprise SCF and IL-12. In some embodiments, the exogenous factors comprise SCF and FLT3L. In some embodiments, the exogenous factors comprise SCF and pyrimido-indole derivative. In some embodiments, the exogenous factors comprise SCF and an aryl hydrocarbon receptor antagonist. In some embodiments, the exogenous factors comprise a IL-7 and a IL-15. In some embodiments, the exogenous factors comprise IL-7 and IL-12. In some embodiments, the exogenous factors comprise IL-7 and FLT3L. In some embodiments, the exogenous factors comprise IL-7 and pyrimido-indole derivative. In some embodiments, the exogenous factors comprise IL-7 and an aryl hydrocarbon receptor antagonist. In some embodiments, the exogenous factors comprise IL-15 and IL-12. In some embodiments, the exogenous factors comprise IL-15 and FLT3L. In some embodiments, the exogenous factors comprise IL-15 and pyrimido-indole derivative. In some embodiments, the exogenous factors comprise IL-15 and an aryl hydrocarbon receptor antagonist. In some embodiments, the exogenous factors comprise IL-12 and FLT3L. In some embodiments, the exogenous factors comprise IL-12 and pyrimido-indole derivative. In some embodiments, the exogenous factors comprise IL-12 and an aryl hydrocarbon receptor antagonist. In some embodiments, the exogenous factors comprise FLT3L and pyrimido-indole derivative. In some embodiments, the exogenous factors comprise FLT3L and an aryl hydrocarbon receptor antagonist. In some embodiments, the exogenous factors comprise a pyrimido-indole derivative and an aryl hydrocarbon receptor antagonist.

In some embodiments, the exogenous factors comprise SCF, IL-7, and IL-12. In some embodiments, the exogenous factors comprise SCF, IL-7, and IL-15. In some embodiments, the exogenous factors comprise SCF, IL-7, and FLT3L. In some embodiments, the exogenous factors comprise SCF, IL-7, and a pyrimido-indole derivative. In some embodiments, the exogenous factors comprise SCF, IL-7, and an aryl hydrocarbon receptor antagonist. In some embodiments, the exogenous factors comprise SCF, IL-12, and IL-15. In some embodiments, the exogenous factors comprise SCF, IL-12, and FLT3L. In some embodiments, the exogenous factors comprise SCF, IL-12, and a pyrimido-indole derivative. In some embodiments, the exogenous factors comprise SCF, IL-12, and an aryl hydrocarbon receptor antagonist. In some embodiments, the exogenous factors comprise SCF, IL-15, and FLT3L. In some embodiments, the exogenous factors comprise SCF, IL-15, and a pyrimido-indole derivative. In some embodiments, the exogenous factors comprise SCF, IL-15, and an aryl hydrocarbon receptor antagonist. In some embodiments, the exogenous factors comprise SCF, FLT3L, and a pyrimido-indole derivative. In some embodiments, the exogenous factors comprise SCF, FLT3L, and an aryl hydrocarbon receptor antagonist. In some embodiments, the exogenous factors comprise SCF, a pyrimido-indole derivative, and an aryl hydrocarbon receptor antagonist. In some embodiments, the exogenous factors comprise IL-7, IL-12, and IL-15. In some embodiments, the exogenous factors comprise IL-7, IL-12, and FLT3L. In some embodiments, the exogenous factors comprise IL-7, IL-12, and a pyrimido-indole derivative. In some embodiments, the exogenous factors comprise IL-7, IL-12, and a pyrimido-indole derivative. In some embodiments, the exogenous factors comprise IL-7, IL-15, and FLT3L. In some embodiments, the exogenous factors comprise IL-7, IL-15, and a pyrimido-indole derivative. In some embodiments, the exogenous factors comprise IL-7, IL-15, and an aryl hydrocarbon receptor antagonist. In some embodiments, the exogenous factors comprise IL-7, FLT3L, and a pyrimido-indole derivative. In some embodiments, the exogenous factors comprise IL-7, FLT3L, and an aryl hydrocarbon receptor antagonist. In some embodiments, the exogenous factors comprise IL-7, a pyrimido-indole derivative, and an aryl hydrocarbon receptor antagonist. In some embodiments, the exogenous factors comprise IL-12, IL-15, and FLT3L. In some embodiments, the exogenous factors comprise IL-12, IL-15, and a pyrimido-indole derivative. In some embodiments, the exogenous factors comprise IL-12, IL-15, and an aryl hydrocarbon receptor antagonist. In some embodiments, the exogenous factors comprise IL-12, FLT3L, and a pyrimido-indole derivative. In some embodiments, the exogenous factors comprise IL-12, FLT3L, and an aryl hydrocarbon receptor antagonist.

In some embodiments, the exogenous factors comprise SCF, IL-7, IL-12, and IL-15. In some embodiments, the exogenous factors comprise SCF, IL-7, IL-12, and FLT3L. In some embodiments, the exogenous factors comprise SCF, IL-7, IL-12, and a pyrimido-indole derivative. In some embodiments, the exogenous factors comprise SCF, IL-7, IL-12, and an aryl hydrocarbon receptor antagonist. In some embodiments, the exogenous factors comprise SCF, IL-7, IL-15, and FLT3L. In some embodiments, the exogenous factors comprise SCF, IL-7, IL-15, and a pyrimido-indole derivative. In some embodiments, the exogenous factors comprise SCF, IL-7, IL-15, and an aryl hydrocarbon receptor antagonist. In some embodiments, the exogenous factors comprise SCF, IL-7, FLT3L, and a pyrimido-indole derivative. In some embodiments, the exogenous factors comprise SCF, IL-7, FLT3L, and an aryl hydrocarbon receptor antagonist. In some embodiments, the exogenous factors comprise SCF, IL-7, a pyrimido-indole derivative, and an aryl hydrocarbon receptor antagonist. In some embodiments, the exogenous factors comprise SCF, IL-12, IL-15, and FLT3L. In some embodiments, the exogenous factors comprise SCF, IL-12, IL-15, and a pyrimido-indole derivative. In some embodiments, the exogenous factors comprise SCF, IL-12, IL-15, and an aryl hydrocarbon receptor antagonist. In some embodiments, the exogenous factors comprise SCF, IL-12, FLT3L, and a pyrimido-indole derivative. In some embodiments, the exogenous factors comprise SCF, IL-12, FLT3L, and an aryl hydrocarbon receptor antagonist. In some embodiments, the exogenous factors comprise SCF, IL-12, a pyrimido-indole derivative, and an aryl hydrocarbon receptor antagonist. In some embodiments, the exogenous factors comprise SCF, IL-15, FLT3L, and a pyrimido-indole derivative. In some embodiments, the exogenous factors comprise SCF, IL-15, FLT3L, and an aryl hydrocarbon receptor antagonist. In some embodiments, the exogenous factors comprise SCF, IL-15, FLT3L, and an aryl hydrocarbon receptor antagonist. In some embodiments, the exogenous factors comprise SCF, FLT3L, a pyrimido-indole derivative, and an aryl hydrocarbon receptor antagonist. In some embodiments, the exogenous factors comprise IL-7, IL-12, IL-15, and FLT3L. In some embodiments, the exogenous factors comprise IL-7, IL-12, IL-15, and a pyrimido-indole derivative. In some embodiments, the exogenous factors comprise IL-7, IL-12, IL-15, and an aryl hydrocarbon receptor antagonist. In some embodiments, the exogenous factors comprise IL-7, IL-12, FLT3L, and a pyrimido-indole derivative. In some embodiments, the exogenous factors comprise IL-7, IL-12, FLT3L, and an aryl hydrocarbon receptor antagonist. In some embodiments, the exogenous factors comprise IL-7, IL-12, a pyrimido-indole derivative, and an aryl hydrocarbon receptor antagonist. In some embodiments, the exogenous factors comprise IL-7, IL-15, FLT3L, and a pyrimido-indole derivative. In some embodiments, the exogenous factors comprise IL-7, IL-15, FLT3L, and an aryl hydrocarbon receptor antagonist. In some embodiments, the exogenous factors comprise IL-7, IL-15, a pyrimido-indole derivative, and an aryl hydrocarbon receptor antagonist. In some embodiments, the exogenous factors comprise IL-7, FLT3L, a pyrimido-indole derivative, and an aryl hydrocarbon receptor antagonist.

In some embodiments, the exogenous factors comprise SCF, IL-7, IL-12, IL-15, and FLT3L. In some embodiments, the exogenous factors comprise SCF, IL-7, IL-12, 11-15, and a pyrimido-indole derivative. In some embodiments, the exogenous factors comprise SCF, IL-7, IL-12, IL-15, and an aryl hydrocarbon receptor antagonist. In some embodiments, the exogenous factors comprise SCF, IL-7, IL-12, FLT3L, and a pyrimido-indole derivative. In some embodiments, the exogenous factors comprise SCF, IL-7, IL-12, FLT3L, and an aryl hydrocarbon receptor antagonist. In some embodiments, the exogenous factors comprise SCF, IL-7, IL-12, a pyrimido-indole derivative, and an aryl hydrocarbon receptor antagonist. In some embodiments, the exogenous factors comprise SCF, IL-7, IL-15, FLT3L, and a pyrimido-indole derivative. In some embodiments, the exogenous factors comprise SCF, IL-7, IL-15, FLT3L, and an aryl hydrocarbon receptor antagonist. In some embodiments, the exogenous factors comprise SCF, IL-7, IL-15, a pyrimido-indole derivative, and an aryl hydrocarbon receptor antagonist. In some embodiments, the exogenous factors comprise SCF, IL-7, FLT3L, a pyrimido-indole derivative, and an aryl hydrocarbon receptor antagonist. In some embodiments, the exogenous factors comprise SCF, IL-12, IL-15, FLT3L, and a pyrimido-indole derivative. In some embodiments, the exogenous factors comprise SCF, IL-12, IL-15, FLT3L, and an aryl hydrocarbon receptor antagonist. In some embodiments, the exogenous factors comprise SCF, IL-12, IL-15, a pyrimido-indole derivative, and an aryl hydrocarbon receptor antagonist. In some embodiments, the exogenous factors comprise SCF, IL-12, FLT3L, a pyrimido-indole derivative, and an aryl hydrocarbon receptor antagonist. In some embodiments, the exogenous factors comprise SCF, IL-15, FLT3L, a pyrimido-indole derivative, and an aryl hydrocarbon receptor antagonist. In some embodiments, the exogenous factors comprise IL-7, IL-12, IL-15, FLT3L, and a pyrimido-indole derivative. In some embodiments, the exogenous factors comprise IL-7, IL-12, IL-15, FLT3L, and an aryl hydrocarbon receptor antagonist. In some embodiments, the exogenous factors comprise IL-7, IL-12, IL-15, a pyrimido-indole derivative, and an aryl hydrocarbon receptor antagonist. In some embodiments, the exogenous factors comprise IL-7, IL-12, FLT3L, a pyrimido-indole derivative, and an aryl hydrocarbon receptor antagonist. In some embodiments, the exogenous factors comprise IL-7, IL-12, IL-15, FLT3L, and a pyrimido-indole derivative. In some embodiments, the exogenous factors comprise IL-7, IL-12, IL-15, FLT3L, and an aryl hydrocarbon receptor antagonist. In some embodiments, the exogenous factors comprise IL-7, IL-12, IL-15, a pyrimido-indole derivative, and an aryl hydrocarbon receptor antagonist. In some embodiments, the exogenous factors comprise IL-7, IL-12, FLT3L, a pyrimido-indole derivative, and an aryl hydrocarbon receptor antagonist. In some embodiments, the exogenous factors comprise IL-7, IL-15, FLT3L, a pyrimido-indole derivative, and an aryl hydrocarbon receptor antagonist. In some embodiments, the exogenous factors comprise IL-12, IL-15, FLT3L, a pyrimido-indole derivative, and an aryl hydrocarbon receptor antagonist.

In some embodiments, the exogenous factors comprise SCF, IL-7, IL-12, IL-15, FLT3L, and a pyrimido-indole derivative. In some embodiments, the exogenous factors comprise SCF, IL-7, IL-12, IL-15, FLT3L, and an aryl hydrocarbon receptor antagonist. In some embodiments, the exogenous factors comprise SCF, IL-7, IL-12, IL-15, a pyrimido-indole derivative, and an aryl hydrocarbon receptor antagonist. In some embodiments, the exogenous factors comprise SCF, IL-7, IL-12, FLT3L, a pyrimido-indole derivative, and an aryl hydrocarbon receptor antagonist. In some embodiments, the exogenous factors comprise SCF, IL-7, IL-15, FLT3L, a pyrimido-indole derivative, and an aryl hydrocarbon receptor antagonist. In some embodiments, the exogenous factors comprise SCF, IL-12, IL-15, FLT3L, a pyrimido-indole derivative, and an aryl hydrocarbon receptor antagonist. In some embodiments, the exogenous factors comprise IL-7, IL-12, IL-15, FLT3L, a pyrimido-indole derivative, and an aryl hydrocarbon receptor antagonist.

In some embodiments, the exogenous factors comprise SCF, IL-7, and IL-12. In some embodiments, the exogenous factors comprise SCF, IL-7, and IL-15. In some embodiments, the exogenous factors comprise SCF, IL-7, and FLT3L. In some embodiments, the exogenous factors comprise SCF, IL-7, and a pyrimido-indole derivative. In some embodiments, the exogenous factors comprise SCF, IL-7, and an aryl hydrocarbon receptor antagonist. In some embodiments, the exogenous factors comprise SCF, IL-12, and IL-15. In some embodiments, the exogenous factors comprise SCF, IL-12, and FLT3L. In some embodiments, the exogenous factors comprise SCF, IL-12, and a pyrimido-indole derivative. In some embodiments, the exogenous factors comprise SCF, IL-12, and SR1. In some embodiments, the exogenous factors comprise SCF, IL-15, and FLT3L. In some embodiments, the exogenous factors comprise SCF, IL-15, and a pyrimido-indole derivative. In some embodiments, the exogenous factors comprise SCF, IL-15, and SR1. In some embodiments, the exogenous factors comprise SCF, FLT3L, and a pyrimido-indole derivative. In some embodiments, the exogenous factors comprise SCF, FLT3L, and SR1. In some embodiments, the exogenous factors comprise SCF, a pyrimido-indole derivative, and SR1. In some embodiments, the exogenous factors comprise IL-7, IL-12, and IL-15. In some embodiments, the exogenous factors comprise IL-7, IL-12, and FLT3L. In some embodiments, the exogenous factors comprise IL-7, IL-12, and a pyrimido-indole derivative. In some embodiments, the exogenous factors comprise IL-7, IL-12, and a pyrimido-indole derivative. In some embodiments, the exogenous factors comprise IL-7, IL-15, and FLT3L. In some embodiments, the exogenous factors comprise IL-7, IL-15, and a pyrimido-indole derivative. In some embodiments, the exogenous factors comprise IL-7, IL-15, and SR1. In some embodiments, the exogenous factors comprise IL-7, FLT3L, and a pyrimido-indole derivative. In some embodiments, the exogenous factors comprise IL-7, FLT3L, and SR1. In some embodiments, the exogenous factors comprise IL-7, UM729, and SR1. In some embodiments, the exogenous factors comprise IL-12, IL-15, and FLT3L. In some embodiments, the exogenous factors comprise IL-12, IL-15, and UM729. In some embodiments, the exogenous factors comprise IL-12, IL-15, and SR1. In some embodiments, the exogenous factors comprise IL-12, FLT3L, and UM729. In some embodiments, the exogenous factors comprise IL-12, FLT3L, and SR1.

In some embodiments, the exogenous factors comprise SCF, IL-7, IL-12, and IL-15. In some embodiments, the exogenous factors comprise SCF, TL-7, IL-12, and FLT3L. In some embodiments, the exogenous factors comprise SCF, IL-7, IL-12, and UM729. In some embodiments, the exogenous factors comprise SCF, IL-7, IL-12, and SR1. In some embodiments, the exogenous factors comprise SCF, IL-7, IL-15, and FLT3L. In some embodiments, the exogenous factors comprise SCF, IL-7, IL-15, and UM729. In some embodiments, the exogenous factors comprise SCF, IL-7, IL-15, and SR1. In some embodiments, the exogenous factors comprise SCF, IL-7, FLT3L, and UM729. In some embodiments, the exogenous factors comprise SCF, IL-7, FLT3L, and SR1. In some embodiments, the exogenous factors comprise SCF, IL-7, UM729, and SR1. In some embodiments, the exogenous factors comprise SCF, IL-12, IL-15, and FLT3L. In some embodiments, the exogenous factors comprise SCF, IL-12, IL-15, and UM729. In some embodiments, the exogenous factors comprise SCF, IL-12, IL-15, and SR1. In some embodiments, the exogenous factors comprise SCF, IL-12, FLT3L, and UM729. In some embodiments, the exogenous factors comprise SCF, IL-12, FLT3L, and SR1. In some embodiments, the exogenous factors comprise SCF, IL-12, UM729, and SR1. In some embodiments, the exogenous factors comprise SCF, IL-15, FLT3L, and UM729. In some embodiments, the exogenous factors comprise SCF, IL-15, FLT3L, and SR1. In some embodiments, the exogenous factors comprise SCF, IL-15, FLT3L, and SR1. In some embodiments, the exogenous factors comprise SCF, FLT3L, UM729, and SR1. In some embodiments, the exogenous factors comprise IL-7, IL-12, IL-15, and FLT3L. In some embodiments, the exogenous factors comprise IL-7, IL-12, IL-15, and UM729. In some embodiments, the exogenous factors comprise IL-7, IL-12, IL-15, and SR1. In some embodiments, the exogenous factors comprise IL-7, IL-12, FLT3L, and UM729. In some embodiments, the exogenous factors comprise IL-7, IL-12, FLT3L, and SR1. In some embodiments, the exogenous factors comprise IL-7, IL-12, UM729, and SR1. In some embodiments, the exogenous factors comprise IL-7, IL-15, FLT3L, and UM729. In some embodiments, the exogenous factors comprise IL-7, IL-15, FLT3L, and SR1. In some embodiments, the exogenous factors comprise IL-7, IL-15, UM729, and SR1. In some embodiments, the exogenous factors comprise IL-7, FLT3L, UM729, and SR1.

In some embodiments, the exogenous factors comprise SCF, IL-7, IL-12, IL-15, and FLT3L. In some embodiments, the exogenous factors comprise SCF, IL-7, IL-12, IL-15, and UM729. In some embodiments, the exogenous factors comprise SCF, IL-7, IL-12, IL-15, and SR1. In some embodiments, the exogenous factors comprise SCF, IL-7, IL-12, FLT3L, and UM729. In some embodiments, the exogenous factors comprise SCF, IL-7, IL-12, FLT3L, and SR1. In some embodiments, the exogenous factors comprise SCF, IL-7, IL-12, UM729, and SR1. In some embodiments, the exogenous factors comprise SCF, IL-7, IL-15, FLT3L, and UM729. In some embodiments, the exogenous factors comprise SCF, IL-7, IL-15, FLT3L, and SR1. In some embodiments, the exogenous factors comprise SCF, IL-7, IL-15, UM729, and SR1. In some embodiments, the exogenous factors comprise SCF, IL-7, FLT3L, UM729, and SR1. In some embodiments, the exogenous factors comprise SCF, IL-12, IL-15, FLT3L, and UM729. In some embodiments, the exogenous factors comprise SCF, IL-12, IL-15, FLT3L, and SR1. In some embodiments, the exogenous factors comprise SCF, IL-12, IL-15, UM729, and SR1. In some embodiments, the exogenous factors comprise SCF, IL-12, FLT3L, UM729, and SR1. In some embodiments, the exogenous factors comprise SCF, IL-15, FLT3L, UM729, and SR1. In some embodiments, the exogenous factors comprise IL-7, IL-12, IL-15, FLT3L, and UM729. In some embodiments, the exogenous factors comprise IL-7, IL-12, IL-15, FLT3L, and SR1. In some embodiments, the exogenous factors comprise IL-7, IL-12, IL-15, UM729, and SR1. In some embodiments, the exogenous factors comprise IL-7, IL-12, FLT3L, UM729, and SR1. In some embodiments, the exogenous factors comprise IL-7, IL-12, IL-15, FLT3L, and UM729. In some embodiments, the exogenous factors comprise IL-7, IL-12, IL-15, FLT3L, and SR1. In some embodiments, the exogenous factors comprise IL-7, IL-12, IL-15, UM729, and SR1. In some embodiments, the exogenous factors comprise IL-7, IL-12, FLT3L, UM729, and SR1. In some embodiments, the exogenous factors comprise IL-7, IL-15, FLT3L, UM729, and SR1. In some embodiments, the exogenous factors comprise IL-12, IL-15, FLT3L, UM729, and SR1.

In some embodiments, the exogenous factors comprise SCF, IL-7, IL-12, IL-15, FLT3L, and UM729. In some embodiments, the exogenous factors comprise SCF, IL-7, IL-12, IL-15, FLT3L, and SR1. In some embodiments, the exogenous factors comprise SCF, IL-7, IL-12, IL-15, UM729, and SR1. In some embodiments, the exogenous factors comprise SCF, IL-7, IL-12, FLT3L, UM729, and SR1. In some embodiments, the exogenous factors comprise SCF, IL-7, IL-15, FLT3L, UM729, and SR1. In some embodiments, the exogenous factors comprise SCF, IL-12, IL-15, FLT3L, UM729, and SR1. In some embodiments, the exogenous factors comprise IL-7, IL-12, IL-15, FLT3L, UM729, and SR1.

In some embodiments, SCF is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, SCF is present in differentiation media at about 1-50 ng/ml.

In some embodiments, IL-7 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-7 is present in differentiation media at about 1-50 ng/ml.

In some embodiments, IL-12 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-12 is present in differentiation media at about 1-100 ng/ml.

In some embodiments, IL-15 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-15 is present in differentiation media at about 1-100 ng/ml.

In some embodiments, FLT3L is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, FLT3L is present in differentiation media at about 1-100 ng/ml.

In some embodiments, the pyrimido-indole derivative is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, the pyrimido-indole derivative is present in differentiation media at about 0.1-10 μM.

In some embodiments, the pyrimido-indole derivative is UM729. In some embodiments, UM729 is present at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, about or 100 μM, or any range derivable therein. In some embodiments, UM729 is present in differentiation media at about 0.1-10 μM.

In some embodiments, the aryl hydrocarbon receptor antagonist is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, the aryl hydrocarbon receptor antagonist is present in differentiation media at about 0.1-10 μM.

In some embodiments, the aryl hydrocarbon receptor antagonist is StemRegenin 1 (SR1). In some embodiments, SR1 is present at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, about or 100 μM, or any range derivable therein. In some embodiments, StemRegenin 1 (SR1) is present in differentiation media at about 0.1-10 μM.

In some embodiments, the NK cell differentiation media comprises SCF, IL-7, IL-12, IL-15, and FLT3L. In some embodiments, the NK cell differentiation media comprises SCF, IL-7, IL-12, IL-15, FLT3L, and a pyrimido-indole derivative. In some embodiments, the NK cell differentiation media comprises SCF, IL-7, IL-12, IL-15, FLT3L, and an aryl hydrocarbon receptor antagonist. In some embodiments, the NK cell differentiation media comprises SCF, IL-7, IL-12, IL-15, FLT3L, a pyrimido-indole derivative, and an aryl hydrocarbon receptor antagonist.

In some embodiments, the NK cell differentiation media comprises SCF, IL-7, IL-12, IL-15, FLT3L, and SR1. In some embodiments, the NK cell differentiation media comprises SCF, IL-7, IL-12, IL-15, FLT3L, and UM729. In some embodiments, the NK cell differentiation media comprises SCF, IL-7, IL-12, IL-15, FLT3L, SR1, and UM729.

In some embodiments, the NK cell differentiation media comprises 1-50 ng/ml SCF, 1-50 ng/ml IL-7, 1-100 ng/ml IL-12, 1-100 ng/ml IL-15, and 1-100 ng/ml FLT3L. In some embodiments, the NK cell differentiation media comprises 1-50 ng/ml SCF, 1-50 ng/ml IL-7, 1-100 ng/ml IL-12, 1-100 ng/ml IL-15, 1-100 ng/ml FLT3L, and 0.1-10 μM of a pyrimido-indole derivative. In some embodiments, the NK cell differentiation media comprises 1-50 ng/ml SCF, 1-50 NG/ML IL-7, 1-100 ng/ml IL-12, 1-100 ng/ml IL-15, 1-100 ng/ml FLT3L, and 0.1-10 μM of an aryl hydrocarbon receptor antagonist. In some embodiments, the NK cell differentiation media comprises 1-50 ng/ml SCF, 1-50 ng/ml IL-7, 1-100 ng/ml IL-12, 1-100 ng/ml IL-15, 1-100 ng/ml FLT3L, 0.1-10 μM of a pyrimido-indole derivative, and 0.1-10 μM of an aryl hydrocarbon receptor antagonist.

In some embodiments, the NK cell differentiation media comprises 1-50 ng/ml SCF, 1-50 ng/ml IL-7, 1-100 ng/ml IL-12, 1-100 ng/ml IL-15, 1-100 ng/ml FLT3L, and SR1. In some embodiments, the NK cell differentiation media comprises 1-50 ng/ml SCF, 1-50 ng/ml IL-7, 1-100 ng/ml IL-12, 1-100 ng/ml IL-15, 1-100 ng/ml FLT3L, and 0.1-10 μM UM729. In some embodiments, the NK cell differentiation media comprises 1-50 ng/ml SCF, 1-50 ng/ml IL-7, 1-100 ng/ml IL-12, 1-100 ng/ml IL-15, 1-100 ng/ml FLT3L, 1-10 μM SR1, and 0.1-10 μM UM729.

In some embodiments, the NK cell differentiation media comprises a defined xenogenic-free base media, 1-50 ng/ml SCF, 1-50 ng/ml IL-7, 1-100 ng/ml IL-12, 1-100 ng/ml IL-15, 1-100 ng/ml FLT3L, and 0.1-10 μM SR1. In some embodiments, the NK cell differentiation media comprises a defined xenogenic-freebase media, 1-50 ng/ml SCF, 1-50 ng/ml IL-7, 1-100 ng/ml IL-12, 1-100 ng/ml IL-15, 1-100 ng/ml FLT3L, and 0.1-10 μM UM729. In some embodiments, the NK cell differentiation media comprises a defined xenogenic-freebase media, 1-50 ng/ml SCF, 1-50 ng/ml IL-7, 1-100 ng/ml IL-12, 1-100 ng/ml IL-15, 1-100 ng/ml FLT3L, a 0.1-10 μM SR1, and a 0.1-10 μM UM729.

### NK Cell Expansion Media

In some embodiments, the disclosure provides an expansion media for generating mature NK cells from differentiated NK cells. In some embodiments, differentiated NK cells are generated from HP cells. In some embodiments, the differentiated NK cells produced by the compositions and methods of the disclosure are further expanded to mature NK cells.

In some embodiments, a population of differentiated NK cells are cultured with at least one exogenous factor to form mature NK cells. In some embodiments, the exogenous factor is IL-2. In some embodiments, the exogenous factor is exogenous factor is IL-7. In some embodiments, the exogenous factor is IL-12. In some embodiments, the exogenous factor is IL-15. In some embodiments, the exogenous factor is IL-18. In some embodiments, the exogenous factor is LDL. In some embodiments, the exogenous factor is activation beads. In some embodiments, the exogenous factors are selected from IL-2, IL-7, IL-12, IL-15, IL-18, and activation beads.

In some embodiments, the exogenous factors comprise IL-2 and IL-7. In some embodiments, the exogenous factors comprise IL-2 and IL-12. In some embodiments, the exogenous factors comprise IL-2 and IL-15. In some embodiments, the exogenous factors comprise IL-2 and IL-18. In some embodiments, the exogenous factors comprise IL-2 and activation beads. In some embodiments, the exogenous factors comprise IL-7 and IL-12. In some embodiments, the exogenous factors comprise IL-7 and IL-15. In some embodiments, the exogenous factors comprise IL-7 and IL-18. In some embodiments, the exogenous factors comprise IL-7 and activation beads. In some embodiments, the exogenous factors comprise IL-12 and IL-15. In some embodiments, the exogenous factors comprise IL-12 and IL-18. In some embodiments, the exogenous factors comprise IL-12 and activation beads. In some embodiments, the exogenous factors comprise IL-15 and IL-18. In some embodiments, the exogenous factors comprise IL-15 and activation beads. In some embodiments, the exogenous factors comprise IL-18 and activation beads.

In some embodiments, the exogenous factors comprise IL-2, IL-7, and IL-12. In some embodiments, the exogenous factors comprise IL-2, IL-7, and IL-15. In some embodiments, the exogenous factors comprise IL-2, IL-7, and IL-18. In some embodiments, the exogenous factors comprise IL-2, IL-7, and activation beads. some embodiments, the exogenous factors comprise IL-2, IL-12, and IL-15. In some embodiments, the exogenous factors comprise IL-2, IL-12, and IL-18. In some embodiments, the exogenous factors comprise IL-2, IL-12, and activation beads. In some embodiments, the exogenous factors comprise IL-2, IL-15, and IL-18. In some embodiments, the exogenous factors comprise IL-2, IL-15, and activation beads. In some embodiments, the exogenous factors comprise IL-2, IL-18, and activation beads. In some embodiments, the exogenous factors comprise IL-7, IL-12, and IL-15. In some embodiments, the exogenous factors comprise IL-7, IL-12, and IL-18. In some embodiments, the exogenous factors comprise IL-7, IL-12, and activation beads. In some embodiments, the exogenous factors comprise IL-7, IL-15, and IL-18. In some embodiments, the exogenous factors comprise IL-7, IL-15, and activation beads. In some embodiments, the exogenous factors comprise IL-7, IL-18, and activation beads. In some embodiments, the exogenous factors comprise IL-12, IL-15, and IL-18. In some embodiments, the exogenous factors comprise IL-12, IL-15, and activation beads. In some embodiments, the exogenous factors comprise IL-12, IL-18, and activation beads. In some embodiments, the exogenous factors comprise IL-15, IL-18, and activation beads.

In some embodiments, the exogenous factors comprise IL-2, IL-7, IL-12, and IL-15. In some embodiments, the exogenous factors comprise IL-2, IL-7, IL-12, and IL-18. In some embodiments, the exogenous factors comprise IL-2, IL-7, IL-12, and activation beads. In some embodiments, the exogenous factors comprise IL-2, IL-7, IL-15, and IL-18. In some embodiments, the exogenous factors comprise IL-2, IL-7, IL-15, and activation beads. In some embodiments, the exogenous factors comprise IL-2, IL-7, IL-18, and activation beads. In some embodiments, the exogenous factors comprise IL-2, IL-12, IL-15, and IL-18. In some embodiments, the exogenous factors comprise IL-2, IL-12, IL-15, and activation beads. In some embodiments, the exogenous factors comprise IL-2, IL-12, IL-18, and activation beads. In some embodiments, the exogenous factors comprise IL-2, IL-15, IL-18, and activation beads. In some embodiments, the exogenous factors comprise IL-7, IL-12, IL-15, and IL-18. In some embodiments, the exogenous factors comprise IL-7, IL-12, IL-15, and activation beads. In some embodiments, the exogenous factors comprise IL-7, IL-12, IL-18, and activation beads. In some embodiments, the exogenous factors comprise IL-7, IL-15, IL-18, and activation beads. In some embodiments, the exogenous factors comprise IL-12, IL-15, IL-18, and activation beads.

In some embodiments, the exogenous factors comprise IL-2, IL-7, IL-12, IL-15, and IL-18. In some embodiments, the exogenous factors comprise IL-2, IL-7, IL-12, IL-15, and activation beads. In some embodiments, the exogenous factors comprise IL-2, IL-7, IL-12, IL-18, and activation beads. In some embodiments, the exogenous factors comprise IL-2, IL-7, IL-15, IL-18, and activation beads. In some embodiments, the exogenous factors comprise IL-2, IL-12, IL-15, IL-18, and activation beads. In some embodiments, the exogenous factors comprise IL-7, IL-12, IL-15, IL-18, and activation beads.

In some embodiments, the exogenous factors comprise IL-2, IL-7, IL-12, IL-15, IL-18, and activation beads.

In some embodiments, the exogenous factors comprise IL-2 and IL-7. In some embodiments, the exogenous factors comprise IL-2 and IL-12. In some embodiments, the exogenous factors comprise IL-2 and IL-15. In some embodiments, the exogenous factors comprise IL-2 and IL-18. In some embodiments, the exogenous factors comprise IL-2 and activation beads coated with anti-CD2/anti-NKp46. In some embodiments, the exogenous factors comprise IL-7 and IL-12. In some embodiments, the exogenous factors comprise IL-7 and IL-15. In some embodiments, the exogenous factors comprise IL-7 and IL-18. In some embodiments, the exogenous factors comprise IL-7 and activation beads coated with anti-CD2/anti-NKp46. In some embodiments, the exogenous factors comprise IL-12 and IL-15. In some embodiments, the exogenous factors comprise IL-12 and IL-18. In some embodiments, the exogenous factors comprise IL-12 and activation beads coated with anti-CD2/anti-NKp46. In some embodiments, the exogenous factors comprise IL-15 and IL-18. In some embodiments, the exogenous factors comprise IL-15 and activation beads coated with anti-CD2/anti-NKp46. In some embodiments, the exogenous factors comprise IL-18 and activation beads coated with anti-CD2/anti-NKp46.

In some embodiments, the exogenous factors comprise IL-2, IL-7, and IL-12. In some embodiments, the exogenous factors comprise IL-2, IL-7, and IL-15. In some embodiments, the exogenous factors comprise IL-2, IL-7, and IL-18. In some embodiments, the exogenous factors comprise IL-2, IL-7, and activation beads coated with anti-CD2/anti-NKp46. some embodiments, the exogenous factors comprise IL-2, IL-12, and IL-15. In some embodiments, the exogenous factors comprise IL-2, IL-12, and IL-18. In some embodiments, the exogenous factors comprise IL-2, IL-12, and activation beads coated with anti-CD2/anti-NKp46. In some embodiments, the exogenous factors comprise IL-2, IL-15, and IL-18. In some embodiments, the exogenous factors comprise IL-2, IL-15, and activation beads coated with anti-CD2/anti-NKp46. In some embodiments, the exogenous factors comprise IL-2, IL-18, and activation beads coated with anti-CD2/anti-NKp46. In some embodiments, the exogenous factors comprise IL-7, IL-12, and IL-15. In some embodiments, the exogenous factors comprise IL-7, IL-12, and IL-18. In some embodiments, the exogenous factors comprise IL-7, IL-12, and activation beads coated with anti-CD2/anti-NKp46. In some embodiments, the exogenous factors comprise IL-7, IL-15, and IL-18. In some embodiments, the exogenous factors comprise IL-7, IL-15, and activation beads coated with anti-CD2/anti-NKp46. In some embodiments, the exogenous factors comprise IL-7, IL-18, and activation beads coated with anti-CD2/anti-NKp46. In some embodiments, the exogenous factors comprise IL-12, IL-15, and IL-18. In some embodiments, the exogenous factors comprise IL-12, IL-15, and activation beads coated with anti-CD2/anti-NKp46. In some embodiments, the exogenous factors comprise IL-12, IL-18, and activation beads coated with anti-CD2/anti-NKp46. In some embodiments, the exogenous factors comprise IL-15, IL-18, and activation beads coated with anti-CD2/anti-NKp46.

In some embodiments, the exogenous factors comprise IL-2, IL-7, IL-12, and IL-15. In some embodiments, the exogenous factors comprise IL-2, IL-7, IL-12, and IL-18. In some embodiments, the exogenous factors comprise IL-2, IL-7, IL-12, and activation beads coated with anti-CD2/anti-NKp46. In some embodiments, the exogenous factors comprise IL-2, IL-7, IL-15, and IL-18. In some embodiments, the exogenous factors comprise IL-2, IL-7, IL-15, and activation beads coated with anti-CD2/anti-NKp46. In some embodiments, the exogenous factors comprise IL-2, IL-7, IL-18, and activation beads coated with anti-CD2/anti-NKp46. In some embodiments, the exogenous factors comprise IL-2, IL-12, IL-15, and IL-18. In some embodiments, the exogenous factors comprise IL-2, IL-12, IL-15, and activation beads coated with anti-CD2/anti-NKp46. In some embodiments, the exogenous factors comprise IL-2, IL-12, IL-18, and activation beads coated with anti-CD2/anti-NKp46. In some embodiments, the exogenous factors comprise IL-2, IL-15, IL-18, and activation beads coated with anti-CD2/anti-NKp46. In some embodiments, the exogenous factors comprise IL-7, IL-12, IL-15, and IL-18. In some embodiments, the exogenous factors comprise IL-7, IL-12, IL-15, and activation beads coated with anti-CD2/anti-NKp46. In some embodiments, the exogenous factors comprise IL-7, IL-12, IL-18, and activation beads coated with anti-CD2/anti-NKp46. In some embodiments, the exogenous factors comprise IL-7, IL-15, IL-18, and activation beads coated with anti-CD2/anti-NKp46. In some embodiments, the exogenous factors comprise IL-12, IL-15, IL-18, and activation beads coated with anti-CD2/anti-NKp46.

In some embodiments, the exogenous factors comprise IL-2, IL-7, IL-12, IL-15, and IL-18. In some embodiments, the exogenous factors comprise IL-2, IL-7, IL-12, IL-15, and activation beads coated with anti-CD2/anti-NKp46. In some embodiments, the exogenous factors comprise IL-2, IL-7, IL-12, IL-18, and activation beads coated with anti-CD2/anti-NKp46. In some embodiments, the exogenous factors comprise IL-2, IL-7, IL-15, IL-18, and activation beads coated with anti-CD2/anti-NKp46. In some embodiments, the exogenous factors comprise IL-2, IL-12, IL-15, IL-18, and activation beads coated with anti-CD2/anti-NKp46. In some embodiments, the exogenous factors comprise IL-7, IL-12, IL-15, IL-18, and activation beads coated with anti-CD2/anti-NKp46.

In some embodiments, the exogenous factors comprise IL-2, IL-7, IL-12, IL-15, IL-18, and activation beads coated with anti-CD2/anti-NKp46.

In some embodiments, IL-2 is present in the expansion media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-2 is present in expansion media at about 1-50 ng/ml.

In some embodiments, IL-7 is present in the expansion media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-7 is present in expansion media at about 1-50 ng/ml.

In some embodiments, IL-12 is present in the expansion media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-12 is present in expansion media at about 1-100 ng/ml.

In some embodiments, IL-15 is present in the expansion media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-15 is present in expansion media at about 1-100 ng/ml.

In some embodiments, IL-18 is present in the expansion media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-18 is present in expansion media at about 1-100 ng/ml.

In some embodiments, activation beads are present in the expansion media.

In some embodiments, activation beads coated with anti-CD2/anti-NKp46 are present in the expansion media at a ratio based on the number of differentiated NK cells. For example, one activation bead is present for each NK cell, or a ratio of 1:1 activation bead to NK cell.

In some embodiments, the activation bead:NK cell ratio is at least 1:1, at least 2:1, at least 3:1, at least 4:1, at least 5:1, at least 6:1, at least 7:1, at least 8:1, at least 9:1, at least 10:1, at least 15:1, at least 20:1, at least 25:1, at least 30:1, at least 35:1, at least 40:1, at least 45:1, or at least 50:1.

In some embodiments, the activation bead:NK cell ratio is about 1:1 to 2:1, about 2:1 to 3:1, about 3:1 to 4:1, about 4:1 to 5:1, about 5:1 to 6:1, about 6:1 to 7:1, about 7:1 to 8:1, about 8:1 to 9:1, about 9:1 to 10:1, about 10:1 to 15:1, about 15:1 to 20:1, about 20:1 to 25:1, about 25:1 to 30:1, about 30:1 to 35:1, about 35:1 to 40:1, about 40:1 to 45:1, or about 45:1 to 50:1.

In some embodiments, the NK cell:activation bead ratio is at least 1:1, at least 2:1, at least 3:1, at least 4:1, at least 5:1, at least 6:1, at least 7:1, at least 8:1, at least 9:1, at least 10:1, at least 15:1, at least 20:1, at least 25:1, at least 30:1, at least 35:1, at least 40:1, at least 45:1, or at least 50:1.

In some embodiments, the NK cell:activation bead ratio is about 1:1 to 2:1, about 2:1 to 3:1, about 3:1 to 4:1, about 4:1 to 5:1, about 5:1 to 6:1, about 6:1 to 7:1, about 7:1 to 8:1, about 8:1 to 9:1, about 9:1 to 10:1, about 10:1 to 15:1, about 15:1 to 20:1, about 20:1 to 25:1, about 25:1 to 30:1, about 30:1 to 35:1, about 35:1 to 40:1, about 40:1 to 45:1, or about 45:1 to 50:1.

In some embodiments, the NK cell expansion media comprises IL-2, IL-7, IL-12, IL-15, IL-18, and activation beads. In some embodiments, the NK cell expansion media comprises a defined xenogenic-freebase media, comprises IL-2, IL-7, IL-12, IL-15, IL-18, and activation beads.

In some embodiments, the NK cell expansion media comprises IL-2, IL-7, IL-12, IL-15, IL-18, and activation beads coated with anti-CD2/anti-NKp46. In some embodiments, the NK cell expansion media comprises a defined xenogenic-free base media, comprises IL-2, IL-7, IL-12, IL-15, IL-18, and activation beads coated with anti-CD2/anti-NKp46.

In some embodiments, the NK cell expansion media comprises 1-50 ng/ml IL-2, 1-50 ng/ml IL-7, 1-100 ng/ml IL-12, 1-100 ng/ml IL-15, 1-100 ng/ml IL-18, and activation beads at a cell:bead ratio of 1:1. In some embodiments, the NK cell expansion media comprises a defined xenogenic-freebase media, comprises 1-50 ng/ml IL-2, 1-50 ng/ml IL-7, 1-100 ng/ml IL-12, 1-100 ng/ml IL-15, 1-100 ng/ml IL-18, and activation beads at a cell:bead ratio of 1:1.

In some embodiments, the NK cell expansion media comprises 1-50 ng/ml IL-2, 1-50 ng/ml IL-7, 1-100 ng/ml IL-12, 1-100 ng/ml IL-15, 1-100 ng/ml IL-18, and activation beads coated with anti-CD2/anti-NKp46 at a cell:bead ratio of 1:1. In some embodiments, the NK cell expansion media comprises a defined xenogenic-freebase media, comprises 1-50 ng/ml IL-2, 1-50 ng/ml IL-7, 1-100 ng/ml IL-12, 1-100 ng/ml IL-15, 1-100 ng/ml IL-18, and activation beads coated with anti-CD2/anti-NKp46 at a cell:bead ratio of 1:1.

### Differentiation Methods

In some aspects, the disclosure provides methods of generating hematopoietic progenitors from a stem cell in a 3D culture system. In some aspects, the disclosure provides methods of generating NK cells from a stem cell in a 3D culture system. In some aspects, the disclosure provides methods of generating NK cells from a hematopoietic progenitor in a 3D culture system. In some embodiments, a method of generating NK cells comprises differentiating a stem cell to a hematopoietic progenitor, and differentiating the hematopoietic progenitor to an NK cell in a 3D culture system.

In some aspects, the disclosure provides methods of generating a common lymphoid progenitor (CLP) from a stem cell in a 3D culture system. In some embodiments, the methods comprise differentiating a stem cell into a hematopoietic progenitor, and differentiating the hematopoietic progenitor into a CLP in a 3D culture system. CLPs refer to cells that are precursors to lymphoid cells. CLPs are cells capable of hematopoietic transition to hematopoietic cell-types. In some embodiments, CLPs are CD45+ CD7+ CD5+/lo CD3− CD56−. In some embodiments, CLPs are CD45+ CD5+/lo CD7+. In some aspects, the disclosure provides methods of generating NK cells from CLPs in a 3D culture system. In some embodiments a method of generating NK cells comprises differentiating a stem cell to a hematopoietic progenitor, differentiating the hematopoietic progenitor into a CLP, and differentiating the CLP into an NK cell in a 3D culture system.

In some aspects, the disclosure provides methods of generating a preNK cell progenitor (preNKP) from a stem cell in a 3D culture system. In some embodiments, the methods comprise differentiating a stem cell into a hematopoietic progenitor, differentiating the hematopoietic progenitor into a CLP, and differentiating the CLP into a PreNKP in a 3D culture system. PreNKPs are intermediate cells between CLPs and NKPs. In some embodiments, PreNKPs are Lin−/CD244+/c-Kit_{low}/IL-7Ra+/FLT3−/CD122− In some aspects, the disclosure provides methods of generating NK cells from preNKPs. In some embodiments a method of generating NK cells comprises differentiating a stem cell to a hematopoietic progenitor, differentiating the hematopoietic progenitor into a CLP, differentiating the CLP into a preNKP, and differentiating the preNKP into an NK cell in a 3D culture system.

In some aspects, the disclosure provides methods of generating a NK cell precursor (NKP) from a stem cell in a 3D culture system. In some embodiments, the methods comprise differentiating a stem cell into a hematopoietic progenitor, differentiating the hematopoietic progenitor into a CLP, differentiating the CLP into a PreNKP, and differentiating the PreNKP into an NKP in a 3D culture system. NKPs are the last cell before the final NK lineage commitment. In some embodiments, NKPs are Lin−/NK1.1-DX5−/IL-7Ra+/CD122+/NKG2D+. In some aspects, the disclosure provides methods of generating NK cells from NKPs in a 3D culture system. In some embodiments a method of generating NK cells comprises differentiating a stem cell to a hematopoietic progenitor, differentiating the hematopoietic progenitor into a CLP, differentiating the CLP into a preNKP, differentiating the preNKP into NKP, and differentiating the NKP into an NK cell in a 3D culture system.

In some aspects, the disclosure provides methods of generating an immature NK (iNK) cell from a stem cell in a 3D culture system. In some embodiments, the methods comprise differentiating a stem cell into a hematopoietic progenitor, differentiating the hematopoietic progenitor into a CLP, differentiating the CLP into a PreNKP, differentiating the PreNKP into an NKP, and differentiating the NKP into an iNK cell in a 3D culture system. In some aspects, the disclosure provides methods of generating NK cells from an iNK cell in a 3D culture system. In some embodiments a method of generating NK cells comprises differentiating a stem cell to a hematopoietic progenitor, differentiating the hematopoietic progenitor into a CLP, differentiating the CLP into a preNKP, differentiating the preNKP into an NKP, differentiating the NKP into an iNK cell, and differentiating the iNK cell into an NK cell in a 3D culture system.

In some aspects, the disclosure provides methods of generating a mature NK (mNK) cell from a stem cell in a 3D culture system. In some aspects, the disclosure provides methods of generating mature NK cells from an immature NK cell in a 3D culture system. In some embodiments a method of generating NK cells comprises differentiating a stem cell to a hematopoietic progenitor, differentiating the hematopoietic progenitor into a CLP, differentiating the CLP into a preNKP, differentiating the preNKP into a NKP, differentiating the NKP into an iNK cell, and differentiating the iNK cell into an mNK cell in a 3D culture system.

In some embodiments, the methods provided herein are xenogenic-free. In some embodiments, the methods provided herein are free of animal-derived raw materials.

### Expression Markers

Differentiation of source cells into NK cells can be assessed by detecting markers, e.g., CD56, CD94, CD117, NKG2D, DNAM-1 and NKp46, by, for example, flow cytometry. Differentiation can also be assessed by the morphological characteristics of NK cells, e.g., large size, high protein synthesis activity in the abundant endoplasmic reticulum (ER), and/or preformed granules. Maturation of NK cells can be assessed by detecting one or more functionally relevant makers, for example, CD94, CD161, NKp44, DNAM-1, 2B4, NKp46, CD94, KIR, and the NKG2 family of activating receptors (e.g., NKG2D). Maturation of NK cells can also be assessed by detecting specific markers during different developmental stages. For example, in one embodiment, preNKP cells are CD34+, CD45RA+, CD10+, CD117− and/or CD161−. In another embodiment, immature NK cells are CD34−, CD117+, CD161+, NKp46− and/or CD94/NKG2A−. In another embodiment, CD56bright NK cells are CD117+, NKp46+, CD94/NKG2A+, CD16−, and/or KIR+/−. In another embodiment, CD56dim NK cells are CD117−, NKp46+, CD94/NKG2A+/−, CD16+, and/or KIR+. In a specific embodiment, maturation of NK cells (e.g., TSNK cells) is determined by the percentage of NK cells (e.g., TSNK cells) that are CD161−, CD94+ and/or NKp46+. In a more specific embodiment, at least 10%, 20%, 25%, 30%, 35%, 40%, 50%, 55%, 60%, 65% or 70% of mature NK cells (e.g., TSNK cells) are NKp46+. In another more specific embodiments, at least 10%, 20%, 25%, 30%, 35%, 40%, 45% or 50% of mature NK cells (e.g., TSNK cells) are CD94+. In another more specific embodiments, at least 10%, 20%, 25%, 30%, 35%, 40%, 45% or 50% of mature NK cells (e.g., TSNK cells) are CD161−.

In some embodiments, the differentiation of source cells into NK cells are assessed by detecting the expression level of, e.g., CD3, CD7 or CD127, CD10, CD14, CD15, CD16, CD33, CD34, CD56, CD94, CD117, CD161, NKp44, NKp46, NKG2D, DNAM-1, 2B4 or TO-PRO-3, using, e.g., antibodies to one or more of these cell markers. Such antibodies can be conjugated to a detectable label, for example, as fluorescent label, e.g., FITC, R-PE, PerCP, PerCP-Cy5.5, APC, APC-Cy7 or APC-H7.

In some embodiments, the frequency of the cell population that has the desired expression pattern are higher in 3D culture compared to 2D culture. In some embodiments, the 3D culture system may produce at least 10%, 20%, 25%, 30%, 35%, 40%, 50%, 55%, 60%, 65% 70%, 75%, 80%, 85%, 90%, 95%, or 100% more hematopoietic progenitor cells (CD34+, CD45+, and CD43+) compared to 2D culture. In some embodiments, the 3D culture system may produce at least 10%, 20%, 25%, 30%, 35%, 40%, 50%, 55%, 60%, 65% 70%, 75%, 80%, 85%, 90%, 95%, or 100% more preNKP cells (CD34+, CD45RA+, CD10+, CD117− and/or CD161−) compared to 2D culture. In some embodiments, the 3D culture system produces at least 10%, 20%, 25%, 30%, 35%, 40%, 50%, 55%, 60%, 65% 70%, 75%, 80%, 85%, 90%, 95%, or 100% more immature NK cells (CD34−, CD117+, CD161+, NKp46− and/or CD94/NKG2A−) compared to 2D culture. In some embodiments, the 3D culture system produces at least 10%, 20%, 25%, 30%, 35%, 40%, 50%, 55%, 60%, 65% 70%, 75%, 80%, 85%, 90%, 95%, or 100% more CD56bright NK cells (CD117+, NKp46+, CD94/NKG2A+, CD16−, and/or KIR+/−) compared to 2D culture. In some embodiments, the 3D culture system produces at least 10%, 20%, 25%, 30%, 35%, 40%, 50%, 55%, 60%, 65% 70%, 75%, 80%, 85%, 90%, 95%, or 100% more CD56dim NK cells (CD117−, NKp46+, CD94/NKG2A+/−, CD16+, and/or KIR+) compared to 2D culture. In some embodiments, the 3D culture system produces at least 10%, 20%, 25%, 30%, 35%, 40%, 50%, 55%, 60%, 65% 70%, 75%, 80%, 85%, 90%, 95%, or 100% more mature NK cells (CD161−, CD94+ and/or NKp46+) compared to 2D culture.

### Source Cells

In some embodiments, NK cells are generated from source cells. Any progenitor cell known in the art may be used as a source cell in the methods of the disclosure.

In some embodiments, the source cells are hESCs. In some embodiments, the source cells are iPSCs. An NK cell derived from iPSCs may alternatively be referred to as iPSC-derived NK cells.

In immunotherapy, source cells be allogeneic or autologous, meaning from a donor or from the subject, respectively. In some embodiments, the source cells are allogeneic. In some embodiments, the source cells are autologous.

In some embodiments, source cells are peripheral blood cells. As used herein, the term “peripheral blood cell” is used to refer to cells that originate from circulating blood and comprise hematopoietic stem cells that are capable of proliferation, selectable differentiation, and maturation. As such, peripheral blood NK cells may alternatively be referred to as differentiated blood-derived NK cells (bdNK).

In some embodiments, source cells include hematopoietic stem cells, characterized as being CD34+ and/or CD45+.

In some embodiments, source cells include common lymphoid progenitor cells, characterized as being CD45+ CD7+ CD56−.

In some embodiments, NK cells may be generated from induced pluripotent stem cells (iPSCs). iPSCs are a type of pluripotent stem cell derived from adult somatic cells that have been genetically reprogrammed to an embryonic stem cell-like state through the forced expression of genes and factors important for maintaining the defining properties of embryonic stem cells. iPSCs may be generated from tissues with somatic cells, including, but not limited to, the skin, dental tissue, peripheral blood, and urine. To generate iPSCs, somatic cells may be reprogrammed through methods including, but not limited to, the transient expression of reprogramming factors, virus-free methods, adenoviruses, plasmids, minicircle vectors, episomal vectors, Sendai viruses, synthetic mRNAs, self-replicating RNAs, retroviruses, lentiviruses, PhiC31 integrases, excisable transposons, CRISPR-based gene editing, or recombinant proteins. Methods for generating iPSCs are disclosed in U.S. Pat. No. 9,315,779 B2, U.S. Ser. No. 10/370,452 B2, U.S. Ser. No. 11/319,555 B2, and US20210015859A1, which are incorporated by reference in their entirety.

### Mesoderm/Embryoid Body Formation

In some embodiments, the methods described herein comprise generating mesoderm cells from iPSCs and/or hESCs. As stem cells begin to differentiate, three distinct germ layers are formed: the ectoderm, mesoderm, and endoderm. Immune cells, such as NK cells, differentiate from mesoderm cells. In some embodiments, the mesoderm cells produced by the methods of the disclosure are further differentiated to NK cells.

The mesoderm formation step may comprise contacting the iPSC or hESC cell population with one or more factors, in a defined expansion media, for specified period of time. In some embodiments, mesoderm cells are formed from embryoid bodies.

In some embodiments, stem cells are contacted with a differentiation media described herein for a period of time to generate mesoderm cells and/or embryoid bodies. In some embodiments, the iPSC or hESC cell population is contacted with the differentiation media in a 3D culture system for a period of time sufficient to generate mesoderm cells and/or embryoid bodies. In some embodiments, the period of time sufficient to generate mesoderm cells from stem cells is at least 24 hours, at least 48 hours, at least 72 hours, at least 96 hours, or at least 120 hours.

In some embodiments, the mesoderm formation step has duration of about 12 hours to 24 hours, about 24 hours to 48 hours, about 48 hours to 72 hours, about 72 hours to 96 hours, or about 96 hours to 120 hours.

In some embodiments, a cell derived from a source cell is disposed in a vessel to induce the cells to aggregate and form clusters. In some embodiments, the vessel is a plate with wells or microwells, such as a 96-well plate and/or an Aggrewell™ plate (microwell plate; STEMCELL Technologies Inc., Vancouver, Canada). In some embodiments, using, for example, an Aggrewell™ plate, cell clusters are prepared in a plate having microwells to form aggregates of cells of uniform size and shape. In some embodiments, at least 1 cell, at least 10 cells, at least 100 cells, at least 1,000 cells, at least 10,000 cells, or at least 50,000 cells are seeded in each well. In some embodiments, about 1 cell to 10 cells, about 10 cells to 100 cells, about 100 cells to 1,000 cells, about 1,000 cells to 10,000 cells, or about 10,000 cells to 50,000 cells are seeded in each well.

Differentiation into Hematopoietic Progenitors

In some aspects, the disclosure provides methods of generating NK cells from a hematopoietic progenitor cells. In some embodiments, a method of generating NK cells comprises differentiating the hematopoietic progenitor to an NK cell. In some embodiments, the methods provided herein are xenogenic-free.

An aspect of the disclosure is that the method of producing NK cells may include a hematopoietic progenitor differentiation step. The hematopoietic progenitor differentiation step may comprise contacting the embryoid body cell population with one or more factors, in a defined differentiation media, for specified period of time, thereby inducing formation of hematopoietic progenitors in the cell population. The hematopoietic progenitors are then defined by expressing a combination of markers.

In some embodiments, mesoderm and/or embryoid body cells are contacted with a differentiation media described herein for a period of time to generate hematopoietic progenitor cells. In some embodiments, the mesoderm and/or embryoid body cells are contacted with the differentiation media in a 3D culture system for a period of time sufficient to generate hematopoietic progenitor cells. In some embodiments, the period of time sufficient to generate hematopoietic progenitor cells from mesoderm and/or embryoid body cells is at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, or at least 20 days.

In some embodiments, the differentiation into hematopoietic progenitors step has duration of about 1 day to 2 days, about 2 days to 3 days, about 3 days to 4 days, about 4 days to 5 days, about 5 days to 6 days, about 6 days to 7 days, about 7 days to 8 days, about 8 days to 9 days, about 9 days to 10 days, about 10 days to 11 days, about 11 days to 12 days, about 12 days to 13 days, about 13 days to 14 days, about 14 days to 15 days, about 15 days to 16 days, about 16 days to 17 days, about 17 days to 18 days, about 18 days to 19 days, or about 19 days to 20 days.

In some embodiments, the hematopoietic progenitor cells express CD34, CD43 and CD45. In some embodiments, the method of the disclosure increases the percentage of CD34+CD43+CD45+ triple-positive cells.

In some embodiments, the methods of the disclosure generate a population of cells from the iPSCs with a purity of at least 40%, at least 50%, at least 60%, at least 70%, at least 80%, at least 90% or 100% of CD34+CD43+CD45+ triple-positive cells.

In some embodiments, the methods of the disclosure generate a population of cells from the iPSCs with a purity of about 40% to 50%, about 50% to 60%, about 60% to 70%, about 70% to 80%, about 80% to 90%, or about 90% to 100% of CD34+CD43+CD45+ triple-positive cells.

Differentiation into NK Cells

In some aspects, the disclosure provides methods of generating NK cells from hematopoietic progenitor cells. In some embodiments, a method of generating NK cells comprises differentiating the hematopoietic progenitor cells. In some embodiments, the methods provided herein are xenogenic-free.

An aspect of the disclosure is that the method of producing NK cells may include a NK differentiation step. The NK differentiation step may comprise contacting the HP cell population with one or more factors, in a defined differentiation media, for specified period of time, thereby inducing formation of NK cells in the cell population. In some embodiments, the HP cell population is contacted with a differentiation media in a 3D suspension culture for a period of time sufficient to form NK cells. The NK cells are then defined by expressing a combination of markers.

In some embodiments, hematopoietic progenitor cells are contacted with a differentiation media described herein for a period of time to generate NK cells. In some embodiments, the period of time sufficient to generate NK cells from hematopoietic progenitor cells is at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, or at least 40 days,

In some embodiments, the NK differentiation step has duration of about 1 day to 2 days, about 2 days to 3 days, about 3 days to 4 days, about 4 days to 5 days, about 5 days to 6 days, about 6 days to 7 days, about 7 days to 8 days, about 8 days to 9 days, about 9 days to 10 days, about 10 days to 11 days, about 11 days to 12 days, about 12 days to 13 days, about 13 days to 14 days, about 14 days to 15 days, about 15 days to 16 days, about 16 days to 17 days, about 17 days to 18 days, about 18 days to 19 days, or about 19 days to 20 days, about 20 days to 21 days, about 21 days to 22 days, about 22 days to 23 days, about 23 days to 24 days, about 24 days to 25 days, about 25 days to 26 days, about 26 days to 27 days, about 27 days to 28 days, about 28 days to 29 days, about 29 days to 30 days, about 30 days to 31 days, about 31 days to 32 days, about 32 days to 33 days, about 33 days to 34 days, about 34 days to 35 days, about 35 days to 36 days, about 36 days to 37 days, about 37 days to 38 days, about 38 days to 39 days, or about 39 days to 40 days.

In some embodiments, the differentiated NK cells comprise the markers CD34, CD43, CD45, and LFA1. In some embodiments, the method of the disclosure increases the percentage of CD34+CD43+CD45+LFA1+ quadruple-positive cells.

In some embodiments, the methods of the disclosure generate a population of NK cells from the hematopoietic progenitor cells with a purity of at least 40%, at least 50%, at least 60%, at least 70%, at least 80%, at least 90% or 100% of CD34+CD43+CD45+LFA1+ quadruple-positive cells.

In some embodiments, the methods of the disclosure generate a population of NK cells from the hematopoietic progenitor cells with a purity of about 40% to 50%, about 50% to 60%, about 60% to 70%, about 70% to 80%, about 80% to 90%, or about 90% to 100% of CD34+CD43+CD45+LFA1+ quadruple-positive cells.

### NK Cell Maturation

In some aspects, the disclosure provides methods of generating mature NK cells from differentiated NK cells. In some embodiments, a method of generating mature NK cells comprises differentiating the NK cells. In some embodiments, the methods provided herein are xenogenic-free.

An aspect of the disclosure is that the method of producing NK cells may include a NK maturation step. The NK maturation step may comprise contacting the differentiated NK cell population with one or more factors, in a defined expansion media, for specified period of time, thereby inducing NK cell maturation in the cell population. In some embodiments, the method comprises contacting the differentiated NK cell population in a 3D culture system with one or more factors for a period of time sufficient to induce NK cell maturation. The mature NK cells are then defined by expressing a combination of markers.

In some embodiments, differentiated NK cells are contacted with a maturation media described herein for a period of time to generate mature NK cells. In some embodiments, the period of time sufficient to generate mature NK cells from hematopoietic progenitor cells is at least 24 hours, at least 48 hours, at least 72 hours, at least 96 hours, at least 120 hours at least 144 hours, at least 168 hours, at least 192 hours, at least 216 hours, or at least 240 hours.

In some embodiments, the maturation step has duration of about 12 hours to 24 hours, about 24 hours to 48 hours, about 48 hours to 72 hours, about 72 hours to 96 hours, about 96 hours to 120 hours, about 120 hours to 144 hours, about 144 hours to 168 hours, about 168 hours to 192 hours, about 192 hours to 216 hours, or about 216 hours to 240 hours.

In some embodiments, the mature NK cells comprise the markers CD34, CD43, CD45, and LFA1. In some embodiments, the method of the disclosure increases the percentage of CD34+CD43+CD45+LFA1+ quadruple-positive cells. In some embodiments, the method increases expression of activation markers. In some embodiments, the activation markers comprise NKp46, NKG2D, LFA1, and/or CD16. In some embodiments, the method decrease expression of inhibitory markers. In some embodiments, the inhibitory markers comprise CD161 and CD73.

In some embodiments, the methods of the disclosure generate a population of mature NK cells from the differentiated NK cells with a purity of at least 40%, at least 50%, at least 60%, at least 70%, at least 80%, at least 90% or 100% of CD34+CD43+CD45+LFA1+ NKp46+NKG2D+LFA1+CD161−CD73− cells.

In some embodiments, the methods of the disclosure generate a population of mature NK cells from the differentiated NK cells with a purity of about 40% to 50%, about 50% to 60%, about 60% to 70%, about 70% to 80%, about 80% to 90%, or about 90% to 100% of CD34+CD43+CD45+LFA1+ NKp46+NKG2D+LFA1+CD161−CD73− cells.

In some embodiments, the maturation step decreases the population of CD56− cells.

In some embodiments, the methods of the disclosure generate a population of mature NK cells from the differentiated NK cells with a purity of at least 40%, at least 50%, at least 60%, at least 70%, at least 80%, at least 90% or 100% of CD56− cells.

In some embodiments, the methods of the disclosure generate a population of mature NK cells from the differentiated NK cells with a purity of about 40% to 50%, about 50% to 60%, about 60% to 70%, about 70% to 80%, about 80% to 90%, or about 90% to 100% of CD56− cells.

### Exemplary Differentiation Methods

In some embodiments, a method of differentiating stem cells into hematopoietic progenitors comprises contacting a population of stem cells with a xenogenic-free differentiation media comprising BMP4, FGF2, and VEGF in a 3D culture system. In some embodiments, a method of differentiating stem cells into hematopoietic progenitors comprises contacting a population of stem cells with a xenogenic-free differentiation media comprising 1-50 ng/mL BMP4, 1-150 ng/mL FGF2, and 5-100 ng/mL VEGF in a 3D culture system. In some embodiments, a method of differentiating stem cells into hematopoietic progenitors comprises contacting a population of stem cells with a xenogenic-free differentiation media comprising BMP4, FGF2, and VEGF, for 12 to 120 hours in a 3D culture system. In some embodiments, a method of differentiating stem cells into hematopoietic progenitors comprises contacting a population of stem cells with a xenogenic-free differentiation media comprising 1-50 ng/mL BMP4, 1-150 ng/mL FGF2, and 5-100 ng/mL VEGF, for 12 to 120 hours in a 3D culture system.

In some embodiments, a method of differentiating stem cells into hematopoietic progenitors comprises contacting a population of stem cells with a xenogenic-free differentiation media comprising BMP4, FGF2, VEGF, and a ROCK inhibitor in a 3D culture system. In some embodiments, a method of differentiating stem cells into hematopoietic progenitors comprises contacting a population of stem cells with a xenogenic-free differentiation media comprising 1-50 ng/mL BMP4, 1-150 ng/mL FGF2, 5-100 ng/mL VEGF, and 0.1-20 μM of a ROCK inhibitor in a 3D culture system. In some embodiments, a method of differentiating stem cells into hematopoietic progenitors comprises contacting a population of stem cells with a xenogenic-free differentiation media comprising BMP4, FGF2, VEGF, and a ROCK inhibitor, for 12-120 hours in a 3D culture system. In some embodiments, a method of differentiating stem cells into hematopoietic progenitors comprises contacting a population of stem cells with a xenogenic-free differentiation media comprising 1-50 ng/mL BMP4, 1-150 ng/mL FGF2, 5-100 ng/mL VEGF, and 0.1-20 μM of a ROCK inhibitor, for 12 to 120 hours in a 3D culture system.

In some embodiments, a method of differentiating stem cells into hematopoietic progenitors comprises contacting a population of stem cells with a serum-free differentiation media comprising BMP4, FGF2, VEGF, and Y27632 in a 3D culture. In some embodiments, a method of differentiating stem cells into hematopoietic progenitors comprises contacting a population of stem cells with a xenogenic-free differentiation media comprising 1-50 ng/mL BMP4, 1-150 ng/mL FGF2, 5-100 ng/mL VEGF, and 0.1-20 μM Y27632 in a 3D culture system. In some embodiments, a method of differentiating stem cells into hematopoietic progenitors comprises contacting a population of stem cells with a xenogenic-free differentiation media comprising BMP4, FGF2, VEGF, and Y27632, for 12-120 hours in a 3D culture system. In some embodiments, a method of differentiating stem cells into hematopoietic progenitors comprises contacting a population of stem cells with a xenogenic-free differentiation media comprising 1-50 ng/mL BMP4, 1-150 ng/mL FGF2, 5-100 ng/mL VEGF, and 0.1-20 μM Y27632, for 12 to 120 hours in a 3D culture system.

In some embodiments, a method of differentiating stem cells into hematopoietic progenitors comprises (i) contacting a population of stem cells with a first xenogenic-free differentiation media comprising BMP4, FGF2, and VEGF in a 3D culture system to generate a population of mesoderm cells, and (ii) contacting the population of mesoderm cells with a second xenogenic-free differentiation media comprising BMP4, FGF2, VEGF, SCF, LDL, and TPO and in a 3D culture system to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-150 ng/mL FGF2, and 5-100 ng/mL VEGF in a 3D culture system to generate a population of mesoderm cells, and (ii) contacting the population of mesoderm cells with a second xenogenic-free differentiation media comprising 1-50 ng/mL BMP4, 1-150 ng/mL FGF2, 5-100 ng/mL VEGF, about 1-100 ng/mL SCF, about 1-50 ug/mL LDL, and about 1-100 ng/mL TPO in a 3D culture system to generate hematopoietic progenitors. In some embodiments, a method of differentiating stem cells into hematopoietic progenitors comprises (i) contacting a population of stem cells with a first xenogenic-free differentiation media comprising BMP4, FGF2, and VEGF for 12-120 hours in a 3D culture system to generate a population of mesoderm cells, and (ii) contacting the population of mesoderm cells with a second xenogenic-free differentiation media comprising BMP4, FGF2, VEGF, SCF, LDL, and TPO for 2-20 days in a 3D culture to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-150 ng/mL FGF2, and 5-100 ng/mL VEGF for 12-120 hours in a 3D culture system to generate a population of mesoderm cells, and (ii) contacting the population of mesoderm cells with a second xenogenic-free differentiation media comprising 1-50 ng/mL BMP4, 1-150 ng/mL FGF2, 5-100 ng/mL VEGF, about 1-100 ng/mL SCF, about 1-50 ug/mL LDL, and about 1-100 ng/mL TPO for 2-20 days in a 3D culture system to generate hematopoietic progenitors.

In some embodiments, a method of differentiating stem cells into hematopoietic progenitors comprises (i) contacting a population of stem cells with a first xenogenic-free differentiation media comprising BMP4, FGF2, VEGF, and a ROCK inhibitor in a 3D culture system to generate a population of mesoderm cells, and (ii) contacting the population of mesoderm cells with a second xenogenic-free differentiation media comprising BMP4, FGF2, VEGF, SCF, LDL, and TPO in a 3D culture system to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-150 ng/mL FGF2, 5-100 ng/mL VEGF, and 0.1-20 μM of a ROCK inhibitor in a 3D culture system to generate a population of mesoderm cells, and (ii) contacting the population of mesoderm cells with a second xenogenic-free differentiation media comprising 1-50 ng/mL BMP4, 1-150 ng/mL FGF2, 5-100 ng/mL VEGF, about 1-100 ng/mL SCF, about 1-50 ug/mL LDL, and about 1-100 ng/mL TPO in a 3D culture system to generate hematopoietic progenitors. In some embodiments, a method of differentiating stem cells into hematopoietic progenitors comprises (i) contacting a population of stem cells with a first xenogenic-free differentiation media comprising BMP4, FGF2, VEGF and a ROCK inhibitor for 12-120 hours in a 3D culture system to generate a population of mesoderm cells, and (ii) contacting the population of mesoderm cells with a second xenogenic-free differentiation media comprising BMP4, FGF2, VEGF, SCF, LDL, and TPO for 2-20 days in a 3D culture system to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-150 ng/mL FGF2, 5-100 ng/mL VEGF, and 0.1-20 μM of a ROCK inhibitor for 12-120 hours in a 3D culture system to generate a population of mesoderm cells, and (ii) contacting the population of mesoderm cells with a second xenogenic-free differentiation media comprising 5-50 ng/mL BMP4, 1-150 ng/mL FGF2, 5-100 ng/mL VEGF, about 1-100 ng/mL SCF, about 1-50 ug/mL LDL, and about 1-100 ng/mL TPO for 2-20 days in a 3D culture system to generate hematopoietic progenitors.

In some embodiments, a method of differentiating stem cells into hematopoietic progenitors comprises (i) contacting a population of stem cells with a first xenogenic-free differentiation media comprising BMP4, FGF2, VEGF, and Y27632 in a 3D culture system to generate a population of mesoderm cells, and (ii) contacting the population of mesoderm cells with a second xenogenic-free differentiation media comprising BMP4, FGF2, VEGF, SCF, LDL, and TPO in a 3D culture system to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-150 ng/mL FGF2, 5-100 ng/mL VEGF, and 0.1-20 μM Y27632 in a 3D culture system to generate a population of mesoderm cells, and (ii) contacting the population of mesoderm cells with a second xenogenic-free differentiation media comprising 1-50 ng/mL BMP4, 1-150 ng/mL FGF2, 5-100 ng/mL VEGF, about 1-100 ng/mL SCF, about 1-50 ug/mL LDL, and about 1-100 ng/mL TPO in a 3D culture system to generate hematopoietic progenitors. In some embodiments, a method of differentiating stem cells into hematopoietic progenitors comprises (i) contacting a population of stem cells with a first xenogenic-free differentiation media comprising BMP4, FGF2, VEGF and Y27632 for 12-120 hours in a 3D culture system to generate a population of mesoderm cells, and (ii) contacting the population of mesoderm cells with a second xenogenic-free differentiation media comprising BMP4, FGF2, VEGF, SCF, LDL, and TPO for 2-20 days in a 3D culture system to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-150 ng/mL FGF2, 5-100 ng/mL VEGF, and 0.1-20 μM Y27632 for 12-120 hours in a 3D culture system to generate a population of mesoderm cells, and (ii) contacting the population of mesoderm cells with a second xenogenic-free differentiation media comprising 1-50 ng/mL BMP4, 1-150 ng/mL FGF2, 5-100 ng/mL VEGF, about 1-100 ng/mL SCF, about 1-50 ug/mL LDL, and about 1-100 ng/mL TPO 2-20 days in a 3D culture system to generate hematopoietic progenitors.

In some embodiments, the hematopoietic progenitor cell formation step is followed by an NK cell differentiation step. In some embodiments, the method of differentiating HPs into NK comprises contacting a population of HP cells with a xenogenic-free NK differentiation media in a 3D culture system. In some embodiments, the method of differentiating HPs into NK comprises contacting a population of HP cells with a xenogenic-free NK differentiation media for 15-25 days in a 3D culture system.

In some embodiments, a method of differentiating stem cells into NK cells comprises (i) contacting a population of stem cells with a first xenogenic-free differentiation media comprising BMP4, FGF2, and VEGF to generate a population of mesoderm cells, (ii) contacting the population of mesoderm cells with a second xenogenic-free differentiation media comprising BMP4, FGF2, VEGF, SCF, LDL, TPO, and a PI3K inhibitor to generate hematopoietic progenitors, and (iii) contacting the population of hematopoietic progenitor cells with a third xenogenic-free NK differentiation media to generate NK cells in a 3D culture system.

In some embodiments, the method of differentiating stem cells into NK cells comprises (i) contacting a population of stem cells with a first xenogenic free differentiate media comprising 5-50 ng/mL BMP4, 5-50 ng/mL FGF2, and 5-100 ng/mL VEGF to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of a PI3K inhibitor to generate hematopoietic progenitors, and (iii) contacting the population of hematopoietic progenitor cells with a third xenogenic-free NK differentiation media in a 3D culture system to generate NK cells.

In some embodiments, a method of differentiating stem cells into hematopoietic progenitors comprises (i) contacting a population of stem cells with a first xenogenic-free differentiation media comprising BMP4, FGF2, and VEGF for 12-120 hours in a 3D culture system to generate a population of mesoderm cells, (ii) contacting the population of mesoderm cells with a second xenogenic-free differentiation media comprising BMP4, FGF2, VEGF, SCF, LDL, TPO, and a PI3K inhibitor for 2-20 days in a 3D culture system that to generate hematopoietic progenitors, and (iii) contacting the population of hematopoietic progenitor cells with a third xenogenic-free NK differentiation media for 15-25 days in a 3D culture system to generate NK cells.

In some embodiments, a method of differentiating stem cells into NK cells comprises (i) contacting a population of stem cells with a first xenogenic-free differentiation media comprising BMP4, FGF2, VEGF, and a ROCK inhibitor in a 3D culture system to generate a population of mesoderm cells, (ii) contacting the population of mesoderm cells with a second xenogenic-free differentiation media comprising BMP4, FGF2, VEGF, SCF, LDL, and TPO, in a 3D culture system to generate hematopoietic progenitors, and (iii) contacting the population of hematopoietic progenitor cells with a third xenogenic-free NK differentiation media in a 3D culture system to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of a ROCK inhibitor in a 3D culture system to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, and about 100 ng/mL TPO, in a 3D culture system to generate hematopoietic progenitors, and (iii) contacting the population of hematopoietic progenitor cells with a third xenogenic-free NK differentiation media in a 3D culture system to generate NK cells.

In some embodiments, a method of differentiating stem cells into hematopoietic progenitors comprises (i) contacting a population of stem cells with a first xenogenic-free differentiation media comprising BMP4, FGF2, VEGF, a ROCK inhibitor for 12-120 hours in a 3D culture system to generate a population of mesoderm cells, (ii) contacting the population of mesoderm cells with a second xenogenic-free differentiation media comprising BMP4, FGF2, VEGF, SCF, LDL, and TPO for 2-20 days in a 3D culture system to generate hematopoietic progenitors, and (iii) contacting the population of hematopoietic progenitor cells with a third xenogenic-free NK differentiation media in a 3D culture system for 15-25 days to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of a ROCK inhibitor in a 3D culture system for 12-120 hours to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, and about 100 ng/mL TPO, in a 3D culture system for 2-20 days to generate hematopoietic progenitors, and (iii) contacting the population of hematopoietic progenitor cells with a third xenogenic-free NK differentiation media in a 3D culture system for 15-25 days to generate NK cells.

In some embodiments, a method of differentiating stem cells into NK cells comprises (i) contacting a population of stem cells with a first xenogenic-free differentiation media comprising BMP4, FGF2, VEGF, and Y27632 in a 3D culture system to generate a population of mesoderm cells, (ii) contacting the population of mesoderm cells with a second xenogenic-free differentiation media comprising BMP4, FGF2, VEGF, SCF, LDL, and TPO, a in a 3D culture system to generate hematopoietic progenitors, and (iii) contacting the population of hematopoietic progenitor cells with a third xenogenic-free NK differentiation media in a 3D culture system to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of Y27632 in a 3D culture system to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, and about 100 ng/mL TPO, in a 3D culture system to generate hematopoietic progenitors, and (iii) contacting the population of hematopoietic progenitor cells with a third xenogenic-free NK differentiation media in a 3D culture system to generate NK cells.

In some embodiments, a method of differentiating stem cells into hematopoietic progenitors comprises (i) contacting a population of stem cells with a first xenogenic-free differentiation media comprising BMP4, FGF2, VEGF, and Y27632 in a 3D culture system for 12-120 hours to generate a population of mesoderm cells, (ii) contacting the population of mesoderm cells with a second xenogenic-free differentiation media comprising BMP4, FGF2, VEGF, SCF, LDL, and TPO, in a 3D culture system for 2-20 days to generate hematopoietic progenitors, and (iii) contacting the population of hematopoietic progenitor cells with a third xenogenic-free NK differentiation media in a 3D culture system for 15-25 days to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of Y27632 in a 3D culture system for 12-120 hours to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, and about 100 ng/mL TPO, in a 3D culture system for 2-20 days to generate hematopoietic progenitors, and (iii) contacting the population of hematopoietic progenitor cells with a third xenogenic-free NK differentiation media in a 3D culture system for 15-25 days to generate NK cells.

In some or any of the foregoing embodiments, the NK differentiation media comprises SCF, IL-7, IL-15, IL-12, FLT3L, a pyrimido-indole derivative, and an aryl hydrocarbon receptor antagonist. In some embodiments, the pyrimido-indole derivative is UM729. In some embodiments, the aryl hydrocarbon receptor is SR1. In some embodiments, the NK differentiation media comprises SCF, IL-7, IL-15, IL-12, FLT3L, UM729, and SR1.

In some or any of the foregoing embodiments, the NK differentiation media comprises 5-50 ng/mL SCF, 5-50 ng/mL IL-7, 5-100 ng/mL IL-15, 5-100 ng/mL IL-12, 5-100 ng/mL FLT3L, 1-10 μM of a pyrimido-indole derivative, and 1-10 μM of an aryl hydrocarbon receptor antagonist. In some embodiments, the NK differentiation media comprises 5-50 ng/mL SCF, 5-50 ng/mL IL-7, 5-100 ng/mL IL-15, 5-100 ng/mL 11-12, 5-100 ng/mL FLT3L, 1-10 μM of UM729, and 1-10 μM of SR1.

In some or any of the foregoing embodiments, the 3D culture system is agitated at about 50 RPM to about 100 RPM. In some or any of the foregoing embodiments, the 3D culture system is a bioreactor.

In some embodiments, about the stem cells and cells differentiated therefrom comprise a genetic edit. In some embodiments, the genetic edit is a gene knockout. In some embodiments, the genetic edit is an FKBP12 gene knockout. In some embodiments, the genetic edit is a B2M gene knockout.

In some embodiments, the stem cells comprise a knock-in gene. In some embodiments, the knock-in gene encodes an exogenous receptor. In some embodiments, the exogenous receptor is a chimeric antigen receptor (CAR). In some embodiments, the exogenous receptor is a rapamycin-activated cytokine receptor (RACR). In some embodiments, the stem cells and cells differentiated therefrom comprise a gene knockout and a gene knock-in. In some embodiments, the gene knock-in is located in the gene knockout.

### Characteristics of NK Cells

In some embodiments, the NK cells produced by the 3D suspension culture methods of the disclosure have improved or enhanced properties compared to NK cells produced by 2D culture methods.

In some embodiments, the NK cells produced by the 3D suspension culture methods of the disclosure have enhanced expansion. In some embodiments, the NK cells have at least a 50 fold expansion, at least a 100 fold expansion, at least a 150 fold expansion, at least a 200 fold expansion, at least a 250 fold expansion, at least a 300 fold expansion, at least a 350 fold expansion, at least a 400 fold expansion, at least a 450 fold expansion, at least a 500 fold expansion, at least a 1,000 fold expansion, at least a 10,000 fold expansion, at least a 100,000 fold expansion, or at least a 1,000,000 fold expansion compared to NK cells produced by a 2D culture methods.

In some embodiments, the NK cells have about a 50 fold expansion to a 100 fold expansion, about a 100 fold expansion to a 150 fold expansion, about a 150 fold expansion to a 200 fold expansion, about a 200 fold expansion to a 250 fold expansion, about a 250 fold expansion to a 300 fold expansion, about a 300 fold expansion to a 350 fold expansion, about a 350 fold expansion to a 400 fold expansion, about a 400 fold expansion to a 450 fold expansion, about a 450 fold expansion to a 500 fold expansion, about a 500 fold expansion to a 1,000 fold expansion, about a 1,000 fold expansion to a 10,000 fold expansion, about a 10,000 fold expansion to a 100,000 fold expansion, or about a 100,000 fold expansion to a 1,000,000 fold expansion compared to NK cells produced by other 2D culture methods.

In some embodiments, the differentiated NK cells produced by the 3D suspension culture methods of the disclosure reduce more tumor cell growth compared to differentiated NK cells produced by 2D culture methods. In some embodiments, the differentiated NK cells produced by the 3D suspension culture methods reduce a tumor cell growth at least 50%, at least 60%, at least 70%, at least 80%, at least 90%, or 100% more than differentiated NK cells produced by 2D culture methods.

In some embodiments, the differentiated NK cells produced by the 3D suspension culture methods reduce a tumor cell growth about 50% to 60%, about 60% to 70%, about 70% to 80%, about 80% to 90%, or about 90% to 100% more than differentiated NK cells produced by 2D culture methods.

In some embodiments, the mature NK cells produced by the 3D suspension culture methods of the disclosure reduce more tumor cell growth compared to a 2D culture methods. In some embodiments, the mature NK cells produced by the 3D suspension culture methods reduce tumor cell growth at least 50%, at least 60%, at least 70%, at least 80%, at least 90%, or 100% more than mature NK cells produced by 2D culture methods.

In some embodiments, the mature NK cells produced by the 3D suspension culture methods reduce tumor cell growth about 50% to 60%, about 60% to 70%, about 70% to 80%, about 80% to 90%, or about 90% to 100% more than mature NK cells produced by 2D culture methods.

In some embodiments, the methods of the disclosure produce a differentiated NK cell population. In some embodiments, the 3D suspension culture methods of the disclosure produce a differentiated NK cell population that is at least 40%, at least 50%, at least 60%, at least 70%, at least 80%, at least 900 or 100% more CD34+CD43+CD45+LFA1+ quadruple-positive cells compared to 2D culture methods.

In some embodiments, the 3D suspension culture methods of the disclosure produce a differentiated NK cell population that is about 40% to 50%, about 50% to 60%, about 60% to 70%, about 70% to 80%, about 80% to 90%, or about 90% to 100% more CD34+CD43+CD45+LFA1+ quadruple-positive cells compared to 2D culture methods.

In some embodiments, the 3D suspension culture methods of the disclosure produce a mature NK cell population. In some embodiments, the methods of the disclosure produce a mature NK cell population that is at least 40%, at least 50%, at least 60%, at least 70%, at least 80%, at least 90% or 100% more CD34+CD43+CD45+LFA1+ NKp46+NKG2D+LFA1+CD161−CD73− cells compared to 2D culture methods.

In some embodiments, the 3D suspension culture methods of the disclosure produce a mature NK cell population that is about 40% to 50%, about 50% to 60%, about 60% to 70%, about 70% to 80%, about 80% to 90%, or about 90% to 100% more CD34+CD43+CD45+LFA1+ NKp46+NKG2D+LFA1+CD161−CD73− cells compared to 2D culture methods.

### Gene Editing

Genome editing generally refers to the process of editing or changing the nucleotide sequence of a genome, preferably in a precise, desirable and/or pre-determined manner. Examples of compositions, systems, and methods of genome editing described herein use site-directed nucleases to cut or cleave DNA at precise target locations in the genome, thereby creating a double-strand break (DSB) in the DNA. Such breaks can be repaired by endogenous DNA repair pathways, such as homology directed repair (HDR) and/or non-homologous end-joining (NHEJ) repair (see e.g., Cox et al., (2015) Nature Medicine 21 (2):121-31).

In some embodiments, the cells described herein (e.g., stem cells, HPs, NKs) are genetically modified. In some embodiments, the modification involves knocking out one or more endogenous genes using a DNA-targeted protein and a nuclease or an RNA-guided nuclease and/or knocking in exogenous genes of interest. In some embodiments, a gene of interest is knocked into a particular locus of interest. In some embodiments, the gene of interest is a synthetic cytokine receptor complex. In some embodiments, the gene of interest is a chimeric antigen receptor (CAR). In some embodiments, a RACR is knocked into a locus of interest.

In some embodiments, the modification comprises contacting a cell with a DNA-targeted protein and a nuclease or an RNA-guided nuclease. In some embodiments, DNA-targeted protein and a nuclease or an RNA-guided nuclease includes zinc finger protein (ZFP), a clustered regularly interspaced short palindromic nucleic acid (CRISPR), or a TAL-effector nuclease (TALEN). In some embodiments, Crispr-CAS9 is used. In some embodiments, Crispr-MAD7 is used.

Rejection of cellular therapeutics (e.g., CAR T cells) is due at least to mismatches of human leukocyte antigen (HLA) between donor and recipient. One solution recently identified is disrupting expression of genes involved in this rejection, such as T cell receptor alpha constant (TRAC), beta-2-microglobulin (B2M), and signal regulatory protein alpha (SIRPA). Accordingly, in some embodiments, the cells described herein (e.g., iPSCs, CILs), are genetically engineered to knockout a B2M locus, a TRAC locus, and/or a SIRPA locus. In some embodiments, the cells described herein are genetically engineered to knockout a B2M locus. In some embodiments, the cells described herein are genetically engineered to knockout a TRAC locus. In some embodiments, the cells described herein are genetically engineered to knockout a SIPRA locus.

In some embodiments, the cells described herein are genetically engineered to be rapamycin resistant. In some embodiments, the cells are genetically engineered to disrupt a gene associated with rapamycin recognition. In some embodiments, the cells are genetically engineered to disrupt the mTOR gene. In some embodiments, the cells are genetically engineered to disrupt the FKBP12 gene. In some embodiments, the cells are genetically engineered to knockout the FKB12 gene to induce rapamycin resistance.

In some embodiments, the cells described herein are genetically engineered to comprise a nucleotide sequence encoding a synthetic cytokine receptor in an endogenous gene. In some embodiments, the synthetic cytokine receptor is engineered into a gene such that expression of the endogenous gene is not disrupted. In some embodiments, the synthetic cytokine receptor is engineered into a safe-harbor locus.

In some embodiments, the cells described herein genetically engineered to comprise a nucleotide sequence encoding a synthetic cytokine receptor in a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB).

In some embodiments, the gene of interest inserted into an endogenous locus is a synthetic cytokine receptor complex. In some embodiments, the endogenous promoter of the particular locus is used. In some embodiments, additional promoter(s) may be included such that two or more promoters drive expression of the exogenous gene of interest.

In some embodiments, the cells described herein are genetically engineered to comprise a nucleotide sequence encoding a synthetic cytokine receptor complex in a disrupted gene. For example, in some embodiments the cells comprise a disrupted B2M gene and a nucleotide sequence encoding the synthetic cytokine receptor in the disrupted B2M gene.

In some embodiments, the cells described herein (e.g., iPSCs, CILs) comprise (i) a disrupted B2M locus, and (ii) a nucleotide sequence encoding a synthetic cytokine receptor complex under control of the endogenous B2M promoter and an EEF1A promoter.

In some embodiments, the cells described herein (e.g., iPSCs, HPs, NKs) comprise (i) a disrupted B2M locus, and (ii) a nucleotide sequence encoding a synthetic cytokine receptor complex inserted into the endogenous B2M gene and under control of the endogenous B2M promoter and an EEF1A promoter.

### Systems for Genome Editing

In some embodiments, a system for editing a cell described herein comprises a site-directed nuclease, such as a CRISPR/Cas system and optionally a gRNA. In some embodiments, the system comprises an engineered nuclease. In some embodiments, the system comprises a site-directed nuclease. In some embodiments, the site-directed nuclease comprises a CRISPR/Cas nuclease system. In some embodiments, the Cas nuclease is Cas9. In some embodiments, the nuclease is Mad7. In some embodiments, the guide RNA comprising the CRISPR/Cas system is an sgRNA.

### CRISPR/Cas Nuclease Systems

Naturally-occurring CRISPR/Cas systems are genetic defense systems that provides a form of acquired immunity in prokaryotes. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see e.g., Koonin et al., (2017) Curr Opin Microbiol 37:67-78).

Engineered versions of CRISPR/Cas systems has been developed in numerous formats to mutate or edit genomic DNA of cells from other species. The general approach of using the CRISPR/Cas system involves the heterologous expression or introduction of a site-directed nuclease (e.g.: Cas nuclease) in combination with a guide RNA (gRNA) into a cell, resulting in a DNA cleavage event (e.g., the formation a single-strand or double-strand break (SSB or DSB)) in the backbone of the cell's genomic DNA at a precise, targetable location. The manner in which the DNA cleavage event is repaired by the cell provides the opportunity to edit the genome by the addition, removal, or modification (substitution) of DNA nucleotide(s) or sequences (e.g., genes).

i. Guide RNAs (gRNAs)

Engineered CRISPR/Cas systems comprise at least two components: 1) a guide RNA (gRNA) molecule and 2) a Cas nuclease, which interact to form a gRNA/Cas nuclease complex. A gRNA comprises at least a user-defined targeting domain termed a “spacer” comprising a nucleotide sequence and a CRISPR repeat sequence. In engineered CRISPR/Cas systems, a gRNA/Cas nuclease complex is targeted to a specific target sequence of interest within a target nucleic acid (e.g., a genomic DNA molecule) by generating a gRNA comprising a spacer with a nucleotide sequence that is able to bind to the specific target sequence in a complementary fashion (See Jinek et al., Science, 337, 816-821 (2012) and Deltcheva et al., Nature, 471, 602-607 (2011)). Thus, the spacer provides the targeting function of the gRNA/Cas nuclease complex.

In naturally-occurring type II-CRISPR/Cas systems, the “gRNA” is comprised of two RNA strands: 1) a CRISPR RNA (crRNA) comprising the spacer and CRISPR repeat sequence, and 2) a trans-activating CRISPR RNA (tracrRNA). In Type II-CRISPR/Cas systems, the portion of the crRNA comprising the CRISPR repeat sequence and a portion of the tracrRNA hybridize to form a crRNA:tracrRNA duplex, which interacts with a Cas nuclease (e.g., Cas9). As used herein, the terms “split gRNA” or “modular gRNA” refer to a gRNA molecule comprising two RNA strands, wherein the first RNA strand incorporates the crRNA function(s) and/or structure and the second RNA strand incorporates the tracrRNA function(s) and/or structure, and wherein the first and second RNA strands partially hybridize.

Accordingly, in some embodiments, a gRNA comprises two RNA molecules. In some embodiments, the gRNA comprises a CRISPR RNA (crRNA) and a trans-activating CRISPR RNA (tracrRNA). In some embodiments, the gRNA is a split gRNA. In some embodiments, the gRNA is a modular gRNA. In some embodiments, the split gRNA comprises a first strand comprising, from 5′ to 3′, a spacer, and a first region of complementarity; and a second strand comprising, from 5′ to 3′, a second region of complementarity; and optionally a tail domain.

In some embodiments, the crRNA comprises a spacer comprising a nucleotide sequence that is complementary to and hybridizes with a sequence that is complementary to the target sequence on a target nucleic acid (e.g., a genomic DNA molecule). In some embodiments, the crRNA comprises a region that is complementary to and hybridizes with a portion of the tracrRNA.

In some embodiments, the tracrRNA may comprise all or a portion of a wild-type tracrRNA sequence from a naturally-occurring CRISPR/Cas system. In some embodiments, the tracrRNA may comprise a truncated or modified variant of the wild-type tracr RNA. The length of the tracr RNA may depend on the CRISPR/Cas system used. In some embodiments, the tracrRNA may comprise 5, 6, 7, 8, 9, 10, 11, 12, 13, 14, 15, 16, 17, 18, 19, 20, 25, 30, 40, 50, 60, 70, 80, 90, 100, or more than 100 nucleotides in length. In certain embodiments, the tracrRNA is at least 26 nucleotides in length. In additional embodiments, the tracrRNA is at least 40 nucleotides in length. In some embodiments, the tracrRNA may comprise certain secondary structures, such as, e.g., one or more hairpins or stem-loop structures, or one or more bulge structures.

Single Guide RNA (sgRNA)

Engineered CRISPR/Cas nuclease systems often combine a crRNA and a tracrRNA into a single RNA molecule, referred to herein as a “single guide RNA” (sgRNA), by adding a linker between these components. Without being bound by theory, similar to a duplexed crRNA and tracrRNA, an sgRNA will form a complex with a Cas nuclease (e.g., Cas9), guide the Cas nuclease to a target sequence and activate the Cas nuclease for cleavage the target nucleic acid (e.g., genomic DNA). Accordingly, in some embodiments, the gRNA may comprise a crRNA and a tracrRNA that are operably linked. In some embodiments, the sgRNA may comprise a crRNA covalently linked to a tracrRNA. In some embodiments, the crRNA and the tracrRNA is covalently linked via a linker. In some embodiments, the sgRNA may comprise a stem-loop structure via base pairing between the crRNA and the tracrRNA. In some embodiments, a sgRNA comprises, from 5′ to 3′, a spacer, a first region of complementarity, a linking domain, a second region of complementarity, and, optionally, a tail domain.

The sgRNA can be unmodified or modified. For example, modified sgRNAs can comprise one or more 2′-O-methyl phosphorothioate nucleotides.

By way of illustration, guide RNAs used in the CRISPR/Cas system, or other smaller RNAs can be readily synthesized by chemical means, as illustrated herein and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

### Spacers

In some embodiments, the gRNAs comprise a spacer sequence. A spacer sequence is a sequence that defines the target site of a target nucleic acid (e.g. DNA). The target nucleic acid is a double-stranded molecule: one strand comprises the target sequence adjacent to a PAM sequence and is referred to as the “PAM strand,” and the second strand is referred to as the “non-PAM strand” and is complementary to the PAM strand and target sequence. Both gRNA spacer and the target sequence are complementary to the non-PAM strand of the target nucleic acid. In some embodiments, a spacer sequence corresponding to a target sequence adjacent to a PAM sequence is complementary to the non-PAM strand of the target nucleic acid. Thus, in some embodiments, a spacer sequence which corresponds to a target sequence adjacent to a PAM sequence is identical to the PAM strand. The gRNA spacer sequence hybridizes to the complementary strand (e.g.: the non-PAM strand of the target nucleic acid/target site). In some embodiments, the spacer is sufficiently complementary to the complementary strand of the target sequence (e.g.: non-PAM strand), as to target a Cas nuclease to the target nucleic acid. In some embodiments, the spacer is at least 80%, 85%, 90% or 95% complementary to the non-PAM strand of the target nucleic acid. In some embodiments, the spacer is 100% complementary to the non-PAM strand of the target nucleic acid. In some embodiments, the spacer comprises 1, 2, 3, 4, 5, 6 or more nucleotides that are not complementary with the non-PAM strand of the target nucleic acid. In some embodiments, the spacer comprises 1 nucleotide that is not complementary with the non-PAM strand of the target nucleic acid. In some embodiments, the spacer comprises 2 nucleotides that are not complementary with the non-PAM strand of the target nucleic acid.

In some embodiments, the 5′ most nucleotide of gRNA comprises the 5′ most nucleotide of the spacer. In some embodiments, the spacer is located at the 5′ end of the crRNA. In some embodiments, the spacer is located at the 5′ end of the sgRNA. In some embodiments, the spacer is about 15-50, about 20-45, about 25-40 or about 30-35 nucleotides in length. In some embodiments, the spacer is about 19-22 nucleotides in length. In some embodiments the spacer is about 15, 16, 17, 18, 19, 20, 21, 22, 23, 24, 25, 26, 27, 28, 29 or 30 nucleotides in length. In some embodiments the spacer is 19 nucleotides in length. In some embodiments, the spacer is 20 nucleotides in length, in some embodiments, the spacer is 21 nucleotides in length.

In some embodiments, the nucleotide sequence of the spacer is designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, and/or presence of SNPs.

In some embodiments, the spacer comprise at least one or more modified nucleotide(s) such as those described herein. The disclosure provides gRNA molecules comprising a spacer which may comprise the nucleobase uracil (U), while any DNA encoding a gRNA comprising a spacer comprising the nucleobase uracil (U) will comprise the nucleobase thymine (T) in the corresponding position(s).

ii. Methods of Making gRNAs

Methods for making gRNAs are known to those of skill in the art and include but not limited to in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in International Application PCT/US2013/30062. Accordingly, the present disclosure also includes polynucleotides, e.g., DNA, constructs and vectors are used to in vitro transcribe a gRNA described herein.

In some embodiments, non-natural modified nucleobases are introduced into polynucleotides, e.g., gRNA, during synthesis or post-synthesis. In certain embodiments, modifications are on internucleoside linkages, purine or pyrimidine bases, or sugar. In some embodiments, the modification is introduced at the terminal of a polynucleotide; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

In some embodiments, enzymatic or chemical ligation methods are used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

In some embodiments, the disclosure provides nucleic acids, e.g., vectors, encoding gRNAs described herein. In some embodiments, the nucleic acid is a DNA molecule. In other embodiments, the nucleic acid is an RNA molecule. In some embodiments, the nucleic acid comprises a nucleotide sequence encoding a crRNA. In some embodiments, the nucleotide sequence encoding the crRNA comprises a spacer flanked by all or a portion of a repeat sequence from a naturally-occurring CRISPR/Cas system. In some embodiments, the nucleic acid comprises a nucleotide sequence encoding a tracrRNA. In some embodiments, the crRNA and the tracrRNA is encoded by two separate nucleic acids. In other embodiments, the crRNA and the tracrRNA is encoded by a single nucleic acid. In some embodiments, the crRNA and the tracrRNA is encoded by opposite strands of a single nucleic acid. In other embodiments, the crRNA and the tracrRNA is encoded by the same strand of a single nucleic acid.

In some embodiments, the gRNAs provided by the disclosure are chemically synthesized by any means described in the art (see e.g., WO/2005/01248). While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together.

In some embodiments, more than one guide RNA can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

The guide RNA may target any sequence of interest via the targeting sequence (e.g., spacer sequence) of the crRNA. In some embodiments, the degree of complementarity between the targeting sequence of the guide RNA and the target sequence on the target nucleic acid molecule is about 60%, 65%, 70%, 75%, 80%, 85%, 90%, 95%, 97%, 98%, 99%, or 100%. In some embodiments, the targeting sequence of the guide RNA and the target sequence on the target nucleic acid molecule is 100% complementary. In other embodiments, the targeting sequence of the guide RNA and the target sequence on the target nucleic acid molecule may contain at least one mismatch. For example, the targeting sequence of the guide RNA and the target sequence on the target nucleic acid molecule may contain 1, 2, 3, 4, 5, 6, 7, 8, 9, or 10 mismatches. In some embodiments, the targeting sequence of the guide RNA and the target sequence on the target nucleic acid molecule may contain 1-6 mismatches. In some embodiments, the targeting sequence of the guide RNA and the target sequence on the target nucleic acid molecule may contain 5 or 6 mismatches.

The length of the targeting sequence may depend on the CRISPR-Cas system and components used. For example, different Cas9 proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence may comprise 18-24 nucleotides in length. In some embodiments, the targeting sequence may comprise 19-21 nucleotides in length. In some embodiments, the targeting sequence may comprise 20 nucleotides in length.

In some embodiments of the present disclosure, a CRISPR/Cas nuclease system includes at least one guide RNA. In some embodiments, the guide RNA and the Cas protein may form a ribonucleoprotein (RNP), e.g., a CRISPR/Cas complex. The guide RNA may guide the Cas protein to a target sequence on a target nucleic acid molecule (e.g., a genomic DNA molecule), where the Cas protein cleaves the target nucleic acid. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex. In some embodiments, the CRISPR/Cas complex is an engineered Class 2 Type V CRISPR system. In some embodiments, the endonuclease is Mad7.

iii. Cas Nuclease

In some embodiments, the disclosure provides compositions and systems (e.g., an engineered CRISPR/Cas system) comprising a site-directed nuclease, wherein the site-directed nuclease is a Cas nuclease. The Cas nuclease may comprise at least one domain that interacts with a guide RNA (gRNA). Additionally, the Cas nuclease are directed to a target sequence by a guide RNA. The guide RNA interacts with the Cas nuclease as well as the target sequence such that, once directed to the target sequence, the Cas nuclease is capable of cleaving the target sequence. In some embodiments, the guide RNA provides the specificity for the cleavage of the target sequence, and the Cas nuclease are universal and paired with different guide RNAs to cleave different target sequences.

In some embodiments, the CRISPR/Cas system comprise components derived from a Type-I, Type-II, or Type-HI system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163.1-13) is homologous to Cas9, and contains a RuvC-like nuclease domain.

In some embodiments, the Cas nuclease are from a Type-II CRISPR/Cas system (e.g., a Cas9 protein from a CRISPR/Cas9 system). In some embodiments, the Cas nuclease are from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

A Type-II CRISPR/Cas system component are from a Type-IIA, Type-IIB, or Type-IIC system. Cas9 and its orthologs are encompassed. Non-limiting exemplary species that the Cas9 nuclease or other components are from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina. In some embodiments, the Cas9 protein are from Streptococcus pyogenes (SpCas9). In some embodiments, the Cas9 protein are from Streptococcus thermophilus (StCas9). In some embodiments, the Cas9 protein are from Neisseria meningitides (NmCas9). In some embodiments, the Cas9 protein are from Staphylococcus aureus (SaCas9). In some embodiments, the Cas9 protein are from Campylobacter jejuni (CjCas9).

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease). In some embodiments, the nuclease system described herein comprises a nickase and a pair of guide RNAs that are complementary to the sense and antisense strands of the target sequence, respectively. The guide RNAs directs the nickase to target and introduce a DSB by generating a nick on opposite strands of the target sequence (i.e., double nicking). Chimeric Cas9 nucleases are used, where one domain or region of the protein is replaced by a portion of a different protein. For example, a Cas9 nuclease domain is replaced with a domain from a different nuclease such as Fok1. A Cas9 nuclease is a modified nuclease.

In some embodiments, the Cas nuclease is from a Type-1 CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

In some embodiments, the Cas nuclease is a Mad endonuclease. CRISPR/Mad systems are closely related to the Type V (Cpf1-like) of Class-2 family of Cas enzymes. In some embodiments, the CRISPR-Mad system employs an Eubacterium rectale Mad7 endonuclease or variant thereof. The Mad7-crRNA complex cleaves target DNA by identification of a PAM 5′-YTTN.

### Engineered Nucleases

In some embodiments, the cells described herein are genetically engineered with a site-directed nuclease, wherein the site-directed nuclease is an engineered nuclease. Exemplary engineered nucleases are meganuclease (e.g., homing endonucleases), ZFN, TALEN, and megaTAL.

Naturally-occurring meganucleases may recognize and cleave double-stranded DNA sequences of about 12 to 40 base pairs and are commonly grouped into five families. In some embodiments, the meganuclease are chosen from the LAGLIDADG family, the GIY-YIG family, the HNH family, the His-Cys box family, and the PD-(D/E)XK family. In some embodiments, the DNA binding domain of the meganuclease are engineered to recognize and bind to a sequence other than its cognate target sequence. In some embodiments, the DNA binding domain of the meganuclease are fused to a heterologous nuclease domain. In some embodiments, the meganuclease, such as a homing endonuclease, are fused to TAL modules to create a hybrid protein, such as a “megaTAL” protein. The megaTAL protein have improved DNA targeting specificity by recognizing the target sequences of both the DNA binding domain of the meganuclease and the TAL modules.

ZFNs are fusion proteins comprising a zinc-finger DNA binding domain (“zinc fingers” or “ZFs”) and a nuclease domain. Each naturally-occurring ZF may bind to three consecutive base pairs (a DNA triplet), and ZF repeats are combined to recognize a DNA target sequence and provide sufficient affinity. Thus, engineered ZF repeats are combined to recognize longer DNA sequences, such as, e.g., 9-, 12-, 15-, or 18-bp, etc. In some embodiments, the ZFN comprise ZFs fused to a nuclease domain from a restriction endonuclease. For example, the restriction endonuclease is FokI. In some embodiments, the nuclease domain comprises a dimerization domain, such as when the nuclease dimerizes to be active, and a pair of ZFNs comprising the ZF repeats and the nuclease domain is designed for targeting a target sequence, which comprises two half target sequences recognized by each ZF repeats on opposite strands of the DNA molecule, with an interconnecting sequence in between (which is sometimes called a spacer in the literature). For example, the interconnecting sequence is 5 to 7 bp in length. When both ZFNs of the pair bind, the nuclease domain may dimerize and introduce a DSB within the interconnecting sequence. In some embodiments, the dimerization domain of the nuclease domain comprises a knob-into-hole motif to promote dimerization. For example, the ZFN comprises a knob-into-hole motif in the dimerization domain of FokI.

The DNA binding domain of TALENs usually comprises a variable number of 34 or 35 amino acid repeats (“modules” or “TAL modules”), with each module binding to a single DNA base pair, A, T, G, or C. Adjacent residues at positions 12 and 13 (the “repeat-variable di-residue” or RVD) of each module specify the single DNA base pair that the module binds to. Though modules used to recognize G may also have affinity for A, TALENs benefit from a simple code of recognition—one module for each of the 4 bases—which greatly simplifies the customization of a DNA-binding domain recognizing a specific target sequence. In some embodiments, the TALEN may comprise a nuclease domain from a restriction endonuclease. For example, the restriction endonuclease is FokI. In some embodiments, the nuclease domain may dimerize to be active, and a pair of TALENS is designed for targeting a target sequence, which comprises two half target sequences recognized by each DNA binding domain on opposite strands of the DNA molecule, with an interconnecting sequence in between. For example, each half target sequence is in the range of 10 to 20 bp, and the interconnecting sequence is 12 to 19 bp in length. When both TALENs of the pair bind, the nuclease domain may dimerize and introduce a DSB within the interconnecting sequence. In some embodiments, the dimerization domain of the nuclease domain may comprise a knob-into-hole motif to promote dimerization. For example, the TALEN may comprise a knob-into-hole motif in the dimerization domain of FokI.

### Target Sites

In some embodiments, the site-directed nucleases described herein are directed to and cleave (e.g., introduce a DSB) a target nucleic acid molecule. In some embodiments, the target nucleic acid molecule is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target nucleic acid molecule is a blood-lineage gene. In some embodiments, the blood-lineage gene is protein tyrosine phosphatase receptor type C (PTPRC), IL2RG, or IL2RB. In some embodiments, the target nucleic acid is a gene associated with rapamycin response. In some embodiments, the target nucleic acid is FKBP12. In some embodiments, the target nucleic acid is B2M, TRAC or SIRPA.

The target nucleic acid molecule is any DNA molecule that is endogenous or exogenous to a cell. As used herein, the term “endogenous sequence” refers to a sequence that is native to the cell. In some embodiments, the target nucleic acid molecule is a genomic DNA (gDNA) molecule or a chromosome from a cell or in the cell. In some embodiments, the target sequence of the target nucleic acid molecule is a genomic sequence from a cell or in the cell. In some embodiments, the target sequence may be located in a coding sequence of a gene, an intron sequence of a gene, a transcriptional control sequence of a gene, a translational control sequence of a gene, or a non-coding sequence between genes. In some embodiments, the gene may be a protein coding gene. In other embodiments, the gene may be a non-coding RNA gene. In some embodiments, the target sequence may comprise all or a portion of a disease-associated gene.

In some embodiments, the target sequence may be located in a non-genic functional site in the genome that controls aspects of chromatin organization, such as a scaffold site or locus control region. In some embodiments, the target sequence may be a genetic safe harbor site, i.e., a locus that facilitates safe genetic modification.

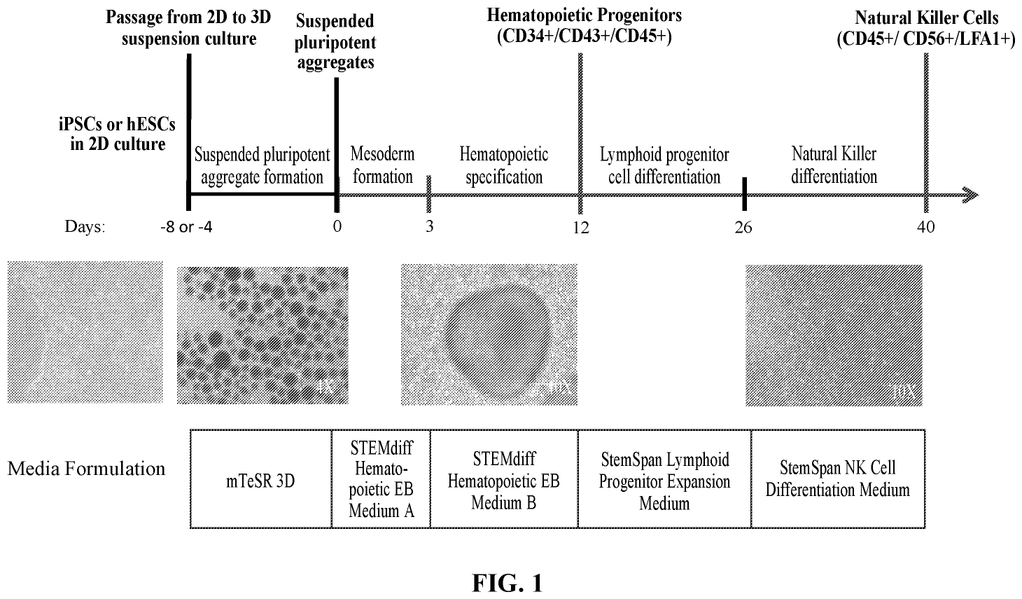

In some embodiments, the target sequence may be adjacent to a protospacer adjacent motif (PAM), a short sequence recognized by a CRISPR/Cas complex. In some embodiments, the PAM may be adjacent to or within 1, 2, 3, or 4, nucleotides of the 3′ end of the target sequence. In some embodiments, the target sequence may include the PAM. The length and the sequence of the PAM may depend on the Cas protein used. For example, the PAM may be selected from a consensus or a particular PAM sequence for a specific Cas nuclease or Cas ortholog, including those disclosed in FIG. 1 of Ran et al., (2015) Nature, 520:186-191 (2015), which is incorporated herein by reference. In some embodiments, the PAM may comprise 2, 3, 4, 5, 6, 7, 8, 9, or 10 nucleotides in length. Non-limiting exemplary PAM sequences include NGG (SpCas9 WT, SpCas9 nickase, dimeric dCas9-Fok1, SpCas9-HF1, SpCas9 K855A, eSpCas9 (1.0), eSpCas9 (1.1)), NGAN or NGNG (SpCas9 VQR variant), NGAG (SpCas9 EQR variant), NGCG (SpCas9 VRER variant), NAAG (SpCas9 QQR1 variant), NNGRRT or NNGRRN (SaCas9), NNNRRT (KKH SaCas9), NNNNRYAC (CjCas9), NNAGAAW (StlCas9), NAAAAC (TdCas9), NGGNG (St3Cas9), NG (FnCas9), NAAAAN (TdCas9), NNAAAAW (StCas9), NNNNACA (CjCas9), GNNNCNNA (PmCas9), and NNNNGATT (NmCas9) (see e.g., Cong et al., (2013) Science 339:819-823; Kleinstiver et al., (2015) Nat Biotechnol 33:1293-1298; Kleinstiver et al., (2015) Nature 523:481-485; Kleinstiver et al., (2016) Nature 529:490-495; Tsai et al., (2014) Nat Biotechnol 32:569-576; Slaymaker et al., (2016) Science 351:84-88; Anders et al., (2016) Mol Cell 61:895-902; Kim et al., (2017) Nat Comm 8:14500; Fonfara et al., (2013) Nucleic Acids Res 42:2577-2590; Garneau et al., (2010) Nature 468:67-71; Magadan et al., (2012) PLoS ONE 7:e40913; Esvelt et al., (2013) Nat Methods 10(11):1116-1121 (wherein N is defined as any nucleotide, W is defined as either A or T, R is defined as a purine (A) or (G), and Y is defined as a pyrimidine (C) or (T)). In some embodiments, the PAM sequence is NGG. In some embodiments, the PAM sequence is NGAN. In some embodiments, the PAM sequence is NGNG. In some embodiments, the PAM is NNGRRT. In some embodiments, the PAM sequence is NGGNG. In some embodiments, the PAM sequence may be NNAAAAW.

### Ribonucleoproteins

In some embodiments, the site-directed polypeptide (e.g., Cas nuclease) and genome-targeting nucleic acid (e.g., gRNA or sgRNA) may each be administered separately to a cell or a subject. In some embodiments, the site-directed polypeptide may be pre-complexed with one or more guide RNAs, or one or more sgRNAs. Such pre-complexed material is known as a ribonucleoprotein particle (RNP). In some embodiments, the nuclease system comprises a ribonucleoprotein (RNP). In some embodiments, the nuclease system comprises a Cas9 RNP comprising a purified Cas9 protein in complex with a gRNA. In some embodiments, the nuclease system comprises a Mad7 RNP comprising a purified Mad7 protein in complex with a gRNA. Cas9 and Mad7 protein can be expressed and purified by any means known in the art. Ribonucleoproteins are assembled in vitro and can be delivered directly to cells using standard electroporation or transfection techniques known in the art.

### Engineered Stem Cells

In some embodiments, the disclosure provides engineered stem cells transiently or stably expressing a synthetic cytokine receptor complex. In some embodiments, the disclosure provides engineered stem cells stably expressing a synthetic cytokine receptor complex.

In some embodiments, the engineered stem cells comprise a genome comprising a nucleotide sequence encoding a synthetic cytokine receptor complex. In some embodiments, the genome further comprises a disrupted B2M, TRAC, and/or SIRPA locus. In some embodiments, the genome further comprises a disrupted FKBP12 locus.

In some embodiments, the engineered stem cells comprise a genome comprising (i) a nucleotide sequence encoding a synthetic cytokine receptor complex, (ii) a disrupted B2M locus, and (iii) a disrupted FKBP12 locus. In some embodiments, the engineered stem cells comprise a genome comprising (i) a nucleotide sequence encoding a synthetic cytokine receptor complex, (ii) a disrupted TRAC locus, and (iii) a disrupted FKBP12 locus. In some embodiments, the engineered stem cells comprise a genome comprising (i) a nucleotide sequence encoding a synthetic cytokine receptor complex, (ii) a disrupted SIRPA locus, and (iii) a disrupted FKBP12 locus. In some embodiments, the engineered stem cells comprise a genome comprising (i) a nucleotide sequence encoding a synthetic cytokine receptor complex, (ii) a disrupted B2M locus, (iii) a disrupted TRAC locus, and (iv) a disrupted FKBP12 locus. In some embodiments, the engineered stem cells comprise a genome comprising (i) a nucleotide sequence encoding a synthetic cytokine receptor complex, (ii) a disrupted B2M locus, (iii) a disrupted SIRPA locus, and (iv) a disrupted FKBP12 locus. In some embodiments, the engineered stem cells comprise a genome comprising (i) a nucleotide sequence encoding a synthetic cytokine receptor complex, (ii) a disrupted SIRPA locus, (iii) a disrupted TRAC locus, and (iv) a disrupted FKBP12 locus.

### Engineered Cells

In some embodiments, the cell populations described herein are genetically engineered. In some embodiments, the source cells are genetically engineered. In some embodiments, the mesoderm cells are genetically engineered. In some embodiments, the embryoid body cells are genetically engineered. In some embodiments, the hematopoietic progenitor cells are genetically engineered. In some embodiments, the differentiated NK cells are genetically engineered. In some embodiments, the mature NK cells are genetically engineered. In some embodiments, genetic engineering reduces expression of an endogenous gene. In some embodiments, genetic engineering increases expression of an endogenous gene.

In some embodiments, genetically engineering a cell comprises introducing foreign DNA into the cell. In some embodiments, the foreign DNA is a gene. In some embodiments, the foreign DNA alters expression of endogenous genes.

In some embodiments, genetic engineering comprises introducing RNA into the cell, such as interfering RNAs (RNAi), Double-stranded ma (dsrna), small interfering RNAs (siRNAs), and/or microrna (miRNA).

In some embodiments, genetic engineering comprises introducing DNA into the cell, such as a plasmid or a bacterial artificial chromosome (BAC).

In some embodiments, genetic engineering comprises introducing: (a) a fusion protein comprising a DNA-targeting protein and a nuclease or (b) an RNA-guided nuclease. For example, in some embodiments, the DNA-targeting protein or RNA-guided nuclease comprises a zinc finger protein (ZFP), a TAL protein, or a clustered regularly interspaced short palindromic nucleic acid (CRISPR) specific for the gene. In some embodiments, the disruption comprises introducing a zinc finger nuclease (ZFN), a TAL-effector nuclease (TALEN), or and a CRISPR-Cas9 combination that specifically binds to, recognizes, or hybridizes to the gene. In some embodiments, the introducing is carried out by introducing into the cell a nucleic acid comprising a sequence encoding the DNA-binding protein, DNA-binding nucleotide, and/or complex comprising the DNA-binding protein or DNA-binding nucleotide. In some embodiments, the nucleic acid is a viral vector.

In some embodiments, a genetically engineered cell described herein comprises a chimeric antigen receptor (CAR). In some embodiments, a genetically engineered stem cell comprises a CAR. In some embodiments, a genetically engineered hematopoietic progenitor comprises a CAR. In some embodiments, a genetically engineered NK cell comprises a CAR.

In some embodiments, a genetically engineered cell described herein comprises a rapamycin-activated cytokine receptor (RACR). In some embodiments, a genetically engineered stem cell comprises a RACR. In some embodiments, a genetically engineered hematopoietic progenitor comprises a RACR. In some embodiments, a genetically engineered NK cell comprises a RACR.

In some embodiments, a genetically engineered cell described herein comprises a CAR and a RACR. In some embodiments, a genetically engineered stem cell comprises a CAR and a RACR. In some embodiments, a genetically engineered hematopoietic progenitor comprises a CAR and a RACR. In some embodiments, a genetically engineered NK cell comprises a CAR and a RACR.

In some embodiments, the genetically engineered NK cells may comprise an inactivating mutation. In some embodiments, an inactivating mutation is a nonsense mutation. In some embodiments, the nonsense mutation is a premature stop codon. In some embodiments, an inactivating mutation is a missense mutation.

### Synthetic Cytokine Receptor Complex

In some embodiments, a cell described herein is genetically engineered to express a synthetic cytokine receptor. In some embodiments, a synthetic cytokine receptor comprises a synthetic gamma chain and a synthetic beta chain, each comprising a dimerization domain. The dimerization domains controllable dimerize in the present of a non-physiological ligand, thereby activating signaling the synthetic cytokine receptor.

The synthetic gamma chain polypeptide comprises a first dimerization domain, a first transmembrane domain, and an intracellular domain. In some embodiments, the intracellular domain is an interleukin-2 receptor subunit gamma (IL-2RG) intracellular domain. The dimerization domain may be extracellular (N-terminal to the transmembrane domain) or intracellular (C-terminal to the transmembrane domain) and N- or C-terminal to the IL-2G intracellular domain.

The synthetic beta chain polypeptide comprises a second dimerization domain, a second transmembrane domain, and an intracellular domain. In some embodiments, the intracellular domain is selected from an interleukin-2 receptor subunit beta (IL-2RB) intracellular domain, an interleukin-7 receptor subunit beta (IL-7RB) intracellular domain, or an interleukin-21 receptor subunit beta (IL-21RB) intracellular domain. In some embodiments, the intracellular domain comprises an interleukin-2/interleukin-15 receptor subunit beta (IL-2/15RB). In some embodiments, the intracellular domain comprises an interleukin-15 receptor alpha subunit. The synthetic gamma chain polypeptide comprises a first dimerization domain, a first transmembrane domain, and an interleukin-2 receptor subunit gamma (IL-2RG) intracellular domain. The dimerization domain may be extracellular (N-terminal to the transmembrane domain) or intracellular (C-terminal to the transmembrane domain and N- or C-terminal to the IL-2RB or IL-7RB intracellular domain).

The non-physiological ligand may activate the synthetic cytokine receptor in the cytotoxic innate lymphoid cells to induce expansion and/or activation of the engineered cytotoxic innate lymphoid cells. In a preferred embodiment, the non-physiological ligand is rapamycin or a rapalog, such synthetic cytokine receptor termed a rapamycin-activated cytokine receptor (RACR).

In some embodiments, the non-physiological ligand activates the synthetic cytokine receptor in the NK cells to induce expansion of the NK cells. In some embodiments, the activation of the synthetic cytokine receptor results in at least about 10-fold, at least about 50-fold, at least about 100-fold, at least about 200-fold, at least about 300-fold, at least about 400-fold, at least about 500-fold, at least about 1000-fold, at least about 1500-fold, at least about 2000-fold, at least about 2500-fold, at least about 3000-fold, at least about 3500-fold, or at least about 4000-fold increased number of NK cells compared to uninduced cells.

In some embodiments, the NK cells increase by about 10-fold to about 100-fold, about 50-fold to about 200-fold, about 100-fold to about 300-fold, about 200-fold to about 400-fold, about 300-fold to about 500-fold, about 400-fold to about 1000-fold, about 500-fold to about 1500-fold, about 1000-fold to about 2000-fold, about 1500-fold to about 2500-fold, about 2000-fold to about 3000-fold, about 2500-fold to about 3500-fold, about 3000-fold to about 4000-fold, or any value in between these ranges.

### Intracellular Domain

In some embodiments, the intracellular signaling domain of the first transmembrane receptor protein comprises an interleukin-2 receptor subunit gamma (IL2Rg) domain. In some embodiments, the IL2Rg domain comprises the sequence set forth in SEQ ID NO: 1. In some embodiments, the IL2Rg Common Gamma Chain Intracellular domain has at least 80% amino acid identity, at least 85% amino acid identity, at least 90% amino acid identity, at least 95% amino acid identity, or 100% amino acid identity to SEQ ID NO: 1.

In some embodiments, the sequence of a IL2RG Common Gamma Chain Intracellular domain is set forth in SEQ ID NO: 1:

ERTMPRIPTLKNLEDLVTEYHGNFSAWSGVSKGLAESLQPDYSERLCLV
SEIPPKGGALGEGPGASPCNQHSPYWAPPCYTLKPET.

In some embodiments, the synthetic cytokine receptor comprises a first transmembrane receptor protein comprising an IL-2RG intracellular domain, a first dimerization domain, a second transmembrane receptor protein comprising an IL-2RB intracellular domain, and a second dimerization domain.

In some embodiments, the synthetic beta chain comprises an interleukin-2 receptor subunit beta (IL2RB) intracellular domain. In some embodiments, the IL-2 receptor subunit beta is referred to as the IL-2/IL-15 receptor beta subunit. In some embodiments, the IL2RB intracellular domain comprises the sequence set forth in SEQ ID NO: 2. In some embodiments, the IL2RB intracellular domain has at least 80% amino acid identity, at least 85% amino acid identity, at least 90% amino acid identity, at least 95% amino acid identity, or 100% amino acid identity to SEQ ID NO: 2.

In some embodiments, the sequence of a IL2RB intracellular domain is set forth in SEQ ID NO: 2:

NCRNTGPWLKKVLKCNTPDPSKFFSQLSSEHGGDVQKWLSSPFPSSSFS
PGGLAPEISPLEVLERDKVTQLLLQQDKVPEPASLSSNHSLTSCFTNQG
YFFFHLPDALEIEACQVYFTYDPYSEEDPDEGVAGAPTGSSPQPLQPLS
GEDDAYCTFPSRDDLLLFSPSLLGGPSPPSTAPGGSGAGEERMPPSLQE
RVPRDWDPQPLGPPTPGVPDLVDFQPPPELVLREAGEEVPDAGPREGVS
FPWSRPPGQGEFRALNARLPLNTDAYLSLQELQGQDPTHLV

In some embodiments, the synthetic cytokine receptor comprises a first transmembrane receptor protein comprising an IL-2RG intracellular domain, a first dimerization domain, a second transmembrane receptor protein comprising an IL-7RB intracellular domain, and a second dimerization domain.

In some embodiments, the synthetic beta chain comprises an interleukin-7 receptor subunit beta (IL7RB) intracellular domain. In some embodiments, the IL7RB intracellular domain comprises the sequence set forth in SEQ ID NO: 3. In some embodiments, the IL7RB intracellular domain has at least 80% amino acid identity, at least 85% amino acid identity, at least 90% amino acid identity, at least 95% amino acid identity, or 100% amino acid identity to SEQ ID NO: 3.

In some embodiments, the sequence of a IL7RB intracellular domain is set forth in SEQ ID NO: 3:

KKRIKPIVWPSLPDHKKTLEHLCKKPRKNLNVSFNPESFLDCQIHRVDD
IQARDEVEGFLQDTFPQQLEESEKQRLGGDVQSPNCPSEDVVITPESFG
RDSSLTCLAGNVSACDAPILSSSRSLDCRESGKNGPHVYQDLLLSLGTT
NSTLPPPFSLQSGILTLNPVAQGQPILTSLGSNQEEAYVTMSSFYQNQ

In some embodiments, the synthetic cytokine receptor comprises a first transmembrane receptor protein comprising an IL-2RG intracellular domain, a first dimerization domain, a second transmembrane receptor protein comprising an IL-21RB intracellular domain, and a second dimerization domain.

In some embodiments, the synthetic beta chain comprises an interleukin-21 receptor subunit beta (IL21RB) intracellular domain. In some embodiments, the IL21RB intracellular domain comprises the sequence set forth in SEQ ID NO: 4. In some embodiments, the IL21RB intracellular domain has at least 80% amino acid identity, at least 85% amino acid identity, at least 90% amino acid identity, at least 95% amino acid identity, or 100% amino acid identity to SEQ ID NO: 4.

In some embodiments, the sequence of a IL21RB intracellular domain is set forth in SEQ ID NO: 4:

SLKTHPLWRLWKKIWAVPSPERFFMPLYKGCSGDFKKWVGAPFTGSSLE
LGPWSPEVPSTLEVYSCHPPRSPAKRLQLTELQEPAELVESDGVPKPSF
WPTAQNSGGSAYSEERDRPYGLVSIDTVTVLDAEGPCTWPCSCEDDGYP
ALDLDAGLEPSPGLEDPLLDAGTTVLSCGCVSAGSPGLGGPLGSLLDRL
KPPLADGEDWAGGLPWGGRSPGGVSESEAGSPLAGLDMDTFDSGFVGSD
CSSPVECDFTSPGDEGPPRSYLRQWVVIPPPLSSPGPQAS

### Dimerization Domain

The dimerization domains may be heterodimerization domains, including but not limited to FK506-Binding Protein of size 12 kD (FKBP) and a FKBP12-rapamycin binding (FRB) domain, which are known in the art to dimerize in the presence of rapamycin or a rapalog. The FRB domain may comprise a polypeptide sequence at least 80%, at least 90%, at least 95%, at least 96%, at least 97%, at least 98%, at least 99%, or 100% identical to SEQ ID NO: 6 or SEQ ID NO:7. The FKBP domain may comprise a polypeptide sequence at least 80%, at least 90%, at least 95%, at least 96%, at least 97%, at least 98%, at least 99%, or 100% identical to SEQ ID NO: 5.

In some embodiments, the sequence of an illustrative FKBP domain is set forth in SEQ ID NO: 5:

GVQVETISPGDGRTFPKRGQTCVVHYTGMLEDGKKFDSSRDRNKPFKFM
LGKQEVIRGWEEGVAQMSVGQRAKLTISPDYAYGATGHPGIIPPHATLV
FDVELLKLE

In some embodiments, the sequence of an illustrative FRB domain is set forth in SEQ ID NO: 6:

ILWHEMWHEGLEEASRLYFGERNVKGMFEVLEPLHAMMERGPQTLKETS
FNQAYGRDLMEAQEWCRKYMKSGNVKDLTQAWDLYYHVFRRISK

In some embodiments, the sequence of variant FRB domain (FRB mutant domain) is set forth in SEQ ID NO: 7:

ILWHEMWHEGLEEASRLYFGERNVKGMFEVLEPLHAMMERGPQTLKETS
FNQAYGRDLMEAQEWCRKYMKSGNVKDLLQAWDLYYHVFRRISK

Alternatively, the first dimerization domain and the second dimerization domain may be a FK506-Binding Protein of size 12 kD (FKBP) and a calcineurin domain, which are known in the art to dimerize in the presence of FK506 or an analogue thereof.

In some embodiments the dimerization domains are homodimerization domains selected from:
i) FK506-Binding Protein of size 12 kD (FKBP);
ii) cyclophiliA (CypA); or
iii) gyrase B (CyrB);
   with the corresponding non-physiological ligands being, respectively
i) FK1012, AP1510, AP1903, or AP20187;
ii) cyclosporin-A (CsA); or
iii) coumermycin or analogs thereof.

In some embodiments, the first and second dimerization domains of the transmembrane receptor proteins are a FKBP domain and a cyclophilin domain.

In some embodiments, the first and second dimerization domains of the transmembrane receptor proteins are a FKBP domain and a bacterial dihydrofolate reductase (DHFR) domain.

In some embodiments, the first and second dimerization domains of the transmembrane receptor proteins are a calcineurin domain and a cyclophilin domain.

In some embodiments, the first and second dimerization domains of the transmembrane receptor proteins are PYR1-like 1 (PYL1) and abscisic acid insensitive 1 (ABI1).

### Transmembrane Domains

The transmembrane domain is the sequence of the synthetic cytokine receptor that spans the membrane. The transmembrane domain may comprise a hydrophobic alpha helix. In some embodiments, the transmembrane domain is derived from a human protein.

In some embodiments, the sequence of a transmembrane (TM) domain is shown as SEQ ID NO: 8: VVISVGSMGLIISLLCVYFWL

In some embodiments, the sequence of a TM domain is shown as SEQ ID NO: 9:

VAVAGCVFLLISVLLLSGL

In some embodiments, the sequence of TM domain is shown as SEQ ID NO: 10.

PILLTISILSFFSVALLVILACVLW

In some embodiments, the sequence of a TM domain is shown as SEQ ID NO: 11:

GWNPHLLLLLLLVIVFIPAFW

In some embodiments, the sequence of a CD8a signal sequence is shown as SEQ ID NO: 12: MALPVTALLLPLALLLHAARP

### Non-Physiological Ligand

In various embodiments of the compositions and methods of the disclosure, the system comprises a non-physiological ligand. Illustrative small molecules useful as ligands include, without limitation: rapamycin, fluorescein, fluorescein isothiocyanate (FITC), 4-[(6-methylpyrazin-2-yl)oxy]benzoic acid (aMPOB), folate, rhodamine, acetazolamide, and a CA9 ligand.

In some embodiments, the synthetic cytokine receptor is activated by a ligand. In some embodiments, the ligand is a non-physiological ligand.

In some embodiments, the non-physiological ligand is a rapalog.

In some embodiments, the non-physiological ligand is rapamycin.

In some embodiments, the non-physiological ligand is AP21967.

In some embodiments, the non-physiological ligand is FK506.

In some embodiments, the non-physiological ligand is FK1012. In some embodiments, the non-physiological ligand is AP1510. In some embodiments, the non-physiological ligand is AP1903. In some embodiments, the non-physiological ligand is AP20187. In some embodiments, the non-physiological ligand is cyclosporin-A (CsA). In some embodiments, the non-physiological ligand is coumermycin.

In some embodiments, the synthetic cytokine receptor complex activated by folate, fluorescein, aMPOB, acetazolamide, a CA9 ligand, tacrolimus, rapamycin, a rapalog (a rapamycin analog), CD28 ligand, poly(his) tag, Strep-tag, FLAG-tag, VS-tag, Myc-tag, HA-tag, NE-tag, biotin, digoxigenin, dinitrophenol, or a derivative thereof.

In some embodiments, the non-physiological ligand may be an inorganic or organic compound that is less than 1000 Daltons.

In some embodiments, the ligand may be rapamycin or a rapamycin analog (rapalog). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring.

Thus, in some embodiments, the rapalog is everolimus, novolimus, pimecrolimus, ridaforolimus, tacrolimus, temsirolimus, umirolimus, zotarolimus, Temsirolimus (CCI-779), C20-methallylrapamycin, C16-(S)-3-methylindolerapamycin, C16-(S)-3-methylindolerapamycin (C16-iRap), AP21967 (A/C Heterodimerizer, Takara Bio®), sodium mycophenolic acid, benidipine hydrochloride, rapamine, AP23573 (Ridaforolimus), AP1903 (Rimiducid), or metabolites, derivatives, and/or combinations thereof.

In some embodiments, the ligand comprises FK1012 (a semisynthetic dimer of FK506), tacrolimus (FK506), FKCsA (a composite of FK506 and cyclosporine), rapamycin, coumermycin, gibberellin, HaXS dimerizer (chemical dimerizers of HaloTag and SNAP-tag), TMP-HTag (trimethoprim haloenzyme protein dimerizer), or ABT-737 or functional derivatives thereof.

In some embodiments, the non-physiological ligand is present or provided in an amount from 0 nM to 1000 nM such as, e.g., 0.05 nM, 0.1 nM, 0.5. nM, 1.0 nM, 5.0 nM, 10.0 nM, 15.0 nM, 20.0 nM, 25.0 nM, 30.0 nM, 35.0 nM, 40.0 nM, 45.0 nM, 50.0 nM, 55.0 nM, 60.0 nM, 65.0 nM, 70.0 nM, 75.0 nM, 80.0 nM, 90.0 nM, 95.0 nM, 100 nM, 200 nM, 300 nM, 400 nM, 500 nM, 600 nM, 700 nM, 800 nM, 900 nM, or 1000 nM, or an amount that is within a range defined by any two of the aforementioned amounts.

In some embodiments, the non-physiological ligand is AP21967 and is present or provided at 10 nM. In some embodiments, the non-physiological ligand is AP21967 and is present or provided at 20 nM. In some embodiments, the non-physiological ligand is AP21967 and is present or provided at 50 nM. In some embodiments, the non-physiological ligand is AP21967 and is present or provided at 100 nM.

In some embodiments, the non-physiological ligand is rapamycin and is present or provided at 1 nM. In some embodiments, the non-physiological ligand is rapamycin and is present or provided at 10 nM. In some embodiments, the non-physiological ligand is rapamycin and is present or provided at 20 nM. In some embodiments, the non-physiological ligand is rapamycin and is present or provided at 50 nM.

In some embodiments, the non-physiological ligand is a rapalog and is present or provided at 1 nM. In some embodiments, the non-physiological ligand is a rapalog and is present or provided at 10 nM. In some embodiments, the non-physiological ligand is a rapalog and is present or provided at 20 nM. In some embodiments, the non-physiological ligand is a rapalog and is present or provided at 50 nM. In some embodiments, the non-physiological ligand is a rapalog and is present or provided at 100 nM.

In some embodiments, the non-physiological ligand is present or provided at 1 nM. In some embodiments, the non-physiological ligand is present or provided at 10 nM. In some embodiments, the non-physiological ligand is present or provided at 100 nM. In some embodiments, the non-physiological ligand is present or provided at 1000 nM.

### Cytosolic FRB

The FRB domain is an approximately 100 amino acid domain derived from the mTOR protein kinase. It may be expressed in the cytosol as a freely diffusible soluble protein. Advantageously, the FRB domain reduces the inhibitory effects of rapamycin on mTOR in the transduced cells and promote consistent activation of transduced cells giving the cells a proliferative advantage over native cells.

In some embodiments, synthetic cytokine receptor complex comprises a cytosolic polypeptide that binds to the ligand or a complex comprising the ligand.

In some embodiments, the cytosolic polypeptide comprises an FRB domain. In some embodiments, the cytosolic polypeptide comprises an FRB domain and the ligand is rapamycin. The cytosolic FRB domain may comprise a polypeptide sequence at least 80%, at least 90%, at least 95%, at least 96%, at least 97%, at least 98%, at least 99%, or 100% identical to SEQ ID NO: 6 or SEQ ID NO: 7. FRB domain may be a naked FRB domain consisting essentially of a polypeptide having a polypeptide sequence at least 80%, at least 90%, at least 95%, at least 96%, at least 97%, at least 98%, at least 99/o, or 100% identical to SEQ ID NO: 6 or SEQ ID NO: 7. Advantageously, the cytosolic FRB confers resistance to the immunosuppressive effect of the non-physiological ligand (e.g., rapamycin or rapalog).

### Chimeric Antigen Receptor

In some embodiments, a cell described herein is genetically engineered to express a chimeric antigen receptor (CAR).

In some embodiments, the disclosure contemplates a CAR system for use in the treatment of subjects with cancer. In some embodiments, the NK cells of the disclosure comprise a CAR sequence (CAR-NK cells).

In some embodiments, NK cells are engineered to express CAR constructs by transfecting a population of cells with an expression vector encoding the CAR construct. Illustrative examples of populations of cells that may be transfected include HSCs, blood progenitor cells, common lymphoid progenitor cells, or NK cells. Appropriate means for preparing a transduced population of NK cells expressing a selected CAR construct will be well known to the skilled artisan, and includes retrovirus, lentivirus (viral mediated CAR gene delivery system), sleeping beauty, and piggyback (transposon/transposase systems that include a non-viral mediated CAR gene delivery system), to name a few examples. In some embodiments, any of the transduction methods contemplated in the disclosure may be used to generate CAR-expressing NK cells.

### Targeting Agents for CARs

Conventionally, CARs are generated by fusing a polynucleotide encoding a VL, VH, or scFv to the 5′ end of a polynucleotide encoding transmembrane and intracellular domains, and transducing cells with that polynucleotide as well as with the corresponding VH or VL, if needed. Numerous variations on CARs well known in the art and the disclosure contemplates using any of the known variations. Additionally, VL/VH pairs and scFv's for innumerable haptens are known in the art or can be generated by conventional methods routinely. Accordingly, the present disclosure contemplates using any known hapten-binding domain.

Various methods to target CARs and CAR-expressing cells have been described in the art, including, for example in US 2020/0123224, the disclosure of which is incorporated by reference herein. For example, a fluorescein or fluorescein isothiocyanate (FITC) moiety may be conjugated to an agent that binds to a desired target cell (such as a cancer cell), and thereby a CAR-NK cell expressing an anti-fluorescein/FITC chimeric antigen receptor may be selectively targeted to the target cell labeled by the conjugate. In variations, other haptens recognized by CARs may be used in place of fluorescein/FITC. The CAR may be generated using various scFv sequences known in the art, or scFv sequences generated by conventional and routine methods. Further illustrative scFv sequences for fluorescein/FITC and for other haptens are provided in, for example, WO 2021/076788, the disclosure of which is incorporated by reference herein.

In some embodiments, the CAR system of the disclosure makes use of CARs that target a moiety that is not produced or expressed by cells of the subject being treated. This CAR system thus allows for focused targeting of the NK cells to target cells, such as cancer cells. By administration of a small conjugate molecule along with the CAR-expressing NK cells, the NK cell response can be targeted to only those cells expressing the tumor receptor, thereby reducing off-target toxicity, and the activation of NK cells can be more easily controlled due to the rapid clearance of the small conjugate molecule. As an added advantage, the CAR-expressing NK cells can be used as a “universal” cytotoxic cell to target a wide variety of tumors without the need to prepare separate CAR constructs. The targeted moiety recognized by the CAR may also remain constant. It is only the ligand portion of the small conjugate molecule that needs to be altered to allow the system to target cancer cells of different identity.

In one embodiment, the disclosure provides an illustration of this conjugate molecule/CAR system.

In some embodiments, the CAR system of the disclosure utilizes conjugate molecules as the bridge between CAR-expressing cells and targeted cancer cells. The conjugate molecules are conjugates comprising a hapten and a cell-targeting moiety, such as any suitable tumor cell-specific ligand. Illustrative haptens that can be recognized and bound by CARs, include small molecular weight organic molecules such as DNP (2,4-dinitrophenol), TNP (2,4,6-trinitrophenol), biotin, and digoxigenin, along with fluorescein and derivatives thereof, including FITC (fluorescein isothiocyanate), NHS-fluorescein, and pentafluorophenyl ester (PFP) and tetrafluorophenyl ester (TFP) derivatives, a knottin, a centyrin, and a DARPin. Suitable cell-targeting moiety that may themselves act as a hapten for a CAR include knottins (see Kolmar H. et al., The FEBS Journal. 2008. 275(11):26684-90), centyrins, and DARPins (see Reichert, J. M. MAbs 2009. 1(3):190-209).

In some embodiments, the cell-targeting moiety is DUPA (DUPA-(99m) Tc), a ligand bound by PSMA-positive human prostate cancer cells with nanomolar affinity (KD=14 nM; see Kularatne, S. A. et al., Mol Pharm. 2009. 6(3):780-9). In one embodiment, a DUPA derivative can be the ligand of the small molecule ligand linked to a targeting moiety, and DUPA derivatives are described in WO 2015/057852, incorporated herein by reference.

In some embodiments, the cell-targeting moiety is CCK2R ligand, a ligand bound by CCK2R-positive cancer cells (e.g., cancers of the thyroid, lung, pancreas, ovary, brain, stomach, gastrointestinal stroma, and colon; see Wayua. C. et al., Molecular Pharmaceutics. 2013. ePublication).

In some embodiments, the cell-targeting moiety is folate, folic acid, or an analogue thereof, a ligand bound by the folate receptor on cells of cancers that include cancers of the ovary, cervix, endometrium, lung, kidney, brain, breast, colon, and head and neck cancers; see Sega, E. I. et al., Cancer Metastasis Rev. 2008. 27(4):655-64).

In some embodiments, the cell-targeting moiety is an NK-1R ligand. Receptors for NK-1R the ligand are found, for example, on cancers of the colon and pancreas. In some embodiments, the NK-1R ligand may be synthesized according the method disclosed in Int'l Patent Appl. No. PCT/US2015/044229, incorporated herein by reference.

In some embodiments, the cell-targeting moiety may be a peptide ligand, for example, the ligand may be a peptide ligand that is the endogenous ligand for the NK1 receptor. In some embodiments, the small conjugate molecule ligand may be a regulatory peptide that belongs to the family of tachykinins which target tachykinin receptors. Such regulatory peptides include Substance P (SP), neurokinin A (substance K), and neurokinin B (neuromedin K), (see Hennig et al., International Journal of Cancer: 61, 786-792).

In some embodiments, the cell-targeting moiety is a CAIX ligand. Receptors for the CAIX ligand found, for example, on renal, ovarian, vulvar, and breast cancers. The CAIX ligand may also be referred to herein as CA9.

In some embodiments, the cell-targeting moiety is a ligand of gamma glutamyl transpeptidase. The transpeptidase is overexpressed, for example, in ovarian cancer, colon cancer, liver cancer, astrocytic gliomas, melanomas, and leukemias.

In some embodiments, the cell-targeting moiety is a CCK2R ligand. Receptors for the CCK2R ligand found on cancers of the thyroid, lung, pancreas, ovary, brain, stomach, gastrointestinal stroma, and colon, among others.

In one embodiment, the cell-targeting moiety may have a mass of less than about 10,000 Daltons, less than about 9000 Daltons, less than about 8,000 Daltons, less than about 7000 Daltons, less than about 6000 Daltons, less than about 5000 Daltons, less than about 4500 Daltons, less than about 4000 Daltons, less than about 3500 Daltons, less than about 3000 Daltons, less than about 2500 Daltons, less than about 2000 Daltons, less than about 1500 Daltons, less than about 1000 Daltons, or less than about 500 Daltons. In another embodiment, the small molecule ligand may have a mass of about 1 to about 10,000 Daltons, about 1 to about 9000 Daltons, about 1 to about 8,000 Daltons, about 1 to about 7000 Daltons, about 1 to about 6000 Daltons, about 1 to about 5000 Daltons, about 1 to about 4500 Daltons, about 1 to about 4000 Daltons, about 1 to about 3500 Daltons, about 1 to about 3000 Daltons, about 1 to about 2500 Daltons, about 1 to about 2000 Daltons, about 1 to about 1500 Daltons, about 1 to about 1000 Daltons, or about 1 to about 500 Daltons.

In one illustrative embodiment, the linkage in a conjugate described herein can be a direct linkage (e.g., a reaction between the isothiocyanate group of FITC and a free amine group of a small molecule ligand) or the linkage can be through an intermediary linker. In one embodiment, if present, an intermediary linker can be any biocompatible linker known in the art, such as a divalent linker. In one illustrative embodiment, the divalent linker can comprise about 1 to about 30 carbon atoms. In another illustrative embodiment, the divalent linker can comprise about 2 to about 20 carbon atoms. In other embodiments, lower molecular weight divalent linkers (i.e., those having an approximate molecular weight of about 30 to about 300 Da) are employed. In another embodiment, linkers lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In some embodiments, the hapten and the cell-targeting moiety can be directly conjugated through such means as reaction between the isothiocyanate group of FITC and free amine group of small ligands (e.g., folate, DUPA, and CCK2R ligand). However, the use of a linking domain to connect the two molecules may be helpful as it can provide flexibility and stability. Examples of suitable linking domains include: 1) polyethylene glycol (PEG); 2) polyproline; 3) hydrophilic amino acids; 4) sugars; 5) unnatural peptideoglycans; 6) polyvinylpyrrolidone; 7) pluronic F-127. Linker lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In some embodiments, the linker may be a divalent linker that may include one or more spacers.

An illustrative conjugate of the disclosure is FITC-Folate

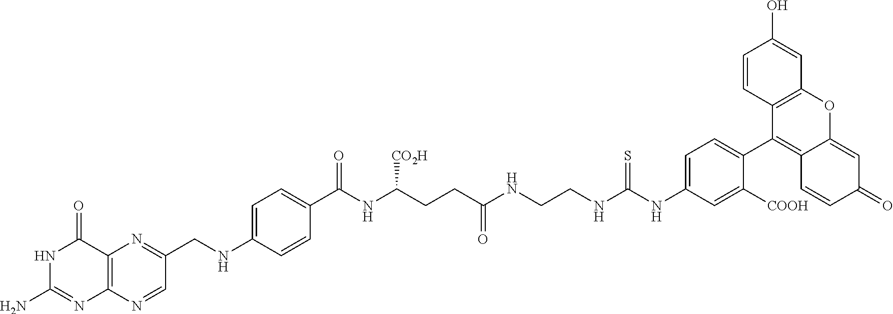 [SMILES: Nc1nc2ncc(CNc3ccc(C(=O)N[C@@H](CCC(=O)NCCNC(=S)Nc4ccc(-c5c6ccc(=O)cc-6oc6cc(O)ccc56)c(C(=O)O)c4)C(=O)O)cc3)nc2c(=O)[nH]1]

An illustrative conjugate of the disclosure is FITC-CA9

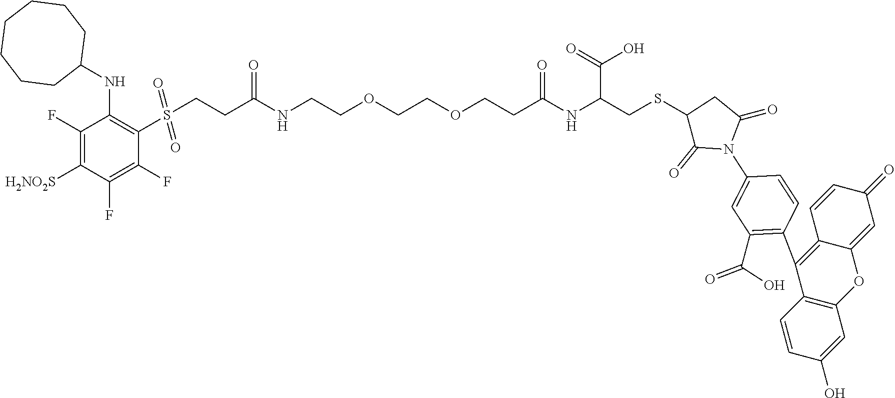 [SMILES: NS(=O)(=O)c1c(F)c(F)c(S(=O)(=O)CCC(=O)NCCOCCOCCC(=O)NC(CSC2CC(=O)N(c3ccc(-c4c5ccc(=O)cc-5oc5cc(O)ccc45)c(C(=O)O)c3)C2=O)C(=O)O)c(NC2CCCCCCC2)c1F]

Illustrative conjugates of the disclosure include the following molecules: FITC-(PEG)-12-Folate, FITC-(PEG)-20-Folate, FITC-(PEG)-108-Folate, FITC-DUPA, FITC-(PEG)12-DUPA, FITC-CCK2R ligand, FITC-(PEG)12-CCK2R ligand, FITC-(PEG)11-NK1R ligand and FITC-(PEG)2-CA9.

While the affinity at which the ligands and cancer cell receptors bind can vary, and in some cases low affinity binding may be preferable (such as about 1 μM), the binding affinity of the ligands and cancer cell receptors will generally be at least about 100 μM, 1 nM, 10 nM, or 100 nM, preferably at least about 1 μM or 10 μM, even more preferably at least about 100 μM.

Examples of conjugates and methods of making them are provided in U.S. patent applications US 2017/0290900, US 2019/0091308, and US 2020/0023009, all of which are incorporated herein by reference.

### CAR Constructs

In some embodiments, the binding portion of the CAR can be, for example, a single chain fragment variable region (scFv) of an antibody, a Fab, Fv, Fc, or (Fab′)2 fragment, and the like. The use of unaltered (i.e., full size) antibodies, such as IgG, IgM, IgA, IgD or IgE, in the CAR or as the CAR is excluded from the scope of the invention.

In some embodiments, a co-stimulation domain serves to enhance the proliferation and survival of the lymphocytes upon binding of the CAR to a targeted moiety. The identity of the co-stimulation domain is limited only in that it has the ability to enhance cellular proliferation and survival activation upon binding of the targeted moiety by the CAR. Suitable co-stimulation domains include, but are not limited to: CD28 (see, e.g., Alvarez-Vallina, L. et al., Eur J Immunol. 1996. 26(10):2304-9); CD137 (4-1BB), a member of the tumor necrosis factor (TNF) receptor family (see, e.g., Imai, C. et al., Leukemia. 2004. 18:676-84); and CD134 (OX40), a member of the TNFR-superfamily of receptors (see, e.g., Latza, U. et al., Eur. J. Immunol. 1994. 24:677). A skilled artisan will understand that sequence variants of these co-stimulation domains can be used, where the variants have the same or similar activity as the domain on which they are modeled. In various embodiments, such variants have at least about 80%, at least about 90%, at least about 95%, at least about 97%, at least about 98%, at least about 99%, or at least about 99.5% sequence identity to the amino acid sequence of the domain from which they are derived.

In some embodiments, the CAR constructs comprise two co-stimulation domains. While the particular combinations include all possible variations of the four noted domains, specific examples include: 1) CD28+CD137 (4-1BB) and 2) CD28+CD134 (OX40).

In some embodiments, the activation signaling domain serves to activate cells upon binding of the CAR to a targeted moiety. The identity of the activation signaling domain is limited only in that it has the ability to induce activation of the selected cell upon binding of the targeted moiety by the CAR. Suitable activation signaling domains include the CD3ζ chain and Fc receptor γ. The skilled artisan will understand that sequence variants of these noted activation signaling domains can be used without adversely impacting the invention, where the variants have the same or similar activity as the domain on which they are modeled. Such variants may have at least about 80%, at least about 90%, at least about 95%, at least about 97%, at least about 98%, at least about 99%, or at least about 99.5% sequence identity to the amino acid sequence of the domain from which they are derived.

In some embodiments, the CARs may include additional elements, such a signal peptide to ensure proper export of the fusion protein to the cells surface, a transmembrane domain to ensure the fusion protein is maintained as an integral membrane protein, and a hinge domain that imparts flexibility to the recognition region and allows strong binding to the targeted moiety.

Illustrative CAR constructs suitable for CAR-NK cells are provided below:
(1) scFv-CD8TM-4-1BBIC-CD3ζs (see, e.g., Liu E, Tong Y, Dotti G, et al., Leukemia. 2018; 32: 520-531);
(2) scFv-CD28TM+IC-CD3ζs (see, e.g., Han J, Chu J, Keung C W et al., Sci Rep. 2015; 5: 11483; Kruschinski A, Moosmann A, Poschke I et al., Proc NatI Acad Sci USA. 2008; 105: 17481-17486; and Chu J, Deng Y, Benson D M et al., Leukemia. 2014; 28: 917-927);
(3) scFv-DAP12TM+IC (see, e.g., Muller N, Michen S, Tietze S et al., J Immunother. 2015; 38: 197-210);
(4) scFv-CD8TM-2B4IC-CD3ζs (see, e.g., Xu Y, Liu Q, Zhong M et al., J Hematol Oncol. 2019; 12: 49);
(5) scFv-2B4TM+IC-CD3ζs (see, e.g., Altvater B, Landmeier S, Pscherer S et al., Clin Cancer Res. 2009; 15: 4857-4866);
(6) scFv-CD28TM+IC-4-1BBIC-CD3ζs (see, e.g., Kloss S, Oberschmidt O, Morgan M et al., Hum Gene Ther. 2017; 28: 897-913);
(7) scFv-CD16TM-2B4IC-CD3ζs (see, e.g., Li Y, Hermanson D L, Moriarity B S Kaufman D S, Cell Stem Cell. 2018; 23: 181-192);
(8) scFv-NKp44TM-DAP10IC-CD3ζs (see, e.g., Li Y, Hermanson D L, Moriarity B S Kaufman D S, Cell Stem Cell. 2018; 23: 181-192);
(9) scFv-NKp46TM-2B4IC-CD3ζs (see, e.g., Li Y, Hermanson D L, Moriarity B S Kaufman D S, Cell Stem Cell. 2018; 23: 181-192);
(10) scFv-NKG2DTM-2B4IC-CD3ζs (see, e.g., Li Y, Hermanson D L, Moriarity B S Kaufman D S, Cell Stem Cell. 2018; 23: 181-192);
(11) scFv-NKG2DTM-4-1BBIC-CD3ζs (see, e.g., Li Y, Hermanson D L, Moriarity B S Kaufman D S, Cell Stem Cell. 2018; 23: 181-192);
(12) scFv-NKG2DTM-2B4IC-DAP12IC-CD3ζs (see, e.g., Li Y, Hermanson D L, Moriarity B S Kaufman D S, Cell Stem Cell. 2018; 23: 181-192);
(13) scFv-NKG2DTM-2B4IC-DAP10IC-CD3ζs (see, e.g., Li Y, Hermanson D L, Moriarity B S Kaufman D S, Cell Stem Cell. 2018; 23: 181-192);
(14) scFv-NKG2DTM-4-1BBIC-2B4IC-CD3ζS (see, e.g., Li Y, Hermanson D L, Moriarity B S Kaufman D S, Cell Stem Cell. 2018; 23: 181-192); and
(15) scFv-NKG2DTM-CD3ζS (see, e.g., Li Y, Hermanson D L, Moriarity B S Kaufman D S, Cell Stem Cell. 2018; 23: 181-192).

An illustrative CAR of the disclosure is shown in FIG. 8 where the fusion protein is encoded by a lentivirus expression vector and where “SP” is a signal peptide, the CAR is an anti-FITC CAR, a CD8a hinge is present, a transmembrane domain is present (“TM”), the co-stimulation domain is 4-1BB, and the activation signaling domain is CD3ζ.

An illustrative nucleotide sequence encoding a CAR may comprise SEQ ID NO: 13:

(SEQ ID NO: 13)
ATGGCCTTACCAGTGACCGCCTTGCTCCTGCCGCTGGCCTTGCTGCTCC
ACGCCGCCAGGCCGGATGTCGTGATGACCCAGACCCCCCTCAGCCTCCC
AGTGTCCCTCGGTGACCAGGCTTCTATTAGTTGCAGATCCAGCCAGTCC
CTCGTGCACTCTAACGGTAATACCTACCTGAGATGGTATCTCCAGAAGC
CCGGACAGAGCCCTAAGGTGCTGATCTACAAAGTCTCCAACCGGGTGTC
TGAGTCCCTGACCGCTTCTCAGGGAGCGGTTCCGGCACCGACTTCACCC
TGAAGATCAACCGGGTGGAGGCCGAAGACCTCGGCGTCTATTTCTGCTC
TCAGAGTACACATGTGCCCTGGACCTTCGGCGGAGGGACCAAGCTGGAG
ATCAAAAGCTCCGCAGACGATGCCAAGAAAGATGCCGCTAAGAAAGACG
ATGCTAAGAAAGACGATGCAAAGAAAGACGGTGGCGTGAAGCTGGATGA
AACCGGAGGAGGTCTCGTCCAGCCAGGAGGAGCCATGAAGCTGAGTTGC
GTGACCAGCGGATTCACCTTTGGGCACTACTGGATGAACTGGGTGCGAC
AGTCCCCAGAGAAGGGGCTCGAATGGGTCGCTCAGTTCAGGAACAAACC
CTACAATTATGAGACATACTATTCAGACAGCGTGAAGGGCAGGTTTACT
ATCAGTAGAGACGATTCCAAATCTAGCGTGTACCTGCAGATGAACAATC
TCAGGGTCGAAGATACAGGCATCTACTATTGCACAGGGGCATCCTATGG
TATGGAGTATCTCGGTCAGGGGACAAGCGTCACAGTCAGTTTCGTGCCG
GTCTTCCTGCCAGCGAAGCCCACCACGACGCCAGCGCCGCGACCACCAA
CACCGGCGCCCACCATCGCGTCGCAGCCCCTGTCCCTGCGCCCAGAGGC
GTGCCGGCCAGCGGCGGGGGGCGCAGTGCACACGAGGGGGCTGGACTTC
GCCTGTGATATCTACATCTGGGCGCCCTTGGCCGGGACTTGTGGGGTCC
TTCTCCTGTCACTGGTTATCACCCTTTACTGCAACCACAGGAACCGTTT
CTCTGTTGTTAAACGGGGCAGAAAGAAACTCCTGTATATATTCAAACAA
CCATTTATGAGACCAGTACAAACTACTCAAGAGGAAGATGGCTGTAGCT
GCCGATTTCCAGAAGAAGAAGAAGGAGGATGTGAACTGAGAGTGAAGTT
CAGCAGGAGCGCAGACGCCCCCGCGTACCAGCAGGGCCAGAACCAGCTC
TATAACGAGCTCAATCTAGGACGAAGAGAGGAGTACGATGTTTTGGACA
AGAGACGTGGCCGGGACCCTGAGATGGGGGGAAAGCCGAGAAGGAAGAA
CCCTCAGGAAGGCCTGTACAATGAACTGCAGAAAGATAAGATGGCGGAG
GCCTACAGTGAGATTGGGATGAAAGGCGAGCGCCGGAGGGGCAAGGGGC
ACGATGGCCTTTACCAGGGTCTCAGTACAGCCACCAAGGACACCTACGA
CGCCCTTCACATGCAGGCCCTGCCCCCTCGCTAA

An illustrative CAR amino acid sequence may comprise SEQ ID NO: 14:

(SEQ ID NO: 14)
MALPVTALLLPLALLLHAARPDVVMTQTPLSLPVSLGDQASISCRSSQS
LVHSNGNTYLRWYLQKPGQSPKVLIYKVSNRVSGVPDRFSGSGSGTDFT
LKINRVEAEDLGVYFCSQSTHVPWTFGGGTKLEIKSSADDAKKDAAKKD
DAKKDDAKKDGGVKLDETGGGLVQPGGAMKLSCVTSGFTFGHYWMNWVR
QSPEKGLEWVAQFRNKPYNYETYYSDSVKGRFTISRDDSKSSVYLQMNN
LRVEDTGIYYCTGASYGMEYLGQGTSVTVSFVPVFLPAKPTTTPAPRPP
TPAPTIASQPLSLRPEACRPAAGGAVHTRGLDFACDIYIWAPLAGTCGV
LLLSLVITLYCNHRNRFSVVKRGRKKLLYIFKQPFMRPVQTTQEEDGCS
CRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKG
HDGLYQGLSTATKDTYDALHMQALPPR

An illustrative nucleotide insert may comprise SEQ ID NO: 15:

(SEQ ID NO: 15)
GCCACCATGGCCTTACCAGTGACCGCCTTGCTCCTGCCGCTGGCCTTGC
TGCTCCACGCCGCCAGGCCGGATGTCGTGATGACCCAGACCCCCCTCAG
CCTQCCAGTGTCCCTCGGTGACCAGGCTTCTATTAGTTGCAGATCCAGC
CAGTCCCTCGTGCACTCTAACGGTAATACCTACCTGAGATGGTATCTCC
AGAAGCCCGGACAGAGCCCTAAGGTGCTGATCTACAAAGTCTCCAACCG
GGTGTCTGGAGTCCCTGACCGCTTCTCAGGGAGCGGTTCCGGCACCGAC
TTCACCCTGAAGATCAACCGGGTGGAGGCCGAAGACCTCGGCGTCTATT
TCTGCTCTCAGAGTACACATGTGCCCTGGACCTTCGGCGGAGGGACCAA
GCTGGAGATCAAAAGCTCCGCAGACGATGCCAAGAAAGATGCCGCTAAG
AAAGACGATGCTAAGAAAGACGATGCAAAGAAAGACGGTGGCGTGAAGC
TGGATGAAACCGGAGGAGGTCTCGTCCAGCCAGGAGGAGCCATGAAGCT
GAGTTGCGTGACCAGCGGATTCACCTTTGGGCACTACTGGATGAACTGG
GTGCGACAGTCCCCAGAGAAGGGGCTCGAATGGGTCGCTCAGTTCAGGA
ACAAACCCTACAATTATGAGACATACTATTCAGACAGCGTGAAGGGCAG
GTTTACTATCAGTAGAGACGATTCCAAATCTAGCGTGTACCTGCAGATG
AACAATCTCAGGGTCGAAGATACAGGCATCTACTATTGCACAGGGGCAT
CCTATGGTATGGAGTATCTCGGTCAGGGGACAAGCGTCACAGTCAGTTT
CGTGCCGGTCTTCCTGCCAGCGAAGCCCACCACGACGCCAGCGCCGCGA
CCACCAACACCGGCGCCCACCATCGCGTCGCAGCCCCTGTCCCTGCGCC
CAGAGGCGTGCCGGCCAGCGGCGGGGGGCGCAGTGCACACGAGGGGGCT
GGACTTCGCCTGTGATATCTACATCTGGGCGCCCTTGGCCGGGACTTGT
GGGGTCCTTCTCCTGTCACTGGTTATCACCCTTTACTGCAACCACAGGA
ACCGTTTCTCTGTTGTTAAACGGGGCAGAAAGAAACTCCTGTATATATT
CAAACAACCATTTATGAGACCAGTACAAACTACTCAAGAGGAAGATGGC
TGTAGCTGCCGATTTCCAGAAGAAGAAGAAGGAGGATGTGAACTGAGAG
TGAAGTTCAGCAGGAGCGCAGACGCCCCCGCGTACCAGCAGGGCCAGAA
CCAGCTCTATAACGAGCTCAATCTAGGACGAAGAGAGGAGTACGATGTT
TTGGACAAGAGACGTGGCCGGGACCCTGAGATGGGGGGAAAGCCGAGAA
GGAAGAACCCTCAGGAAGGCCTGTACAATGAACTGCAGAAAGATAAGAT
GGCGGAGGCCTACAGTGAGATTGGGATGAAAGGCGAGCGCCGGAGGGGC
AAGGGGCACGATGGCCTTTACCAGGGTCTCAGTACAGCCACCAAGGACA
CCTACGACGCCCTTCACATGCAGGCCCTGCCCCCTCGCTAA

In various embodiments, CAR-expressing cells comprising the nucleic acid of SEQ ID NO: 13 or 15 are provided. In some embodiments, a chimeric antigen receptor polypeptide comprising SEQ ID NO: 14 is contemplated. In some embodiments, a vector is contemplated comprising SEQ ID NO: 13 or 15. In some embodiments, a lentiviral vector is contemplated comprising SEQ ID NO: 13 or 15. In some embodiments, SEQ ID NO: 14 can comprise or consist of human or humanized amino acid sequences.

In some embodiments, variant nucleic acid sequences or amino acid sequences having at least about 80%, at least about 90%, at least about 95%, at least about 97%, at least about 98%, at least about 99%, or at least about 99.5% sequence identity to SEQ ID NO: 13, SEQ ID NO: 14, or SEQ ID NO: 15 are contemplated.

While the affinity at which the CARs, expressed by the lymphocytes, bind to the targeted moiety can vary, and in some cases low affinity binding may be preferable (such as about 50 nM), the binding affinity of the CARs to the targeted ligand will generally be at least about 100 nM, 1 pM, or 10 pM, preferably at least about 100 pM, 1 fM or 10 fM, even more preferably at least about 100 fM.

### Therapeutic Compositions

In some embodiments, the present disclosure provides a composition comprising one or more cell populations.

In some embodiments, the composition comprises a population of differentiated NK cells. In some embodiments, the composition comprises a population of differentiated NK cells that is at least 40%, at least 50%, at least 60%, at least 70%, at least 80%, at least 90% or 100% of CD34+CD43+CD45+LFA1+ quadruple-positive cells.

In some embodiments, the composition comprises a population of differentiated NK cells that is about 40% to 50%, about 50% to 60%, about 60% to 70%, about 70% to 80%, about 80% to 90%, or about 90% to 100% of CD34+CD43+CD45+LFA1+ quadruple-positive cells.

In some embodiments, the composition comprises a population of mature NK cells. In some embodiments, the composition comprises a population of mature NK cells that is at least 40%, at least 50%, at least 60%, at least 70%, at least 80%, at least 90% or 100% of CD34+CD43+CD45+LFA1+ NKp46+NKG2D+LFA1+CD161−CD73− cells.

In some embodiments, the composition comprises a population of mature NK cells that is about 40% to 50%, about 50% to 60%, about 60% to 70%, about 70% to 80%, about 80% to 90%, or about 90% to 100% of CD34+CD43+CD45+LFA1+ NKp46+NKG2D+LFA1+CD161−CD73− cells.

### Methods of Treating

The present disclosure provides methods of treating a subject in need thereof with the compositions, therapeutic compositions, or cells, disclosed herein. In some embodiments, the disclosure provides a method of treating cancer and/or killing cancer cells in a subject, comprising administering a therapeutically effective amount of the disclosed cells to the subject.

In some embodiments, the cancer is a solid tumor, sarcoma, carcinoma, lymphoma, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), chronic or acute leukemia, acute myeloid leukemia, chronic myeloid leukemia, acute lymphoblastic leukemia (ALL) (including non T-cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma)), monoclonal gammapathy of undetermined significance (MGUS), plasmacytomas (e.g., plasma cell dyscrasia, solitary myeloma, solitary plasmacytoma, extramedullary plasmacytoma, and multiple plasmacytoma), systemic amyloid light chain amyloidosis, POEMS syndrome (also known as Crow-Fukase syndrome, Takatsuki disease, and PEP syndrome), or a combination thereof.

In some embodiments, a method disclosed herein may be used to treat cancer and/or kill cancer cells in a subject by administering a therapeutically effective amount of the cells according to any of the foregoing embodiments.

The present disclosure also provides a method of treating cancer and/or killing cancer cells in a subject, comprising administering the composition of any of the foregoing embodiments to the subject.

In some embodiments, the present disclosure provides a method of treating cancer with any of the compositions provided herein. “Cancer” has its plain and ordinary meaning when read in light of the specification, and may include but is not limited to, for example, a group of diseases involving abnormal cell growth with the potential to invade or spread to other parts of the body. Subjects that can be addressed using the methods described herein include subjects identified or selected as having cancer, including but not limited to colon, lung, liver, breast, renal, prostate, ovarian, skin (including melanoma), bone, and brain cancer, etc. Such identification and/or selection can be made by clinical or diagnostic evaluation. In some embodiments, the tumor associated antigens or molecules are known, such as melanoma, breast cancer, brain cancer, squamous cell carcinoma, colon cancer, leukemia, myeloma, and/or prostate cancer. Examples include but are not limited to B cell lymphoma, breast cancer, brain cancer, prostate cancer, and/or leukemia. In some embodiments, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia. In some embodiments, a method of treating, ameliorating, or inhibiting a cancer in a subject is provided. In some embodiments, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer.

In some embodiments, an additional cancer therapy is provided, such as a small molecule, e.g., a chemical compound, an antibody therapy, e.g., a humanized monoclonal antibody with or without conjugation to a radionuclide, toxin, or drug, surgery, and/or radiation.

In some embodiments, the subject is selected to receive an additional cancer therapy, which can include a cancer therapeutic, radiation, chemotherapy, or a drug for the treatment of cancer. In some embodiments, the drugs comprise Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T, Sorafenib, Sunitinib, Talc Intrapleural, Tamoxifen, Temozolomide, Temsirolimus, Thalidomide, Trastuzumab, Vinorelbine or Zoledronic acid.

### Modes of Administration and Dosing

In some embodiments, NK cells may be grown in conditions that are suitable for a population of cells that will be introduced into a subject such as a human. Specific considerations include the use of culture media that lacks any animal products, such as bovine serum. Other considerations include sterilized-condition to avoid contamination of bacteria, fungi and mycoplasma.

In some embodiments, after transfection, the cells can be immediately administered to the patient or the cells can be cultured for at least about 1, 2, 3, 4, 5, 6, 7, 8, 9, 10, 11, 12, 13, 14, 15, 16, 17, 18 or more days, or about 5 and about 12 days, about 6 and about 13 days, about 7 and about 14 days, or about 8 and about 15 days, for example, to allow time for the cells to recover from the transfection. Suitable culture conditions can be similar to the conditions under which the cells were cultured for activation either with or without the agent that was used to promote activation.

The disclosed cells may be administered in a number of ways depending upon whether local or systemic treatment is desired.

In the case of adoptive cell therapy, methods for administration of cells for adoptive cell therapy are known and may be used in connection with the provided methods and compositions.

In general, administration may be topical, parenteral, or enteral. The compositions of the disclosure are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, intrasynovial injection or infusions; and kidney dialytic infusion techniques. In an embodiment, parenteral administration of the compositions of the present disclosure comprises intravenous administration.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Illustrative parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The present compositions of viral particles, adaptor molecules, and/or immune cells may be administered in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of infusing differentiated cells or transgenic differentiated cells according to the disclosure, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 10³ to about 10⁹ cells per kg body weight including all integer values of cell numbers within those ranges.

### Kits

Also provided herein are kits, including the cells described herein, as well as written instructions for making and using the same. Thus, for example, provided herein is a kit including the cell population as described herein written instructions for making and using the same.

In some embodiments, a kit can have one or more additional therapeutic agents that can be administered simultaneously or in sequence with another component for a desired purpose, e.g., genome edition or cell therapy.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (such as associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### EXAMPLES

### Example 1: Development of Xenogenic-Free and Serum-Free Suspension Culture NK Cell Differentiation Method

The purpose of this study was to develop a three-dimensional (3D) serum-free and xenogenic-free method for differentiating NK cells from stem cells. Specifically, undifferentiated human iPSCs were maintained in mTeSR Plus media (STEMCELL Technologies) on hESC-qualified Matrigel (Corning) and routinely passaged using EDTA (Thermo Fisher Scientific). For pluripotent suspension culture, undifferentiated human iPSCs were passaged with Gentle Cell Dissociation Reagent (GCDR; STEMCELL Technologies) to generate small aggregates and aggregates were seeded into one well of a 6-well plate (non-tissue culture-treated) at a concentration of 5×10⁵ cells/mL at 2 mL/well in mTeSR 3D (serum-free; STEMCELL Technologies) and cultured on an orbital shaker (1″ diameter throw, set to 70 rpm). Cells continued culture on the orbital shaker (70 rpm) for the entire differentiation process. iPSCs were batch-fed following manufacturer's instructions, adding media each day for 4 days, before passaging following manufacturer's instructions, using a 37 um filter to remove single cells and GCDR and additional filtering to dissociate aggregates into smaller sizes for re-seeding. Pluripotent aggregates were thus cultured and passaged for 4-8 days before initiating differentiation. Pluripotent aggregates were differentiated into iNKs in suspension using media from the STEMdiff NK Cell Kit (STEMCELL Technologies).

Briefly, at day 0, uniform pluripotent aggregates generated using the suspension passaging protocol described above were seeded into each well on a 6-well plate (non-tissue culture-treated) at 10,000 clumps per well and 2 mL/well in STEMdiff Hematopoietic—EB Basal Medium supplemented with EB Supplement A (serum-free) to generate embryoid bodies (EBs). Suspended EBs were cultured in STEMdiff Hematopoietic—EB Basal Medium supplemented with EB Supplement A (days 0-3) and then EB Supplement B (days 3-12) (serum-free), with partial media changes performed. At day 12, EBs were harvested and dissociated into a single-cell suspension and re-seeded (no CD34+ selection process was performed) onto a non-tissue culture-treated 6-well plate coated with StemSpan Lymphoid Progenitor Differentiation Coating, at a density of 50,000 cells per well, in 2.5 mL/well, with StemSpan SFEM II media supplemented with StemSpan Lymphoid Progenitor Expansion Supplement (days 12-26), with partial media changes performed. On day 19, cells were transferred to a newly coated plate. On day 26, cells were reseeded onto 6-well non-treated, non-coated plates (250,000 cells per well, 2.5 mL/well) with StemSpan SFEM II media supplemented with StemSpan NK Cell Differentiation Supplement and UM729 (1 uM)(days 26-40), with partial media changes. At day 40, NK cells were harvested. FIG. 1 provides a schematic of the differentiation method #1.

### Example 2: Development of a 3D Xenogenic-Free and Serum-Free HP Differentiation Method

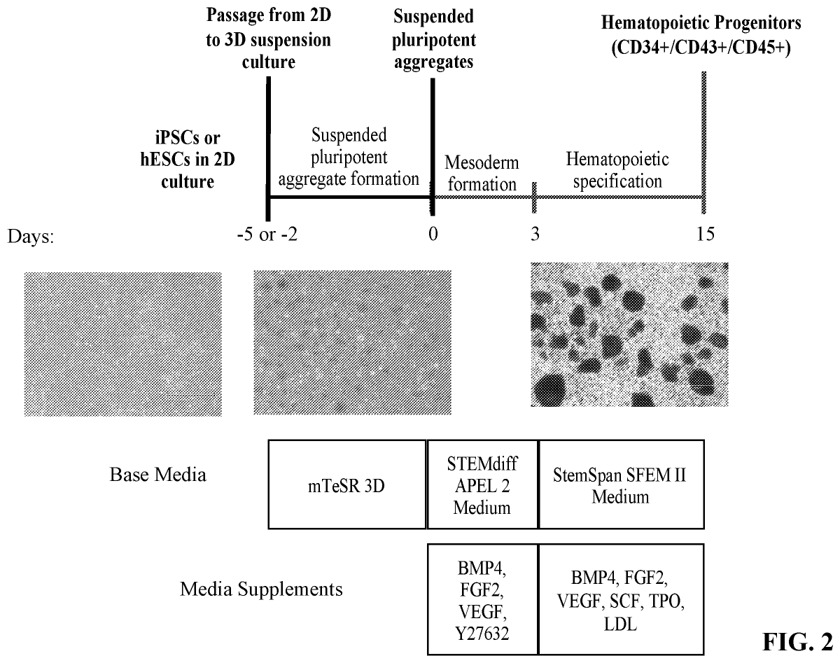

The purpose of this study was to develop a 3D serum-free and xenogenic-free method of differentiating hematopoietic progenitors (HPs) from stem cells. Such HPs can be further differentiated into immune cell populations such as NK cells. Undifferentiated human iPSCs were maintained and aggregates were formed as described in Example 1. At day 0, uniform aggregates were generated using the suspension passaging protocol described above, and 800 clumps were seeded into each well on a 6-well plate (non-tissue culture-treated) at 2 mL/well in STEMdiff APEL 2 Medium (STEMCELL Technologies; media is fully defined, serum- and animal component-free) supplemented with BMP4 (5 to 50 ng/mL), FGF2 (5 to 50 ng/mL), VEGF (5 to 100 ng/mL), and Y27632 (1-20 μM)(days 0-3) to generate embryoid bodies (EBs) in suspension. To induce hematopoietic progenitor formation, EBs were cultured in StemSpan SFEM II Medium (STEMCELL Technologies; serum-free) supplemented with combinations of BMP4 and FGF2 (5 to 50 ng/mL), VEGF and SCF (up to 100 ng/mL), TPO (up to 100 ng/mL) and LDL (up to 50 μg/mL) (days 3-15). FIG. 2 provides a schematic of the differentiation method #2.

### Example 3: Characterization of Hematopoietic Progenitors (HPs)

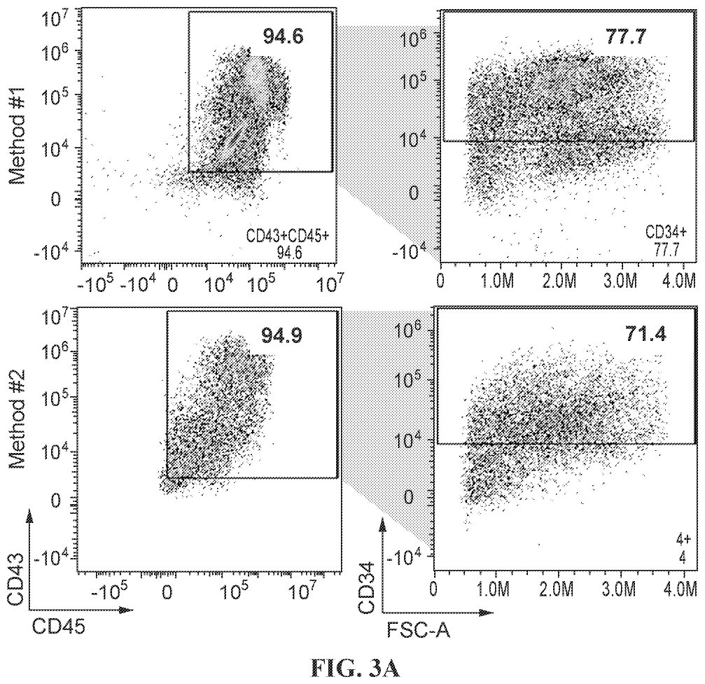

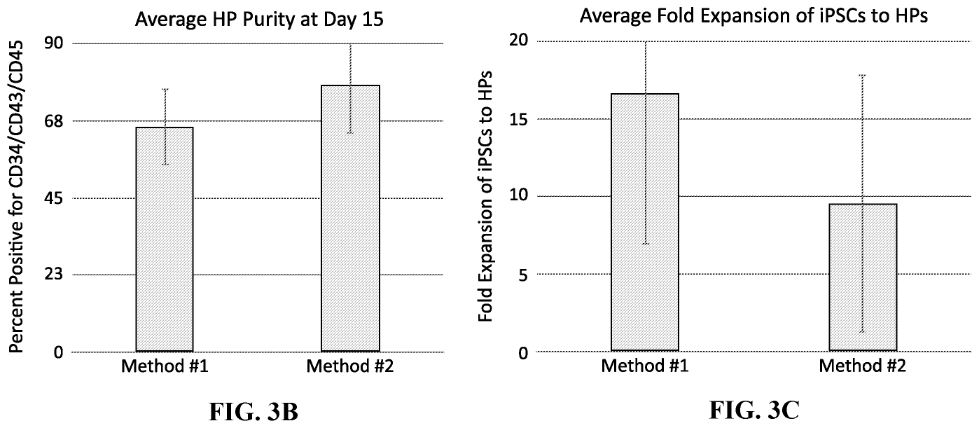

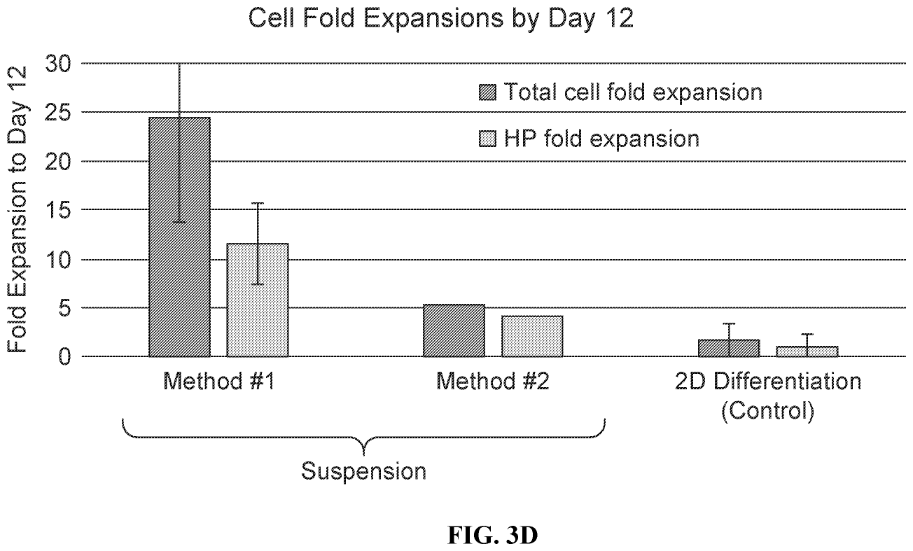

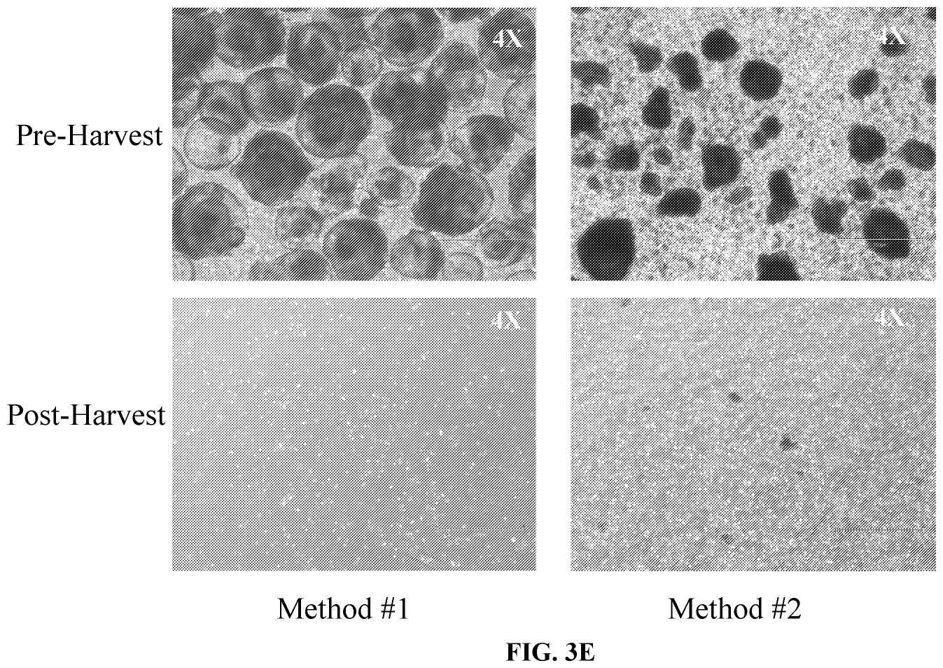

HPs were induced to form from iPSC-derived EBs as described in Examples 1 and 2. To determine the percentage of HPs generated of the total cells present at days 12-15 of differentiation, flow cytometry analysis was performed, gating cells to quantify percentage of cells triple-positive for the HP markers CD34/CD43/CD45 (FIG. 3A).

High HP purity was observed for both methods, ranging from 58-74% for Method #1 and 68-88% for Method #2 of all cells being triple-positive for CD34/CD43/CD45 (FIG. 3B). High yields of HPs were observed, ranging from 9.8 to 23.6-fold expansion at day 12 for Method #1 and 3.6 to 15.4-fold expansions at day 15 for Method #2 of HPs relative to iPSCs seeded at day 0 (FIG. 3C). A comparison between the suspension protocols to a standard 2D differentiation protocol is shown in FIG. 3D. Representative brightfield microscope images are shown of the EBs prior to HP harvesting, and post HP harvesting, at day 12-15 (FIG. 3E). This experiment shows that Method #1 and Method #2 resulted in a high yield of HPs after 15 days of differentiation based on CD34+/CD43+/CD45+ marker expression (>65%) and high fold of iPSC-to-HP expansion.

### Example 4: Characterization of iNK Cells

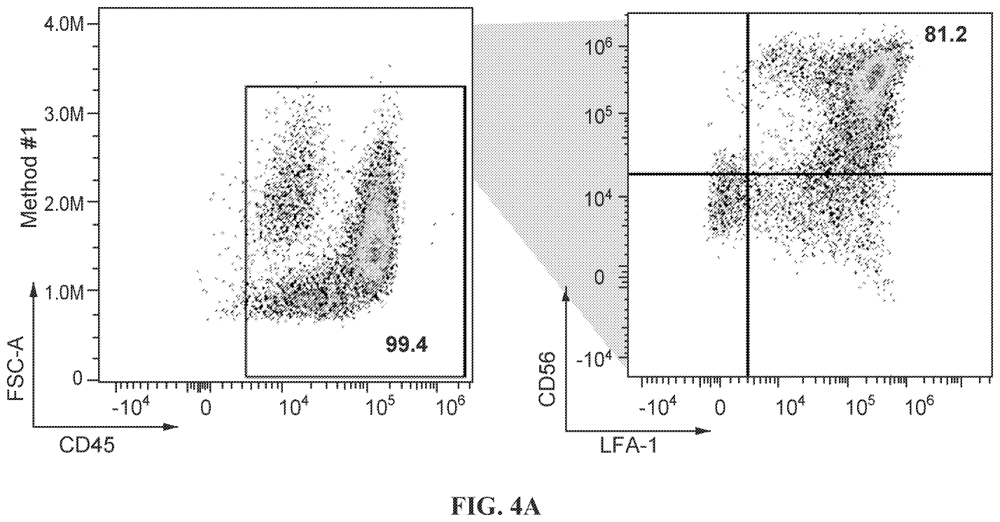

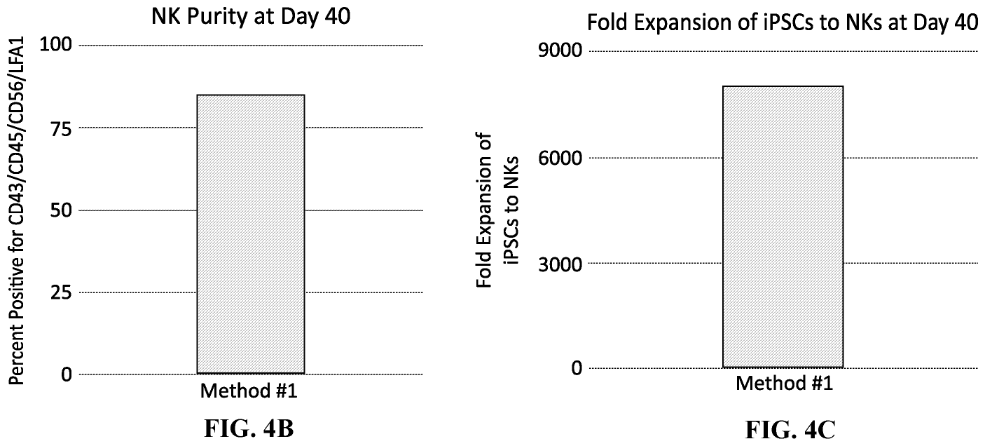

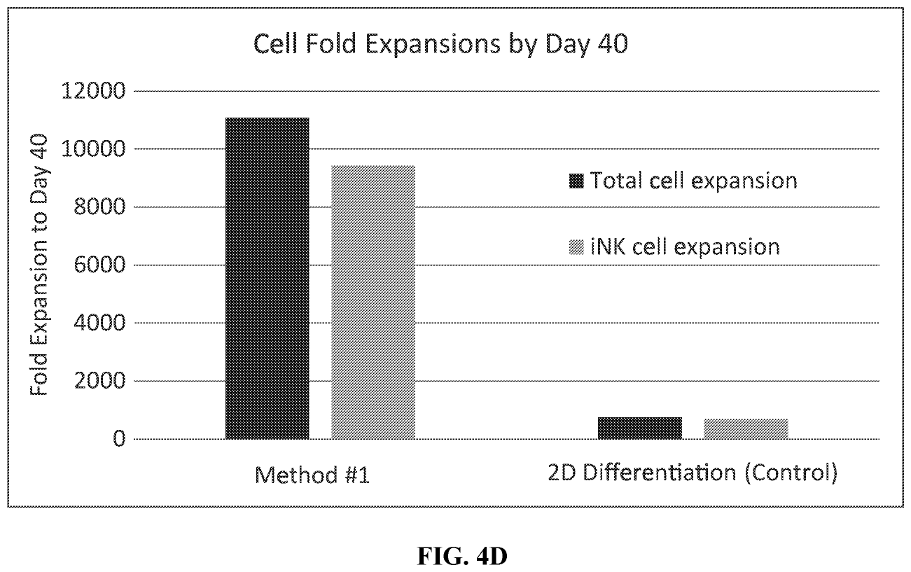

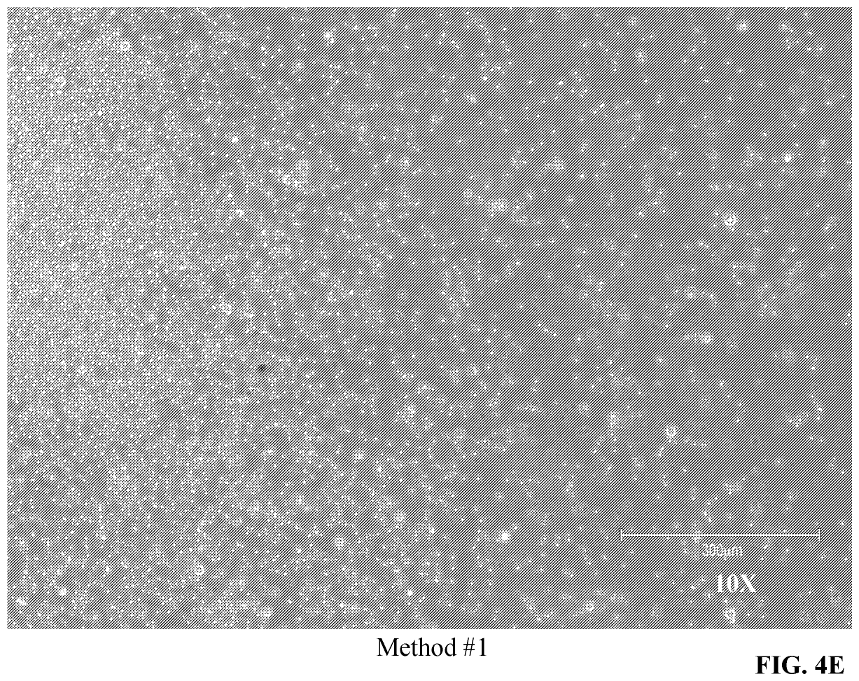

iNKs were induced to form from iPSC-derived HPs as described in Example 1. To determine the percentage of iNKs generated of the total cells present at day 40 of differentiation, flow cytometry analysis was performed, gating cells to quantify percentage of cells positive for three NK markers, CD45/CD56/LFA1 (FIG. 4A). High-purity iNKs were observed, with 80.6% of all cells being triple-positive for NK markers CD45/CD56/LFA1 (FIG. 4B). High yields of iNKs were also observed, at 8,024-fold expansion of iNKs at day 40 relative to iPSCs seeded at day 0 (FIG. 4C). A comparison between the suspension protocol to a standard 2D differentiation protocol is shown in FIG. 4D. Representative brightfield microscope image shown of the cells at day 40 of iNK differentiation (FIG. 4E). This experiment shows that Method #1 resulted in high purity and yield of NKs after 40 days of differentiation based on CD45+/CD56+/LFA1+ marker expression.

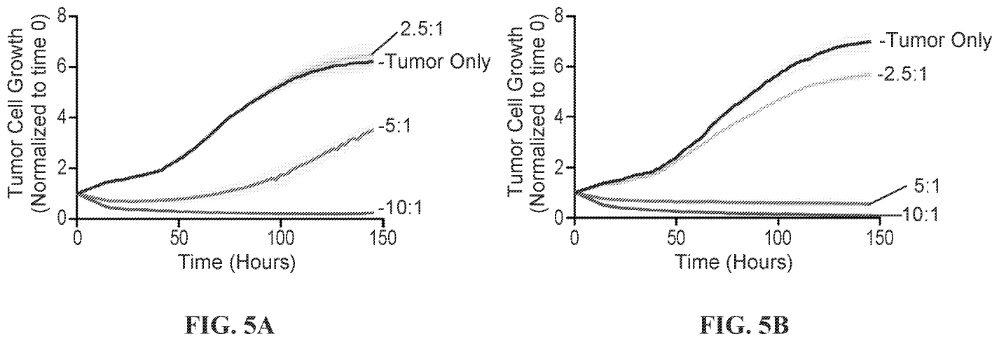

Next, the cytotoxicity of the iNK cells was determined. Day 40 iNKs harvested from Method #1 (shown in FIGS. 4A-D) were incubated with breast adenocarcinoma MDA-MB231 cells expressing a nuclear fluorescent protein at different E:T ratios (2.5:1, 5:1, 10:1) in the absence (unstimulated, FIG. 5A) or presence (stimulated, FIG. 5B) of cytokines IL-2 and IL-15. Cell mixtures were placed into an IncuCyte fluorescent microscope and imaged every 2 hours. MDA cells were quantified over time via fluorescent marker and graphed as the ratio of MDA cells compared to time 0 (time 0 is equal to one). iNK cells reduced MDA growth in a dose-responsive manner, with faster clearance with cytokine support. This experiment shows that Method #1 resulted in producing functionally active cells capable of reducing tumor cell growth.

### Example 5: Development of a 3D Xenogenic-Free and Serum-Free HP Differentiation Method with Engineered Cells

To increase the proliferation of the cell populations from Examples 1-4, iPSCs were genetically engineered using methods well known in the art, such as in Ran et al. Nature Protocols, Vol. 8, pgs. 2281-2308 (2013), Liu et al. The CRISPR Journal, Vol. 3, Issue 3 (2020), and General CRISPR RNP Transfection Guidelines by Thermo Fisher Scientific, which are incorporated by reference in their entirety. In this example, electroporation was used. Generally, iPSCs were engineered to modulate expression and knockout various endogenous loci as described using CRISPR-Cas9-mediated gene editing. The cells were electroporated with ribonucleoprotein (RNP) complexes comprising a Cas9 and a guide RNA that targeted the specific locus indicated.

CRISPR-Cas9 is used to generate site-specific knock-ins of constructs encoding a rapamycin activated cytokine receptor (RACR) via homology-directed repair (HDR) at various loci. RACR is a synthetic cytokine receptor activated by the small molecule rapamycin, or rapalogs, and drives cell proliferation.

RACR was first knocked into the B2M locus with simultaneous B2M knock out and is expressed using a using a dual promoter system containing both the endogenous B2M promoter as well as an exogenously provided EF1a promoter. The endogenous B2M promoter alone is insufficient to produce high levels of RACR. This dual promoter system resulted in RACR levels that are equivalent or higher than what can be reached through lentiviral transduction of RACR with a strong exogenous MND promoter. Removing B2M through gene knock out also reduces allogeneic anti-graft responses of cell therapies by removing CD8 T cell mis-match responses.

RACR requires rapamycin for activation; however, rapamycin inhibits cell growth. To generate rapamycin resistant cells, the FKBP12 gene was knocked-out. In wildtype cells, FKBP12 binds to rapamycin creating a novel binding surface that enables FKBP12-rapamycin to dimerize with the FRB domain of mTOR and subsequently inhibit mTOR. In the absence of FKBP12, rapamycin cannot interact with the mTOR complex and thus loses its inhibitory capacity.

After obtaining the engineered FKBP12 knockout iPSCs that encode RACR (B2M-EF1a-RACR and FKBP12 KO cells), the cells were seeded at a concentration of 8×10⁴ cells/well. Next, the cells were treated with a dissociation reagent, followed by adaption to 3D suspension culture with agitation or will be directly passed into 3D suspension differentiation method without any adaption or agitation.

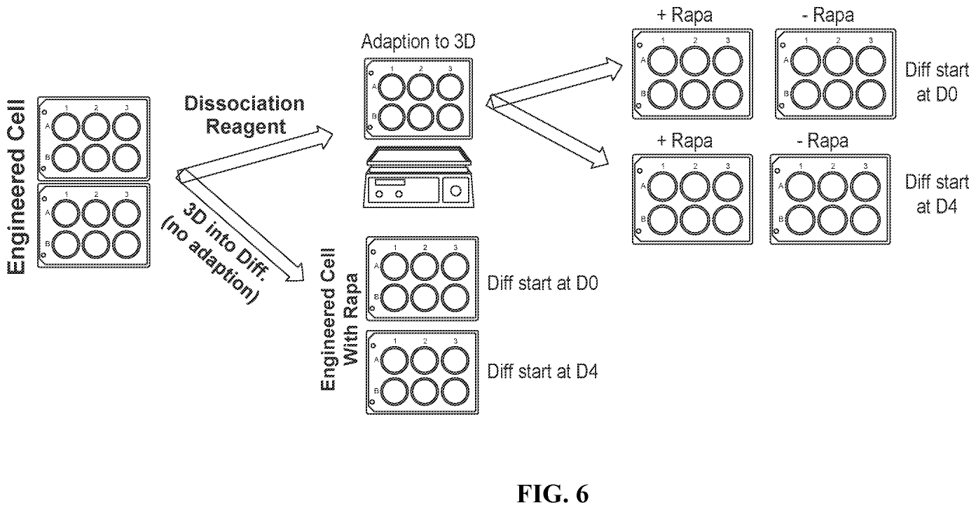

Next, the cells were treated with rapamycin followed by changing to differentiation media at either Day 0 or Day 4 that contains high FGF (100 ng/ml vs 10 ng/ml). As a control, cells were not treated with rapamycin. A 50% medium change occurs on Day 2 of differentiation. FIG. 6 provides a schematic of the differentiation method that uses engineered cells that express RACR.

The cells were differentiated into HP cells following the protocols described herein. HP cells were characterized as detailed in Example 3, respectively.

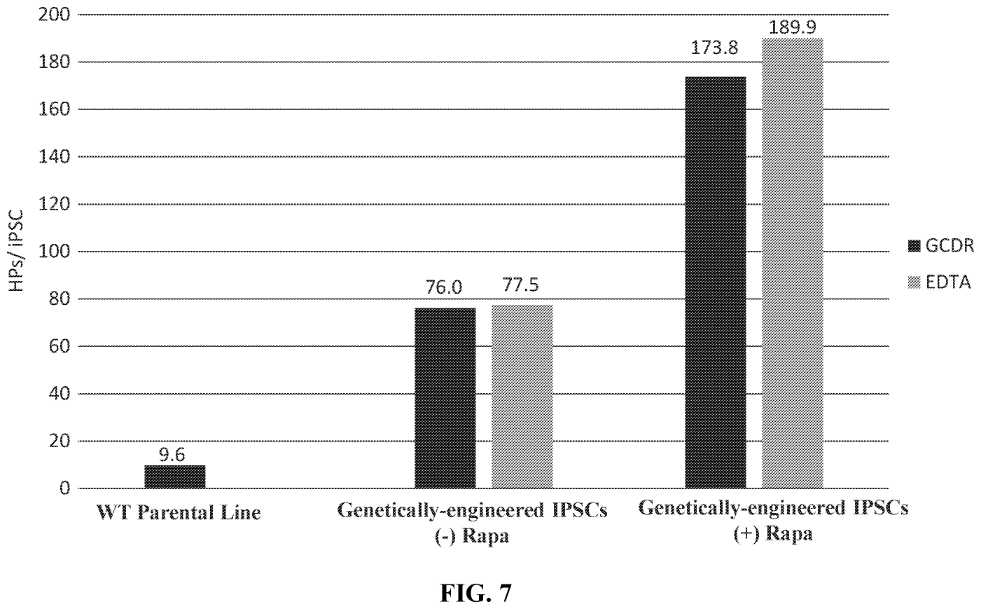

To determine if engineered cells produce more cells compared to non-engineered cells, the expansion of iPSC cells to HP was measured after 14 days in 3D suspension culture. Cells were adapted to 3D culture (for 1 passage and kept in the stem cell undifferentiated state) then passed to a second 3D culture where differentiation to iNKs was started (either at Day 0 or the same day as the passage). Analysis was performed 14 days into the differentiation process to determine HP generation. The analysis is summarized in Table 1. FIG. 7 shows the HP:iPSC ratio after 14 days for either parental cells or engineered cells treated with or without rapamycin and treatment. No differences were observed using either EDTA or GDCR to passage the cells from 2D culture to 3D culture. This experiment shows that the engineered cells produce more HPs than the parental cell line.

TABLE 1
Summary of 3D Suspension Culture for Engineered Cells

| | | | | | % | | | | |
|---|---|---|---|---|---|---|---|---|---|
| | # | | Viable | Total | CD43+ | % CD | % | Absolute | HP/ |
| Sample | Cells | Viability | Cells/ml | Cells | CD45+ | 34+ | HP | HP | iPSC |
| Parental | 8 × 10⁴ | 83.68 | 1.94 × 10⁶ | 2.13 × 10⁶ | 98.00 | 36.80 | 36.06 | 7.70 × 10⁵ | 9.62 |
| Line | | | | | | | | | |
| Engineered | 8 × 10⁴ | 75.15 | 3.01 × 10⁶ | 6.62 × 10⁶ | 94.80 | 96.90 | 91.86 | 6.08 × 10⁶ | 76.04 |
| Cell GCDR | | | | | | | | | |
| (−) Rapa | | | | | | | | | |
| Engineered | 8 × 10⁴ | 63.44 | 6.62 × 10⁶ | 1.46 × 10⁷ | 96.80 | 98.60 | 95.44 | 1.39 × 10⁷ | 173.76 |
| Cell GCDR | | | | | | | | | |
| (+) Rapa | | | | | | | | | |
| Engineered | 8 × 10⁴ | 67.93 | 3.58 × 10⁶ | 7.52 × 10⁶ | 91.50 | 90.10 | 82.44 | 6.20 × 10⁶ | 77.47 |
| Cell EDTA | | | | | | | | | |
| (−) Rapa | | | | | | | | | |
| Engineered | 8 × 10⁴ | 56.03 | 6.6 × 10⁶ | 1.58 × 10⁷ | 96.60 | 99.30 | 95.92 | 1.52 × 10⁷ | 189.93 |
| Cell EDTA | | | | | | | | | |
| (+) Rapa | | | | | | | | | |

1. A method for generating a population of CD34+/CD43+/CD45+ cells, comprising:
(i) culturing a population of progenitor cells in a two-dimensional (2D) culture system for a period of time sufficient to form progenitor cell aggregates;
(ii) passaging the progenitor cell aggregates from the 2D culture system to a three-dimensional (3D) suspension culture system;
(iii) contacting the progenitor cell aggregates in the 3D suspension culture system with a differentiation media for a period of time sufficient to generate the population of CD34+/CD43+/CD45+ cells.

2. A method for differentiating a population of stem cells into a population of hematopoietic progenitors, comprising:
(i) culturing the population of stem cells in a 2D culture system for a period of time sufficient to form stem cell aggregates;
(ii) passaging the stem cell aggregates from the 2D culture system to a 3D suspension culture system;
(iii) contacting the stem cell aggregates in the 3D suspension culture system with a differentiation media comprising a bone morphogenetic protein (BMP) pathway activator, a fibroblast growth factor (FGF), and a vascular endothelial growth factor (VEGF), for a period of time sufficient to differentiate the population of stem cells into the population of hematopoietic progenitors.

3. The method of claim 1 or 2, wherein the 3D suspension culture has a volume of between 50-50,000 ml.

4. The method of any one of claims 1 to 3, wherein the 3D suspension culture is agitated.

5. The method of claim 3, wherein the 3D suspension culture is agitated at a rate of between 10 revolutions per minute (RPM) to 100 RPM.

6. The method of claim 4 or 5, wherein the 3D suspension culture is agitated at a rate of 70 RPM.

7. The method of any one of claims 2 to 6, wherein the population of hematopoietic progenitors comprises CD34+/CD43+/CD45+ cells.

8. The method of any one of claims 2-7, wherein the BMP pathway activator is BMP4.

9. The method of any one of claims 2-8, wherein the FGF is FGF2.

10. The method of any one of claims 2-9, wherein the VEGF is VEGF-165.

11. The method of any one of claims 2 to 10, wherein the differentiation media comprises Rho-associated coiled coil forming protein serine/threonine kinase (ROCK) inhibitor.

12. The method of claim 11, wherein the ROCK inhibitor is Y27632.

13. The method of any one of claims 2 to 12, wherein the differentiation media comprises stem cell factor (SCF).

14. The method of any one of claims 2 to 13, wherein the differentiation media comprises thrombopoietin (TPO).

15. The method of any one of claims 2 to 14, wherein the differentiation media comprises a low-density lipoprotein (LDL).

16. The method of any one of claims 2 to 15, wherein the differentiation media comprises the BMP pathway activator, the FGF, the VEGF, and the ROCK inhibitor.

17. The method of any one of claims 2 to 16, wherein the differentiation media comprises the BMP pathway activator, the FGF, the VEGF, SCF, TPO, and the LDL.

18. The method of any one of claims 2 to 17, wherein (iii) comprises contacting the population of stem cell aggregates with the differentiation media for 1-5 days, wherein the differentiation media comprises the BMP pathway activator, the FGF, the VEGF, and optionally the ROCK inhibitor.

19. The method of any one of claims 2 to 18, wherein (iii) comprises (a) contacting the stem cell aggregates for 1-5 days with the differentiation media comprising the BMP pathway activator, the FGF, the VEGF the ROCK inhibitor, to generate embryoid bodies or mesoderm cells, and (b) contacting the embryoid bodies or mesoderm cells for 1-15 days with a differentiation media comprising the BMP pathway activator, the FGF, the VEGF, SCF, TPO, and the LDL.

20. The method of claim 19, wherein the differentiation media comprises 1-50 ng/mL BMP, 1-50 ng/mL FGF, 5-100 ng/mL VEGF, 0.1-20 uM ROCK inhibitor, 1-200 ng/mL SCF, 1-100 ng/mL TPO, and 1-50 ug/mL LDL, or any combination thereof.

21. The method of any one of claims 1 to 20, wherein the progenitor cells or stem cells are induced pluripotent stem cells (iPSCs).

22. The method of any one of claims 1 to 21, wherein the progenitor cells or stem cells are human embryonic stem cells (hESCs).

23. The method of any one of claims 2 to 22, wherein the differentiation media is serum free.

24. The method of any one of claims 2 to 23, wherein the method is xenogenic-free.

25. A method of generating a population of NK cells, comprising:
(a) culturing a population of stem cells in a 2D culture system for a period of time sufficient to form stem cell aggregates;
(b) passaging the stem cell aggregates from the 2D culture system to a 3D suspension culture system;
(c) contacting the stem cell aggregates in the 3D suspension culture system with a first media comprising a BMP pathway activator, an FGF, a VEGF, and optionally an inhibitor of ROCK, for a period of time sufficient to generate embryoid bodies;
(d) contacting the embryoid bodies with a first differentiation media comprising a BMP pathway activator, a FGF, VEGF, SCF, TPO, and an LDL, for a period of time sufficient to generate a population of hematopoietic progenitors;
(e) contacting the population of hematopoietic progenitors with a second differentiation media, for a period of time sufficient to generate the population of NK cells.

26. The method of claim 25, wherein the second differentiation media comprising SCF, IL-7, IL-12, IL-15, FLT3L, a pyrimido-[4,5-b]-indole derivative, and an AhR inhibitor.

27. The method of any one of claims 25-26, wherein the media comprises 1-100 ng/mL SCF, 1-50 ng/mL IL-7, 1-100 ng/mL IL-12, 1-100 ng/mL IL-15, 1-100 ng/mL FLT3L, 0.1-10 uM pyrimido-[4,5-b]-indole derivative, 0.1-10 uM AhR antagonist, and any combination thereof.

28. The method of claim 26 or 27, wherein the pyrimido-[4,5-b]-indole derivative is UM729 and the AhR inhibitor is SR1.

29. The method of any one of claims 25-28, wherein the BMP pathway activator is BMP4, the FGF is FGF2, the VEGF is VEGF-165, and the inhibitor of ROCK is Y27632.

30. The method of any one of claims 25 to 29, wherein each media of steps (b)-(e) is serum free.

31. The method of any one of claims 25 to 30, wherein the method is xenogenic-free.

32. The method of any one of claims 25 to 31, where the first media, the first differentiation media, and the second differentiation media each comprise the same base media.

33. The method of any one of claims 25 to 31, where the first media, the first differentiation media, and the second differentiation media each comprise different base media.

34. The method of any one of claims 25 to 31, where the first differentiation media and the second differentiation media each comprise the same base media, and the first media comprises a base media different from the first and second differentiation media.

35. The method of any one of claims 25 to 31, wherein the first differentiation media and the second differentiation media each comprise a base media comprising Iscove's modified dulbecco's medium, bovine serum albumin, recombinant human insulin, human transferrin, and 2-mercaptoethanol.

36. The method of any one of claims 25 to 35, wherein the period of time of step (b) is 2-8 days, the period of time of step (c) is 1-5 days, the period of time of step (d) is 3-15 days, and the period of time of step (e) is 11-25 days.

37. The method of any one of claims 25 to 36, wherein steps (a)-(e) occur within 40-50 days.

38. The method of any one of claims 25 to 37, wherein the stem cells are induced pluripotent stem cells (iPSCs) or human embryonic stem cells (hESCs).

39. The method of any one of claims 25 to 38, wherein the population of hematopoietic progenitors comprises about 50% to about 100% CD34+/CD43+/CD45+ cells.

40. The method of any one of claims 25 to 38, wherein the population of NK cells comprises about 60% to about 100% CD43+/CD45+/CD56+/LFA1+ cells.

41. The method of any one of claims 25 to 40, comprising expanding the population of NK cells, wherein the population of NK cells expands about 1,000 to about 10,000 fold.

42. The method of any one of claims 2-41, wherein the population of stem cells is genetically engineered or edited.

43. The method of any one of claims 25 to 42, wherein the population of NK cells is genetically engineered or edited.

44. A population of cells comprising hematopoietic progenitors produced by the method of any one of claims 2-24.

45. The population of cells of claim 44, wherein the hematopoietic progenitors are CD34+/CD43+/CD45+.

46. The population of cells of claim 44 or 45, comprising 30-50% hematopoietic progenitors.

47. A population of cells comprising NK cells produced by the method of any one of claims 25-43.

48. The population of cells of claim 47, wherein the NK cells are CD45+/CD56+/LFA1+.

49. The population of cells of claim 47 or 48, comprising 60-100% NK cells.

50. A pharmaceutical composition comprising the cell population of any one of claims 44 to 49.

51. A method of generating a population of hematopoietic progenitors, comprising:
(a) genetically engineering a population of stem cells to express a synthetic cytokine receptor for a non-physiological ligand,
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain;
(b) culturing the population of stem cells in a 2D culture system for a period of time sufficient to form stem cell aggregates;
(c) passaging the stem cell aggregates from the 2D culture system to a 3D suspension culture system; and
(d) contacting the stem cell aggregates in the 3D suspension culture system with a differentiation media for a period of time sufficient to generate hematopoietic progenitors.

52. A method of generating a population of NK cells, comprising:
(a) genetically engineering a population of stem cells to express a synthetic cytokine receptor for a non-physiological ligand,
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain;
(b) culturing the population of stem cells in a 2D culture system for a period of time sufficient to form stem cell aggregates;
(c) passaging the stem cell aggregates from the 2D culture system to a 3D suspension culture system;
(d) contacting the stem cell aggregates in the 3D suspension culture system with a first differentiation media for a period of time sufficient to generate hematopoietic progenitors; and
(e) contacting the population of hematopoietic progenitors with a second differentiation media, for a period of time sufficient to generate the population of NK cells.

53. The method of claim 51 or 52, wherein the intracellular domain of the synthetic beta chain polypeptide is selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

54. The method of any one of claims 51 to 53, wherein the nucleotide sequence is inserted via homology directed repair (HDR).

55. The method of any one of claims 51 to 54, wherein the vector comprises a nucleic acid comprising from 5′ to 3′ (a) a nucleotide sequence homologous with a region located upstream of the target site, (b) the nucleotide sequence encoding a synthetic cytokine receptor for a non-physiological ligand, and (c) a nucleotide sequence homologous with a region located downstream, wherein a double-strand break occurs at the target site in the endogenous gene, and the nucleic acid is exchanged with a homologous nucleotide sequence of the endogenous gene.

56. The method of any one of claims 51 to 55, wherein the nucleotide sequence is inserted via non-homologous end joining (NHEJ).

57. The method of any one of claims 51 to 56, wherein the cells are engineered with an RNA-guided endonuclease.

58. The method of claim 57, wherein the RNA-guided endonuclease is selected from a Cas endonuclease, a Mad endonuclease, and a Cpf1 endonuclease.

59. The method of claim 58, wherein the RNA-guided endonuclease is Cas9 or Mad7.

60. The method of any one of any one of claims 51 to 59, wherein the method comprises disrupting a target gene and inserting the nucleotide sequence into the disrupted target gene, wherein disrupting the target gene comprises contacting the population of stem cells with (i) a gRNA targeting a target site in a target gene, and (ii) an RNA-guided endonuclease.

61. The method of claim 60, wherein the target gene is selected from B2M, TRAC and SIRPA.

62. The method of any one of any one of claims 51 to 61, comprising engineering the population of stem cells to be resistant to rapamycin.

63. The method of claim 62, wherein engineering the population of stem cells to be resistant to rapamycin comprises knocking out a FKBP12 gene.

64. The method of any one of claims 51, and 53-63, wherein the differentiation media comprises a BMP pathway activator, an FGF, a VEGF, and optionally a ROCK inhibitor.

65. The method of claim 64, wherein the BMP pathway activator is BMP4, the FGF is FGF2, the VEGF is VEGF-165, and the ROCK inhibitor is Y27632.

66. The method of any one of claims 64-65, wherein the differentiation media comprises SCF, TPO and LDL.

67. The method of any one of claims 51 and 53-63, wherein (d) comprises contacting the population of stem cell aggregates with the differentiation media for 1-5 days, wherein the differentiation media comprises a BMP pathway activator, an FGF, a VEGF, and optionally a ROCK inhibitor.

68. The method of any one of claims 51 and 53-63, wherein (d) comprises (i) contacting the stem cell aggregates for 1-5 days with the differentiation media comprising a BMP pathway activator, an FGF, a VEGF, and a ROCK inhibitor, to generate embryoid bodies or mesoderm cells, and (ii) contacting the embryoid bodies or mesoderm cells for 1-15 days with a differentiation media comprising the BMP pathway activator, the FGF, the VEGF, SCF, TPO, and the LDL.

69. The method of claim 68, wherein the differentiation media comprises 1-50 ng/mL BMP, 1-50 ng/mL FGF, 5-100 ng/mL VEGF, 0.1-20 uM ROCK inhibitor, 1-200 ng/mL SCF, 1-100 ng/mL TPO, and 1-50 ug/mL LDL, or any combination thereof.

70. The method of any one of claims 52-63, wherein the first differentiation media comprises a BMP pathway activator, an FGF, a VEGF, and optionally a ROCK inhibitor.

71. The method of any one of claims 52-63, wherein (d) comprises contacting the population of stem cell aggregates with the first differentiation media for 1-5 days, wherein the first differentiation media comprises a BMP pathway activator, an FGF, a VEGF, and optionally a ROCK inhibitor.

72. The method of any one of claims 52-63, wherein (d) comprises (i) contacting the stem cell aggregates for 1-5 days with a media comprising a BMP pathway activator, an FGF, a VEGF, and a ROCK inhibitor, to generate embryoid bodies or mesoderm cells, and (ii) contacting the embryoid bodies or mesoderm cells for 1-15 days with the first differentiation media comprising the BMP pathway activator, the FGF, the VEGF, SCF, TPO, and the LDL.

73. The method of any one of claims 67-72, wherein the BMP pathway activator is BMP4, the FGF is FGF2, the VEGF is VEGF-165, and the ROCK inhibitor is Y27632.

74. The method of any one of claims 52-73, wherein the second differentiation media comprises SCF, IL-7, IL-12, IL-15, FLT3L, a pyrimido-[4,5-b]-indole derivative, and an AhR inhibitor.

75. The method of any one of claims 52-73, wherein the second differentiation media comprises 1-100 ng/mL SCF, 1-50 ng/mL IL-7, 1-100 ng/mL IL-12, 1-100 ng/mL IL-15, 1-100 ng/mL FLT3L, 0.1-10 uM pyrimido-[4,5-b]-indole derivative, 0.1-10 uM AhR antagonist, and any combination thereof.

76. The method of claim 74 or 75, wherein the pyrimido-[4,5-b]-indole derivative is UM729 and the AhR inhibitor is SR1.

77. The method of any one of claims 52 to 67, wherein the first differentiation media and the second differentiation media are serum free.

78. The method of any one of claims 51 to 77, wherein the method is xenogenic-free.

79. The method of any one of claims 52 to 78, where the first differentiation media, and the second differentiation media each comprise the same base media.

80. The method of any one of claims 52 to 78, wherein the first differentiation media, and the second differentiation media each comprise different base media.

81. The method of any one of claims 52 to 78, wherein the first differentiation media and the second differentiation media each comprise a base media comprising Iscove's modified dulbecco's medium, bovine serum albumin, recombinant human insulin, human transferrin, and 2-mercaptoethanol.

82. The method of any one of claims 52 to 81, wherein the period of time of step (b) is 2-8 days, the period of time of step (c) is 1-5 days, the period of time of step (d) is 3-15 days, and the period of time of step (e) is 11-25 days.

83. The method of any one of claims 52 to 82, wherein steps (a)-(e) occur within 40-50 days.

84. The method of any one of claims 52 to 83, wherein the stem cells are induced pluripotent stem cells (iPSCs) or human embryonic stem cells (hESCs).

85. The method of any one of claims 51 to 84, wherein the population of hematopoietic progenitors comprises about 50% to about 100% CD34+/CD43+/CD45+ cells.

86. The method of any one of claims 52 to 85, wherein the population of NK cells comprises about 60% to about 100% CD43+/CD45+/CD56+/LFA1+ cells.

87. The method of any one of claims 52 to 78, comprising expanding the population of NK cells, wherein the population of NK cells expands about 1,000 to about 10,000 fold.

88. The method of any one of claims 51 to 87, wherein the population of stem cells is genetically engineered or edited.

89. The method of any one of claims 52 to 88, wherein the population of NK cells is genetically engineered or edited.

90. The method of any one of any one of claims 51 to 89, wherein the stem cells are iPSCs.

91. A population of cells produced by the method of any one of any one of claims 51 to 90.

92. A pharmaceutical composition comprising the cell population of claim 83.

93. A method of treating a cancer in a subject, comprising administering to the subject an effective amount of the population of cells of any one of claims 44-49 and 91, or the pharmaceutical composition of claim 50 or 84.

94. A kit comprising the population of cells of any one of claims 44-49 and 83 and instructions for administering the cell population to a subject in need thereof.

95. The kit of claim 94, wherein the subject has a cancer.


SEQUENCE LISTING (15 sequences)

SEQ ID NO: 1 - protein, 86 aa, Homo sapiens
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 2 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 195 aa, Homo sapiens
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 4 - protein, 285 aa, Homo sapiens
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 5 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 6 - protein, 93 aa, Homo sapiens [sequence omitted]

SEQ ID NO: 7 - protein, 93 aa, Homo sapiens
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 8 - protein, 21 aa, Homo sapiens
VVISVGSMGLIISLLCVYFWL

SEQ ID NO: 9 - protein, 19 aa, Homo sapiens
VAVAGCVFLLISVLLLSGL

SEQ ID NO: 10 - protein, 25 aa, Homo sapiens
PILLTISILSFFSVALLVILACVLW

SEQ ID NO: 11 - protein, 21 aa, Homo sapiens
GWNPHLLLLLLLVIVFIPAFW

SEQ ID NO: 12 - protein, 21 aa, Homo sapiens
MALPVTALLLPLALLLHAARP

SEQ ID NO: 13 - DNA, 1554 bp, synthetic construct
  source 1..1554: mol_type = other DNA
atggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - protein, 517 aa, synthetic construct
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 1560 bp, synthetic construct
  source 1..1560: mol_type = other DNA
gccaccatggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa